(19) United States
(12) Patent Application Publication
(10) Pub. No.: US 2009/0246170 A1
(43) Pub. Date: Oct. 1, 2009

(54) Therapeutic Agent For Alzheimer's Disease

(75) Inventors: Makoto Inoue, Ibaraki (JP); Yumiko Tokusumi, Ibaraki (JP); Hitoshi Iwasaki, Ibaraki (JP); Hiroto Hara, Ibaraki (JP); Toshiaki Tabata, Ibaraki (JP); Mamoru Hasegawa, Ibaraki (JP)

Correspondence Address: CLARK & ELBING LLP, 101 FEDERAL STREET, BOSTON, MA, 02110, US

(73) Assignee: DNAVEC Corporation, Tsukuba-shi, Ibaraki (JP)

(21) Appl. No.: 12/302,927

(22) PCT Filed: May 31, 2007
(86) PCT No.: PCT/JP2007/061058

§ 371 Date: Mar. 10, 2009

(30) Foreign Application Priority Data
May 31, 2006 (JP) 2006-151617
Apr. 19, 2007 (JP) 2007-110623

(51) Int. Cl. A61K 38/20; A61K 31/7088; A61K 38/19; A61K 38/02; A61P 25/28

(52) U.S. Cl. 424/85.2; 514/44 R; 424/85.1; 514/2

(57) ABSTRACT

The present invention provides novel therapeutic methods and agents for treating Alzheimer's disease. Specifically, the present invention relates to anti-inflammatory cytokines, anti-inflammatory cytokine genes, negative-strand RNA viral vectors carrying an anti-inflammatory cytokine gene, which are used for treating Alzheimer's disease or developing therapeutic agents for Alzheimer's disease. The present invention also provides pharmaceutical compositions for treating or preventing Alzheimer's disease, which comprise the cytokines or vectors. The present invention further provides methods for treating Alzheimer's disease, which comprise the step of administering an anti-inflammatory cytokine, or a vector such as a negative-strand RNA viral vector carrying an anti-inflammatory cytokine gene. The present invention enables novel gene therapies for Alzheimer's disease.

### TECHNICAL FIELD

The present invention relates to the treatment of Alzheimer's disease. Specifically, the present invention relates to the treatment of Alzheimer's disease using anti-inflammatory cytokines or vectors expressing anti-inflammatory cytokines, such as negative-strand RNA viral vectors carrying an anti-inflammatory cytokine gene.

### BACKGROUND ART

It has been reported that about 10% of the people aged 65 years or older suffer from senile dementia in Japan's rapidly aging society. Alzheimer's disease is one of the two major causes of dementia, and accounts for about 50% of the dementia. Alzheimer's disease is becoming a serious social problem including the problem of nursing care.

Agents currently used for the therapy of Alzheimer's disease include acetylcholine modulators such as activators of the acetylcholine system, and acetylcholine esterase inhibitors; β-amyloid modulators such as P-secretase (BACE) inhibitors, which inhibit generation of amyloid peptides, and inhibitors of amyloid peptide aggregation; neuroprotection and neurotrophic therapeutic agents such as neuropeptides and nerve growth factors; chelators; antioxidants; and anti-inflammatory agents.

In terms of therapeutic effect, therapeutic agents for Alzheimer's disease can be categorized into the following three types. First-generation drugs can hardly suppress the progression of dementia itself, although they can improve the intellectual function to some extent when used at earlier stages of Alzheimer's disease; second-generation drugs have the effect of improving intellectual function, and more than that, they are expected to have an effect on suppression of the progression of Alzheimer's disease; and third-generation drugs are radical preventive/therapeutic drugs.

Most of the drugs currently under evaluation are thought to be first-generation drugs. The “amyloid cascade hypothesis”, which ascribes senile plaque formation via aggregation and deposition of amyloid peptides as the cause of the disease, is widely accepted as the mechanism of onset and progression in Alzheimer's disease. Some of the drugs that target amyloid peptides are expected to be further developed into second- or third-generation drugs. Thus, currently, there are strong demands for second- and third-generation drugs as radical therapeutic drugs for Alzheimer's disease, as well as truly effective first-generation drugs.

On the other hand, the “inflammation hypothesis”, which indicates that enhanced activity of inflammatory microglia induces neuronal cell death in the brain with Alzheimer's disease, has been proposed as the mechanism of onset in Alzheimer's disease. In fact, the microglial activity is enhanced and microglia are accumulated particularly around senile plaques in the brains of patients with Alzheimer's disease. It has also been demonstrated that the lymphocytes infiltrate the brains of patients with Alzheimer's disease, and that substances which are activated upon inflammation, such as complements, are accumulated in the brains. From the analytical results of epidemiological investigation, it was expected that anti-inflammatory drugs, in particular, non-steroidal anti-inflammatory drugs (NSAIDs) suppress the progression of Alzheimer's disease. Furthermore, indomethacin was reported to significantly suppress the progression of Alzheimer's disease in pilot clinical trials (Rogers J et al., Neurology. 1993 August; 43(8):1609-11). Thus, large-scale clinical trials were conducted mainly for Cox-2-specific inhibitors which are less likely to cause gastrointestinal disorders. However, it has been reported in a one-year study of patients with mild to moderate Alzheimer's disease, that first-generation NSAIDs and new-generation NSAIDs could not be demonstrated to have effect of suppressing the progression in Alzheimer's disease (Aisen P S et al., JAMA. 2003 Jun. 4; 289(21):2819-26; Imbimbo BP. Expert Opin Investig Drugs. 2004 November; 13(11):1469-81; Townsend K P et al., FASEB J. 2005 October; 19(12):1592-601). It has also been reported that oral administration of a compound called MW01-5-188WH, which is a selective inhibitor of inflammation-induced cytokine production in glial cells, to mice suppresses the amyloid β1-42-induced up-regulation of interleukin-1β, tumor necrosis factor-α, and S100B in the hippocampus, recovers synaptic failures in the hippocampus, and improves hippocampus-dependent Y-maze behavior (Ralay Ranaivo H et al., J. Neurosci. 2006 Jan. 11; 26(2):662-70).

### [Non-Patent Document 1]

### Rogers J et al., Neurology. 1993 August; 43(8):1609-11

### [Non-Patent Document 2]

### Aisen P S et al., JAMA. 2003 Jun. 4; 289(21):2819-26

### [Non-Patent Document 3]

### Imbimbo B P. Expert Opin Investig Drugs. 2004 November; 13(11):1469-81

### [Non-Patent Document 4]

### Townsend K P et al., FASEB J. 2005 October; 19(12):1592-601

### [Non-Patent Document 5]

### Ralay Ranaivo H et al., J. Neurosci. 2006 Jan. 11; 26(2):662-70

### DISCLOSURE OF THE INVENTION

### Problems to be Solved by the Invention

The present invention was achieved in view of the circumstances described above. An objective of the present invention is to provide novel treatment methods and pharmaceutical agents for the therapy of Alzheimer's disease.

### Means for Solving the Problems

To achieve the above-described objective, the present inventors conducted dedicated studies to develop novel methods that are effective for treating Alzheimer's disease. Interleukin-10 (IL-10), which is one of the anti-inflammatory cytokines, regulates the inflammatory response by acting competitively against the activity of pro-inflammatory cytokines. It has been pointed out that in Alzheimer's disease, polymorphisms present in the promoter region of IL-10 are associated with the progression of the disease (Lio D et al., Genes Immun. 2003 April; 4(3):234-8; Scassellati C et al., Neurosci Lett. 2004 Feb. 12; 356(2):119-22; Arosio B et al., Neurobiol Aging. 2004 September; 25(8): 1009-15; Ma S L et al., Neurobiol Aging. 2005 July; 26(7):1005-10). However, there are no cases that examined such anti-inflammatory cytokines for the purpose of treating Alzheimer's disease. The present invention provides novel methods for treating Alzheimer's disease using anti-inflammatory cytokines or vectors expressing anti-inflammatory cytokine genes, such as negative-strand RNA viral vectors. The present invention provides novel gene therapy methods and such for treating and preventing Alzheimer's disease.

Specifically, the present invention relates to negative-strand RNA viral vectors carrying an anti-inflammatory cytokine gene for treating Alzheimer's disease or developing therapeutic agents for Alzheimer's disease; pharmaceutical compositions comprising the negative-strand RNA viral vectors for Alzheimer's disease; and methods for treating and preventing Alzheimer's disease using the negative-strand RNA viral vectors. More specifically, the present invention includes the following:

[1] a pharmaceutical composition for treating or preventing Alzheimer's disease, wherein the composition comprises

a negative-strand RNA viral vector carrying a gene encoding an anti-inflammatory cytokine or a partial peptide thereof, or

an anti-inflammatory cytokine or a partial peptide thereof;

[2] the composition of [1], wherein the composition comprises a negative-strand RNA viral vector carrying a gene encoding an anti-inflammatory cytokine or a partial peptide thereof;
[3] the composition of [1], wherein the composition comprises an anti-inflammatory cytokine or a partial peptide thereof;
[4] the composition of [1] or [2], wherein the negative-strand RNA viral vector is a paramyxovirus vector;
[5] the composition of [1] or [2], wherein the negative-strand RNA viral vector is a Sendai virus vector;
[6] the composition of any one of [1] to [5], wherein the anti-inflammatory cytokine is selected from the group consisting of interleukin-4, interleukin-10, interleukin-13, and partial peptides thereof;
[7] the composition of any one of [1] to [6], wherein the composition is used for nasal administration;
[8] a negative-strand RNA viral vector carrying a gene for an anti-inflammatory cytokine or a partial peptide thereof, wherein the vector is used for treating Alzheimer's disease or developing a therapeutic agent for Alzheimer's disease;
[9] an anti-inflammatory cytokine protein, wherein the protein is used for treating Alzheimer's disease or developing a therapeutic agent for Alzheimer's disease;
[10] the vector of [8], wherein the negative-strand RNA viral vector is a paramyxovirus vector;
[11] the vector of [8], wherein the negative-strand RNA viral vector is a Sendai virus vector;
[12] the vector of any one of [8], [10], and [11], wherein the anti-inflammatory cytokine is selected from the group consisting of interleukin-4, interleukin-10, interleukin-13, and partial peptides thereof;
[13] a method for treating or preventing Alzheimer's disease, wherein the method comprises the step of administering a negative-strand RNA viral vector carrying a gene encoding an anti-inflammatory cytokine or a partial peptide thereof, or an anti-inflammatory cytokine or a partial peptide thereof;
[14] the method of [13], wherein the administration is nasal administration.

The present invention also includes the following:

(1) a negative-strand RNA viral vector carrying a gene for an anti-inflammatory cytokine or a partial peptide thereof, wherein the vector is used for treating Alzheimer's disease or developing a therapeutic agent for Alzheimer's disease;
(2) the vector of (1), wherein the negative-strand RNA viral vector is a paramyxovirus vector;
(3) the vector of (1), wherein the negative-strand RNA viral vector is a Sendai virus vector;
(4) the vector of any one of (1) to (3), wherein the anti-inflammatory cytokine is selected from the group consisting of interleukin-4, interleukin-10, interleukin-13, and partial peptides thereof;
(5) a pharmaceutical composition for treating or preventing Alzheimer's disease, which comprises the vector of any one of (1) to (4); and
(6) the pharmaceutical composition of (5), which is used for nasal administration.

The present invention also relates to methods for treating or preventing Alzheimer's disease, which comprise the step of administering an anti-inflammatory cytokine or a vector encoding it. In particular, the present invention relates to methods for treating or preventing Alzheimer's disease, which comprise the step of administering a vector capable of expressing an anti-inflammatory cytokine, such as a negative-strand RNA viral vector carrying an anti-inflammatory cytokine gene. The negative-strand RNA viral vector is preferably a paramyxovirus vector, more preferably a Sendai virus vector. The anti-inflammatory cytokine is preferably IL-10. The administration is preferably nasal administration.

The present invention also relates to the use of an anti-inflammatory cytokine or a vector encoding it in the production of pharmaceutical agents for treating or preventing Alzheimer's disease. In particular, the present invention provides the use of an anti-inflammatory cytokine, and a vector carrying an anti-inflammatory cytokine gene, specifically, a negative-strand RNA viral vector carrying an anti-inflammatory cytokine gene in the production of pharmaceutical agents for treating or preventing Alzheimer's disease. The negative-strand RNA viral vector is preferably a paramyxovirus vector, more preferably a Sendai virus vector. The anti-inflammatory cytokine is preferably IL-10. The pharmaceutical agents comprising a negative-strand RNA viral vector are formulated into dosage forms suitable for nasal administration.

### EFFECTS OF THE INVENTION

The present invention provides novel therapeutic agents and methods for Alzheimer's disease using anti-inflammatory cytokines. In particular, the present invention provides therapeutic agents for Alzheimer's disease, which comprise negative-strand RNA viral vectors encoding an anti-inflammatory cytokine, and gene therapy methods for Alzheimer's disease using the vectors. The methods of the present invention can be novel therapeutic means that can be employed to substitute for or in combination with other therapeutic methods for Alzheimer's disease.

### BRIEF DESCRIPTION OF THE DRAWINGS

FIG. 1 depicts the result of measurement of the blood IL-10 level after administration of an SeV vector expressing IL-10. An SeV vector expressing LacZ was used as a control. Blood IL-10 was detected in a manner specific to the IL-10-expressing SeV vector and dependent on the dosage.

FIG. 2 depicts senile plaques in the parietal lobe of cerebral neocortex and the hippocampus (anti-Aβ antibody staining). The number of senile plaques (reddish brown spots) in the cerebral neocortex is evidently smaller in the SeV18+mIL10/TSΔF group (right) than that in the SeV18+LacZ/TSΔF group (left), both four weeks (upper panels) and eight weeks (lower panels) after the vector administration.

FIG. 3 depicts the ratio (%) of the senile plaque area to the entire cerebral neocortex area (mean±SE). The ratio (%) of the area of senile plaques to that of the entire cerebral neocortex eight weeks after the administration was determined using an image analysis software. The ratio was significantly lower in the SeV18+mIL10/TSΔF group than in the control group (the SeV18+LacZ/TSΔF group) (p<0.01, Student t test).

FIG. 4 depicts the activation of microglia in the olfactory bulb (Iba-1 staining). The number of activated microglia (reddish brown amoeboid cells) in the olfactory bulb is evidently increased in the SeV18+mIL10/TSΔF group (right) as compared to the SeV18+LacZ/TSΔF group (left), both four weeks (upper panels) and eight weeks (lower panels) after the administration.

FIG. 5 depicts the ratio (%) of the area of microglia to that of a single optical field in an olfactory bulb section stained with Iba-1 (mean±SE). The ratio (%) of the area of Iba-1-positive microglia to that of the single optical field in the olfactory bulb eight weeks after the administration was determined using an image analysis software. The ratio was significantly higher in the SeV18+mIL10/TSΔF group than in the control group (the SeV18+LacZ/TSΔF group) (p<0.01, Student t test).

FIG. 6 depicts the result of measurement of the Aβ level in the brain tissue after administration of an IL-10-expressing SeV vector. An SeV vector expressing LacZ was used as a control. In the group to which the SeV vector expressing IL-10 was administered, Aβ was shown to be decreased in most of the fractions. In particular, soluble Aβ40 (in TBS fraction and 1% Triton fraction) and insoluble Aβ42 (in formic acid fraction) were shown to be significantly decreased.

FIG. 7 depicts the result of measurement of the blood IL-10 levels in normal mice after administration of an SeV vector expressing IL-10. Blood IL-10 was detected in a vector dosage-dependent manner.

FIG. 8 depicts the kinetics of blood IL-10 level after administration of an SeV vector expressing IL-10. A single dose of nasal drop of SeV18+mIL10/TSΔF (5×10⁷ CIU/head) resulted in an AUC of 176,000 pg·h/ml.

FIG. 9 depicts IL-10 transfer into the brain after nasal administration of an SeV vector expressing IL-10. SeV18+mIL10/TSΔF or SeV18+LacZ/TSΔF was nasally administered to normal C57BL/6N mice (N=5) at 5×10⁸ CIU/head/53 μl. The same volume of DPBS(−) was administered as a control. Brain (divided into the following three parts: the olfactory bulb, cerebrum/hippocampus, and cerebellum/medulla oblongata), nasal mucosa, trachea/lung, and plasma were collected three days after the administration. mIL-10 in each tissue was quantified by ELISA. The expression levels of mIL-10 in olfactory bulb, cerebrum/hippocampus, and cerebellum/medulla oblongata shown in panel (A) are also shown in panel (B) which has a magnified scale of vertical axis.

FIG. 10 depicts IL-10 transfer into the brain after nasal administration of an SeV vector expressing IL-10. SeV18+mIL10/TSΔF or SeV18+LacZ/TSΔF was nasally administered to normal C57BL/6N mice (N=5) at 5×10⁸ CIU/head/53 μl. The same volume of DPBS(−) was administered as a control. Perfusion was performed, and the brains were collected three days after the administration. mIL-10 in the brain (divided into the following three parts: the olfactory bulb, cerebrum/hippocampus, and cerebellum/medulla oblongata), nasal mucosa, trachea/lung, and plasma was quantified by ELISA. The expression levels of mIL-10 in olfactory bulb, cerebrum/hippocampus, and cerebellum/medulla oblongata shown in panel (A) are also shown in panel (B) which has a magnified scale of vertical axis.

FIG. 11 depicts IL-10 transfer into the brain after nasal administration of an SeV vector expressing IL-10. SeV18+mIL10/TSΔF or SeV18+LacZ/TSΔF was nasally administered to normal C57BL/6N mice (N=5) at 5×10⁸ CIU/head/53 μl. The same volume of DPBS(−) was administered as a control. Brain (divided into the following three parts: the olfactory bulb, cerebrum/hippocampus, and cerebellum/medulla oblongata), nasal mucosa, trachea/lung, and plasma were collected after seven days from the administration. mIL-10 in each tissue was quantified by ELISA. The expression levels of mIL-10 in olfactory bulb, cerebrum/hippocampus, and cerebellum/medulla oblongata shown in panel (A) are also shown in panel (B) which has a magnified scale of vertical axis.

FIG. 12 depicts IL-10 transfer into the brain after nasal administration of an SeV vector expressing IL-10. SeV18+mIL10/TSΔF or SeV18+LacZ/TSΔF was nasally administered to normal C57BL/6N mice (N=5) at 5×10⁸ CIU/head/53 μl. The same volume of DPBS(−) was administered as a control. Perfusion was performed, and the brains were collected seven days after the administration. mIL-10 in the brain (divided into the following three parts: the olfactory bulb, cerebrum/hippocampus, and cerebellum/medulla oblongata), nasal mucosa, trachea/lung, and plasma was quantified by ELISA. The expression levels of mIL-10 in olfactory bulb, cerebrum/hippocampus, and cerebellum/medulla oblongata shown in panel (A) are also shown in panel (B) which has a magnified scale of vertical axis.

FIG. 13 depicts IL-10 transfer into the brain (concentration in CSF) after nasal administration of an SeV vector expressing IL-10. SeV18+mIL10/TSΔF (rats #1-#5) or SeV18+LacZ/TSΔF (rats #6-#10) was nasally administered to normal Wistar rats (N=5) at 1×10⁹ CIU/head/106 μl. The same volume of DPBS(−) was administered as a control (rats #11-#15). The plasma and cerebrospinal fluid were collected three days after the administration, and mIL-10 was quantified by ELISA.

FIG. 14 depicts the kinetics of blood IL-10 level in normal mice (C57BL/6N) after subcutaneous administration of the IL-10 protein. IL-10 from recombinant mice (2.0 μg/100 μl/head) was subcutaneously administered in the back, and blood mIL-10 was quantified by ELISA. The result is as follows: AUC=40,800 pg·h/ml; Cₘₐₓ=about 12,000 pg/ml; Tₘₐₓ=about 1.5 hr; and t_{1/2}=about 1 hr (initial value).

FIG. 15 depicts the IL-10 levels in APP mice after subcutaneous administration of the IL-10 protein.

FIG. 16 shows photographs depicting the effects of continuous subcutaneous administration of IL-10 to APP model mice (Tg2576). Results of anti-Aβ antibody (4G8) immunostaining of sections of the parietal lobe of cerebral neocortex and the hippocampus are shown. The upper panel shows the result for the IL-10 administration group, in which a small number of senile plaques (brown spots) were observed in the cerebral neocortex. The lower panel shows the result for the DPBS(−) administration group, in which many senile plaques were observed in the cerebral neocortex.

FIG. 17 depicts the result of semi-quantitative measurement of senile plaques in APP model mice (Tg2576) after continuous subcutaneous administration of IL-10. The scores for senile plaques were assigned according to the size as follows: large: nine points; middle: three points; small: one point. The total scores of senile plaques observed in the whole brain (excluding brain stem and cerebellum) were calculated. Statistical analysis between the groups was performed (Student's t test; mean±standard deviation).

FIG. 18 depicts the result of quantification of the area of senile plaques in the olfactory bulb, cerebral neocortex, and hippocampus of APP model mice (Tg2576) after continuous subcutaneous administration of IL-10 (Student t test). The “IL-10” and “DPBS(−)” bars indicate the results for the IL-10 and DPBS(−) administration groups, respectively.

### BEST MODE FOR CARRYING OUT THE INVENTION

The present invention relates to therapeutic agents and preventive agents for Alzheimer's disease, which comprise anti-inflammatory cytokines or vectors expressing anti-inflammatory cytokines. Such vectors include plasmids, naked DNAs, lyposome compositions, and viral vectors. In particular, the present invention relates to negative-strand RNA viral vectors carrying an anti-inflammatory cytokine gene for treating Alzheimer's disease or developing therapeutic agents for Alzheimer's disease, and pharmaceutical compositions that comprise the vectors for treating or preventing Alzheimer's disease. “Negative-strand RNA virus” (also referred to as “minus-strand RNA virus”) refers to a virus comprising a negative-strand (an antisense strand complementary to a sense strand encoding viral proteins) RNA as the genome. “Minus-strand RNA virus” is also referred to as “negative-strand RNA virus”. In particular, negative-strand RNA viruses that are preferably used in the present invention are negative single-stranded RNA viruses (also referred to as non-segmented negative-strand RNA viruses). “Negative single-stranded RNA virus” refers to a virus comprising a negative single-stranded RNA, i.e., a minus-strand RNA, as the genome. Such viruses include viruses belonging to Paramyxoviridae (including the genera Paramyxovirus, Morbillivirus, Rubulavirus, and Pneumovirus, etc.), Rhabdoviridae (including the genera Vesiculovirus, Lyssavirus, and Ephemerovirus, etc.), Filoviridae, and such. The negative-strand RNA viral vectors used in the present invention may be transmissible vectors or non-transmissible defective vectors. “Transmissible” means that, when a host cell is infected with a viral vector, the virus is replicated in the cell to produce infectious viral particles.

Specific examples of particularly preferred negative-strand RNA viruses suitable for use in the context of the present invention include, for example, Sendai virus, Newcastle disease virus, mumps virus, measles virus, respiratory syncytial virus (RS virus), rinderpest virus, distemper virus, simian parainfluenza virus (SV5), and human parainfluenza viruses 1, 2, and 3 belonging to Paramyxoviridae; influenza virus belonging to Orthomyxoviridae; and vesicular stomatitis virus and rabies virus belonging to Rhabdoviridae.

More preferably, paramyxoviruses are used in the present invention. “Paramyxoviruses” refers to viruses belonging to Paramyxoviridae, or derivatives of the viruses. Preferred paramyxoviruses include viruses belonging to Paramyxovirinae (including Respirovirus, Rubulavirus, and Morbillivirus), more preferably those belonging to the genus Respirovirus (also referred to as the genus Paramyxovirus) or derivatives thereof. The derivatives include viruses that are genetically-modified or chemically-modified in a manner not to impair their gene-transferring ability. Examples of viruses of the genus Respirovirus applicable to this invention are human parainfluenza virus-1 (HPIV-1), human parainfluenza virus-3 (HPIV-3), bovine parainfluenza virus-3 (BPIV-3), Sendai virus (also referred to as murine parainfluenza virus-1), and simian parainfluenza virus-10 (SPIV-10). In the context of the present invention, a more preferred paramyxovirus is the Sendai virus. These viruses may be derived from natural strains, wild strains, mutant strains, laboratory-passaged strains, artificially constructed strains, or the like.

Herein, “vector” refers to a carrier for introducing nucleic acids into cells. Negative-strand RNA viruses such as Sendai virus are excellent gene transfer vectors. In their life cycle, the vectors are transcribed and replicated only in the host cytoplasm. Since the vectors do not have any DNA phase, chromosomal integration does not occur. Therefore, safety issues such as oncogenic transformation and immortalization due to chromosomal aberration do not occur. This characteristic of negative-strand RNA viruses greatly contributes to safety when they are used as vectors. Results of foreign gene expression show that few nucleotide mutations are observed even after multiple continuous passages of Sendai virus. This indicates that the viral genome is highly stable and inserted foreign genes are stably expressed over a long period of time (Yu, D. et al., Genes Cells 2, 457-466 (1997)). Furthermore, the virus has qualitative advantages such as flexibility in the size and packaging of inserted genes due to the absence of a capsid structural protein.

The negative-strand RNA viral vector of the present invention may be, for example, a complex comprising the genomic RNA of a negative-strand RNA virus and viral proteins, namely, a ribonucleoprotein (RNP). Specifically, such an RNP is a complex comprising the genomic RNA of a negative-strand RNA virus, the N protein, P protein, and L protein. When RNPs are introduced into cells, cistrons encoding viral proteins are transcribed from the genomic RNA through the action of viral proteins, and the genome itself is replicated to form daughter RNPs. Thus, sustained expression of RNPs is expected. RNPs can be introduced into cells, for example, by combining the RNPs with a desirable transfection reagent. Replication of the genomic RNA can be confirmed by detecting the increase in the copy number of the RNA using RT-PCR, Northern blot hybridization, or such.

Alternatively, the negative-strand RNA viral vector of the present invention is preferably a negative-strand RNA viral particle. “Viral particle” refers to a microparticle comprising a nucleic acid, which is released from cells through the action of viral proteins. A negative-strand RNA viral particle has a structure in which the above-described RNP comprising the genomic RNA and viral proteins is enclosed in a lipid membrane (referred to as “envelope”) derived from the cell membrane. The viral particles may show infectivity. “Infectivity” refers to the ability of a negative-strand RNA viral vector, which has cell-adhesion and membrane-fusion abilities, to introduce a nucleic acid inside the vector into cells to which the vector has adhered. The negative-strand RNA viral vectors of the present invention may be transmissible vectors or defective non-transmissible vectors. “Transmissible” means that, when a host cell is infected with a viral vector, the virus is replicated in the cell to produce infectious viral particles.

The genomic RNA of a negative-strand RNA virus encodes a carried gene in the antisense direction. In general, the genome of a negative-strand RNA virus is constituted so that the viral genes are arranged in the antisense orientation between the 3′ leader region and the 5′ trailer region. “Transcription ending sequence (E sequence)-intervening sequence (I sequence)-transcription starting sequence (sequence)” exists between the ORFs of individual genes, which allows the RNAs encoding the ORFs of the genes to be transcribed as separate cistrons. The genomic RNA comprised in the vector of the present invention encodes the N (nucleocapsid (also referred to as nucleoprotein (NP)), P (phospho), and L (large) proteins in the antisense direction, which are viral proteins necessary for expression of the group of genes encoded by the RNA, and for autonomous replication of the RNA itself. The RNA may encode the M (matrix) protein, which is necessary for formation of viral particles. The RNA may also encode envelope proteins, which are necessary for infection of viral particles. The envelope proteins of negative-strand RNA virus include the F (fusion) protein, which causes cell membrane fusion, and the HN (hemagglutinin-neuraminidase) (or H (hemagglutinin)) protein, which is necessary for adhesion to cells. However, for certain cell types, the HN protein is not required for infection (Markwell, M. A. et al., Proc. Natl. Acad. Sci. USA 82(4):978-982 (1985)), and infection is achieved by just the F protein. The RNA may encode viral envelope proteins other than the F protein and/or HN protein.

For example, respective genes of each virus belonging to Paramyxovirinae are commonly represented as follows. In general, the NP gene is also represented as “N”. Furthermore, when “HN” has no neuraminidase activity, it is represented as “H (hemagglutinin)”.

The genus Respirovirus: NP P/C/V M F HN-L
The genus Rubulavirus: NP P/V M F HN(SH) L
The genus Morbillivirus: NP P/C/V M F H-L

The negative-strand RNA viral vectors of the present invention may lack any of the wild-type negative-strand RNA viral genes. The viral genomic RNA can replicate and express carried genes in cells as long as it encodes viral proteins (i.e., N, L, and P) necessary for RNP reconstitution, even when it does not encode any envelope-constituting protein. Such vectors include, for example, vectors that lack at least one of the genes encoding envelope-constituting proteins such as F, H, HN, G, M, and Ml, which vary depending on the type of virus (WO 00/70055 and WO00/70070; Li, H.-O. et al., J. Virol. 74(14) 6564-6569 (2000)). For example, a vector that lacks the M, F, or HN gene, or any combination thereof, can be preferably used as a paramyxovirus vector of the present invention. Such viral vectors can be reconstituted, for example, by externally supplying the missing gene products. The viral vectors prepared as described above adhere to host cells and cause cell fusion, as wild type viruses do. However, the viral vectors do not form daughter viral particles that retain the infectivity of the original vectors, since the vector genome introduced into the cells lacks some viral genes. Therefore, such vectors are useful as safe viral vectors for one-time gene introduction. Examples of genes deleted from the genome include the F gene and HN gene. In particular, vectors lacking at least the F gene are preferred in the present invention. For example, viral vectors can be reconstituted by transfecting host cells with a plasmid expressing a recombinant negative-strand RNA viral vector genome lacking the F gene, along with a vector expressing the F protein and a vector expressing the N, P, and L proteins (International Publication Numbers WO 00/70055 and WO 00/70070; Li, H O. et al., J. Virol. 74(14) 6564-6569 (2000)). Viruses can also be produced, for example, using host cells comprising F gene-integrated chromosomes. When these proteins are externally supplied, their amino acid sequences are not necessarily identical to those of virus-derived sequences. Mutations may be introduced into the proteins, and/or homologous genes from other viruses may be used as substitutes, as long as the activity of nucleic acid introduction is equivalent to or greater than that of naturally-occurring proteins.

Furthermore, amplification of the genomic RNA after introduction into cells can be prevented, when at least one of the genes encoding viral proteins (i.e., N, L, and P) necessary for RNP reconstitution is deleted or deficient. Such vectors can be produced by expressing the N, P, and L proteins in virus-producing cells.

The viral vectors of the present invention may be, for example, vectors that comprise, on the envelope surface thereof, proteins such as adhesion factors capable of adhering to specific cells, ligands, receptors and such, or antibodies or fragments thereof. Alternatively, the vectors may comprise chimeric proteins or such that have the above-mentioned proteins in their extracellular domain and polypeptides derived from the virus envelope in their intracellular domain. Vectors that target and infect specific tissues can thereby be produced. These proteins may be encoded by the viral genome, or may be supplied by expressing genes other than those in the viral genome (for example, genes carried by other expression vectors, or genes in the host chromosomes) at the time of viral vector reconstitution.

In the vectors of the present invention, any viral gene comprised may be altered from the wild type gene, for example, to reduce the immunogenicity of viral proteins, or to enhance the efficiency of RNA transcription or replication. Specifically, the transcriptional or replicational function of negative-strand RNA viral vector can be enhanced, for example, by altering at least one of the replication factor genes, N, P, and L. The HN protein, which is an envelope protein, has both hemagglutinin activity and neuraminidase activity. For example, the viral stability in blood can be enhanced by attenuating the hemagglutinin activity, and infectivity can be controlled by modifying the neuraminidase activity. The membrane fusion ability can be controlled by altering the F protein. Furthermore, for example, the antigen-presenting epitopes of the F protein or HN protein which may act as antigenic molecules on the cell surface can be analyzed, and this information can be used to prepare viral vectors that have a reduced antigenicity of these proteins. Furthermore, a temperature-sensitive mutation may be introduced into a viral gene to suppress release of secondarily released particles (or virus-like particles (VLPs)) (WO 2003/025570). For example, the following mutations can be introduced: G69E, T116A, and A183S for the M gene; A262T, G264, and K461G for the HN gene; L511F for the P gene; and N1197S and K1795E for the L gene. However, temperature-sensitive mutations that can be introduced are not limited thereto (see WO 2003/025570).

In the present invention, the genomic RNA of the above-described negative-strand RNA viral vector comprises a foreign gene encoding an anti-inflammatory cytokine. Herein, “anti-inflammatory cytokine” (also referred to as “anti-inflammation cytokine”) collectively refers to polypeptides that function to suppress inflammation, which include signaling molecules that promote signal transduction that leads to suppression of inflammation, and/or signaling molecules that inhibit signal transduction that leads to enhancement of inflammation (for example, pro-inflammatory cytokine inhibitors). Specifically, in the present invention, the anti-inflammatory cytokines include interleukin (IL)-4, IL-10, IL-11, IL-13, TGF-β, soluble TNF-α receptor, IL-1 receptor antagonist (IL-1ra), and other Th2 cytokines. “Th2 cytokines” refers to cytokines produced predominantly by type-2 helper T cells (Th2 cells) rather than by type-1 helper T cells (Th1 cells). Specifically, such Th2 cytokines include IL-4, IL-5, IL-6, IL-9, IL-10, and IL-13. An anti-inflammatory cytokine encoded by a vector may be a full-length natural polypeptide, or may be a partial peptide thereof (active fragment etc.), as long as it retains the activity. For example, deletion of N- or C-terminal amino acid residue(s) (for example, one to 30 amino acids, more specifically, one, two, three, four, five, ten, 15, 20, or 25 amino acids) probably has no influence on the cytokine activity. Alternatively, the anti-inflammatory cytokines may be polypeptides that inhibit signal transduction of pro-inflammatory cytokines, and include a soluble fragment of pro-inflammatory cytokine receptor (comprising a ligand-binding domain), or an antibody or antibody fragment that binds to the ligand-binding domain of a pro-inflammatory cytokine receptor. For the pro-inflammatory cytokine, a desired fragment comprising a mature polypeptide that lacks signal sequence can be used, and a desired protein signal sequence can be appropriately used as the N-terminal signal sequence for its secretion to the outside of cells. The secretory signal sequences include, for example, the signal sequences of desired secretory proteins such as interleukin (IL)-2 and tissue plasminogen activator (tPA), but are not limited thereto. Alternatively, the cytokine may be expressed as a protein fused to other peptide(s).

In the present invention, the anti-inflammatory cytokine is preferably selected from the group consisting of IL-4, IL-10, IL-13, and TGF-beta, and is more preferably IL-10. The nucleotide sequence of each cytokine gene and the corresponding amino acid sequence are known (IL-4: NM 000589, NP_{—}000580, AAH66277, AAH67515, NP_{—}758858, NP_{—}067258, and NP_{—}958427; IL-10: NM_{—}000572, NP_{—}000563, CAG46825, NP_{—}034678, and NP_{—}036986; IL-13: NM_{—}002188, NP_{—}002179, AAB01681, NP_{—}032381, and NP_{—}446280; and TGF-beta (transforming growth factor-beta): M_{—}60316).

Genes encoding anti-inflammatory cytokines can be obtained by hybridization using the above-exemplified anti-inflammatory cytokine genes, or such as probes. High-stringency conditions for hybridization include, for example, overnight prehybridization at 42° C. followed by overnight hybridization at 42° C. in a hybridization solution containing 25% formamide, 4×SSC, 50 mM Hepes (pH 7.0), 10×Denhardt's solution, and 20 μg/ml denatured salmon sperm DNA, or in a hybridization solution containing 50% formamide, 4×SSC, 50 mM Hepes (H 7.0), 10×Denhardt's solution, and 20 μg/ml denatured salmon sperm DNA for more stringent conditions. Post-hybridization wash may be carried out under the washing and temperature conditions of “1×SSC, 0.1% SDS, 37° C.” or such, “0.5×SSC, 0.1% SDS, 42° C.” or such for more stringent conditions, or “0.2×SSC, 0.1% SDS, 65° C.” for yet more stringent conditions. Furthermore, slight mutations causing no functional loss in protein can be introduced into natural cytokine genes by known methods. For example, site-directed mutations can be introduced by the PCR method, cassette mutagenesis method, or such. Alternatively, random mutations can be introduced by using chemical reagents, random nucleotides, or such. The amino acid sequence of an anti-inflammatory cytokine obtained by such method normally has a high homology to the amino acid sequence of the above-exemplified anti-inflammatory cytokine. “High homology” refers to sequence identity of at least 60% or more, preferably 80% or more, more preferably 90% or more, even more preferably at least 95% or more, and still more preferably at least 97% or more (for example, 98 to 99%). Such amino acid sequence identity can be determined, for example, using the BLAST algorithm by Karlin and Altschul (Proc. Natl. Acad. Sci. USA 87:2264-2268, 1990; and Proc. Natl. Acad. Sci. USA 90:5873-5877, 1993). When amino acid sequences are analyzed using BLASTX developed based on this algorithm (Altschul et al. J. Mol. Biol. 215: 403-410, 1990), the parameters are set, for example, as follows: score=50 and wordlength=3. When the BLAST and Gapped BLAST programs are used, the default parameters of each program are used. Specific procedures of these analytical methods are known (see the webpage of NCBI).

The activity of each anti-inflammatory cytokine can be detected by known methods. For example, methods for detecting activity by growth assay using the mouse mast cell MC/9 (ATCC CRL-8306), the human erythroleukemia cell line TF-1 (ATCC CRL-2003), or such are known (Thompson-Snipes, L. et al., 1991, J. Exp. Med. 173:507-510; Kruse N et al., EMBO J. 1993; 12:5121-5129; Oshima Y et al., J Biol Chem 2001; 276:15185-91; Oshima, Y., et al., J. Biol. Chem. 275, 14375-14380, 2000; Leland, P. et al., Oncol. Res. 7, 227-235, 1995). For example, various anti-inflammatory cytokine deletion mutants prepared using genetic recombination techniques can be assayed by the methods described above to identify active fragments. 50% effective dose (ED₅₀) is calculated. It is preferable to use partial peptides or such that have an activity of 50% or more, preferably 60% or more, 70% or more, 80% or more, 90% or more, or 95% or more when compared to the wild type.

There is no particular limitation on the origin of an anti-inflammatory cytokine encoded by the vector. The anti-inflammatory cytokine may be derived from any mammals, such as mice, rats, guinea pigs, rabbits, pigs, cattle, horses, donkeys, goats, dogs, chimpanzees, monkeys, and human. However, it is appropriate to use an anti-inflammatory cytokine derived from the same species as the subject of administration. As described above, the nucleotide sequences of the nucleic acids encoding such cytokines are available from databases. More specifically, for example, the sequence of mouse IL-10 is available under Genbank Accession Nos. AY410237 and NM_{—}010548, and the sequence of human IL-10 is available under Genbank Accession Nos. AY029171 and NM_{—}000572. For the site of inserting a foreign gene, a desired site can be selected, for example, within the non-protein-coding region of a virus genome. A foreign gene can be inserted, for example, between the 3′ leader region of genomic RNA and the viral protein ORF closest to the 3′ end, between the viral protein ORFs, and/or between the viral protein ORF closest to the 5′ end and the 5′ trailer region. Alternatively, in a genome in which the genes for envelope-constituting proteins, such as the M, F, or HN gene, are deleted, a foreign gene can be inserted into the deleted regions. When a foreign gene is introduced into a paramyxoviridae virus, the chain length of a polynucleotide fragment inserted into the genome is desirably a multiple of six (Kolakofski, D. et al., J. Virol. 1998: 72; 891-899; Calain, P. and Roux, L. J. Virol. 1993: 67; 4822-4830). An E-1-S sequence is arranged to be between the inserted foreign gene and the viral ORF. Two or more foreign genes can be inserted in tandem via E-1-S sequences.

In the present invention, “gene” refers to a genetic substance, i.e., a nucleic acid encoding a transcriptional unit. Nucleic acids include RNAs and DNAs. In the present invention, a nucleic acid encoding a polypeptide is referred to as a gene for the polypeptide. Furthermore, genes include those do not encode protein. For example, a gene may encode a functional RNA, such as a ribozyme or an antisense RNA. In general, a gene may be a naturally-occurring or artificially-designed sequence. In the present invention, “DNAs” includes both single-stranded and double-stranded DNAs. “Encoding a protein” means that a polynucleotide comprises an ORF that encodes an amino acid sequence of the protein in the sense or antisense direction, so that the protein can be expressed under appropriate conditions. In the present invention, “foreign gene” refers to a gene that is not carried by the wild-type virus from which a vector is derived.

Expression levels of a foreign gene carried by a vector can be controlled using the type of transcriptional initiation sequence added upstream (to the 3′-side of the minus strand) of the gene (WO01/18223). The expression levels can also be controlled by the position at which the foreign gene is inserted in the genome: the nearer the insertion position is to the 3′-end of the minus strand, the higher the expression level; conversely, the nearer the insertion position is to the 5′-end, the lower the expression level. Since it is generally advantageous to obtain high expression of an anti-inflammatory cytokine, it is preferable to link the anti-inflammatory cytokine-encoding gene to a highly efficient transcriptional initiation sequence, and to insert it near the 3′-end of the minus strand genome. Specifically, a foreign gene may be inserted between the 3′-leader region and the viral protein ORF closest to the 3′-end. Alternatively, a foreign gene may be inserted between the ORFs of the viral protein gene closest to the 3′-end and the second closest viral protein gene, or between the ORFs of the second and third closest viral protein genes. In wild type paramyxoviruses, the viral protein gene closest to the 3′-end of the genome is the N gene, the second closest gene is the P gene, and the third closest gene is the M gene. Alternatively, in those cases wherein a high level of expression of the antigen polypeptide is undesirable, the level of viral vector gene expression can be suppressed to obtain an appropriate effect, for example, by inserting the foreign gene at a site as close as possible to the 5′-side of the minus strand genome, or by selecting an inefficient transcriptional initiation sequence.

For example, a desired S sequence of a negative-strand RNA virus may be used as the S sequence to be attached when inserting a foreign gene-encoding nucleic acid into the genome.

The consensus sequence 3′-UCCCWVUUWC-5′ (W=A or C; V=A, C, or G) (SEQ ID NO: 1) can be preferably used for Sendai viruses. Particularly preferred sequences are 3′-UCCCAGUUUC-5′ (SEQ ID NO: 2), 3′-UCCCACUUAC-5′ (SEQ ID NO: 3), and 3′-UCCCACUUUC-5′ (SEQ ID NO: 4). When shown as plus strand-encoding DNA sequences, these sequences are 5′-AGGGTCAAAG-3′ (SEQ ID NO: 5), 5′-AGGGTGAATG-3′ (SEQ ID NO: 6), and 5′-AGGGTGAAAG-3′ (SEQ ID NO: 7). A preferred E sequence of a Sendai viral vector is, for example, 3′-AUUCUUUUU-5′ (SEQ ID NO: 8) or 5′-TAAGAAAAA-3′ (SEQ ID NO: 9) for the plus strand-encoding DNA. An I sequence may be, for example, any three nucleotides, specifically 3′-GAA-5′ (5′-CTT-3′ in the plus strand DNA).

As described above, the vector of the present invention may comprise another foreign gene at a position other than the position into which a gene encoding an anti-inflammatory cytokine is inserted. There is no limitation on such foreign genes. The foreign genes may be, for example, marker genes for monitoring vector infection, genes for cytokines, hormones, receptors, or antibodies that regulate the immune system, or fragments thereof, or other genes. The vectors of the present invention enable expression of anti-inflammatory cytokines via direct (in vivo) administration to a living body, or via indirect (ex vivo) administration which introduces a vector of the present invention into patient-derived cells or other cells and administers the cells to patients.

The negative-strand RNA viral vectors of the present invention do not encode the Aβ antigen. In other words, the negative-strand RNA viral vectors of the present invention do not comprise any nucleic acid encoding the Aβ antigen. The vectors of the present invention do not encode the Aβ antigen, and can therefore exert therapeutic effects on Alzheimer's disease. “Aβ antigen” refers to Aβ or an antigenic partial peptide thereof, and includes Aβ1-38, Aβ1-39, Aβ1-40, Aβ1-42, Aβ1-43, and Aβ1-44, and polypeptides comprising an antigenic partial fragment thereof. The negative-strand RNA viral vectors of the present invention do not encode, for example, polypeptides that comprise ten or more consecutive amino acids (preferably, nine, eight, seven, six, or five or more amino acids) from Aβ1-43 (DAEFRHDSGYEVHHQKLVFFAEDVGSNKGAIIGLMVGGVVIAT, SEQ ID NO: 10).

Recombinant negative-strand RNA viral vectors may be reconstituted using known methods. For example, such vectors can be produced by the steps of (a) transcribing DNA which encodes the genomic RNA of a negative-strand RNA virus encoding an anti-inflammatory cytokine, or the complementary strand thereof (antigenomic RNA, plus-strand), in mammalian cells or avian cells in the presence of viral proteins constituting RNP comprising the genomic RNA of the negative-strand RNA virus, and (b) collecting the produced negative-strand RNA viruses or RNP comprising the genomic RNA. The “viral proteins constituting RNP” mentioned above refers to proteins that form RNP together with the viral genomic RNA and constitute a nucleocapsid. These are a group of proteins necessary for genome replication and gene expression, and are typically N (nucleocapsid (also referred to as nucleoprotein (NP))-, P (phospho)-, and L (large)-proteins. Although these notations vary depending on viral species, corresponding proteins are known to those skilled in the art (Anjeanette Robert et al., Virology 247:1-6 (1998)). For example, “N” may be denoted as “NP”.

When reconstituting viruses, a negative-strand RNA genome (i.e. antisense strand, which is the same as the viral genome) or the plus-strand RNA (antigenome, the complementary strand of the genomic RNA) may be generated as described above. However, in order to increase the efficiency of vector reconstitution, the plus-strand is preferably generated. The viral genomic RNA may be deficient in genes encoding envelope-constituting proteins, as long as it encodes viral proteins required for RNP reconstitution. For example, the genomic RNA does not need to encode viral proteins, such as F, HN, and M, as long as it encodes the N, P, and L proteins. Such defective viruses can amplify the genomic RNA in cells, but do not release infectious virions, and thus are useful as highly safe gene transfer vectors (WO00/70055, WO00/70070, and WO03/025570; Li, H.-O. et al., J. Virol. 74(14) 6564-6569 (2000)). To produce a viral vector, the above envelope-constituting proteins are expressed separately in virus-producing cells to complement particle formation. In order to express viral proteins and RNA genome in cells, a vector linked with DNA that encodes the proteins or genome downstream of an appropriate promoter is introduced into host cells. The promoter used include, for example, CMV promoters (Foecking, M. K. and Hofstetter, H. (1986) Gene 45: 101-105), retrovirus LTRs (Shinnik, T. M., Lerner, R. A. and Sutcliffe (1981) Nature, 293, 543-548), EF1 promoters, and CAG promoters (Niwa, H. et al. (1991) Gene. 108: 193-199, and Japanese Patent Application Kokai Publication No. (JP-A) H3-168087 (unexamined, published Japanese patent application)).

The terminals of genomic RNA preferably reflect the terminals of the 3′-leader sequence and 5′-trailer sequence as accurately as possible, as in the natural viral genome. For example, a self-cleaving ribozyme is added at the 5′-end of the transcript to allow the ribozyme to accurately cleave off the end of the negative-strand RNA viral genome (Inoue, K. et al. J. Virol. Methods 107, 2003, 229-236). Alternatively, in order to accurately regulate the 5′-end of the transcript, the recognition sequence of bacteriophage RNA polymerase is used as a transcription initiation site, and the RNA polymerase is expressed within a cell to induce transcription. The bacteriophage RNA polymerase used include, for example, those of E. coli T3 phage and T7 phage, and Salmonella SP6 phage (Krieg, P. A. and Melton, D. A. 1987, Methods Enzymol. 155: 397-15; Milligan, J. F. et al., 1987, Nucleic Acids Res. 15: 8783-798; Pokrovskaya, I. D. and Gurevich, V. V., 1994, Anal. Biochem. 220: 420-23). Such bacteriophage RNA polymerases can be supplied using, for example, vaccinia viruses expressing the polymerases (Fuerst, T. R. et al., Proc. Natl. Acad. Sci. USA 83, 8122-8126 (1986), or supplied from expression vectors such as plasmids. To regulate the 3′-end of the transcript, for example, a self-cleaving ribozyme is encoded at the 3′-end of the transcript, allowing accurate cleavage of the 3′-end with this ribozyme (Hasan, M. K. et al., J. Gen. Virol. 1997:78:2813-2820; Kato, A. et al., EMBO J. 1997, 16: 578-587; and Yu, D. et al., Genes Cells 1997, 2: 457-466). An auto-cleaving ribozyme derived from the antigenomic strand of delta hepatitis virus can be used.

In the reconstitution of viruses in which the envelope-constituting protein genes have been deleted, the infectivity of produced viruses can be complemented by expressing the deleted envelope-constituting proteins and/or other envelope proteins in virus-producing cells. For example, the viruses may also be pseudotyped with envelope proteins of negative-strand RNA viruses of a different origin from the virus from which the viral vector genome is derived. Such an envelope protein used may be, for example, the G protein of vesicular stomatitis virus (VSV) (VSV-G) (J. Virology 39: 519-528 (1981)) (Hirata, T. et al., 2002, J. Virol. Methods, 104:125-133; Inoue, M. et al., 2003, J. Virol. 77:6419-6429; Inoue M. et al., J Gene Med. 2004; 6:1069-1081). Genes to be deleted from the genome include, for example, genes of spike proteins such as F, HN, H, and G, genes of envelope-lining proteins such as M, and any combinations thereof. Deletion of a spike protein gene is effective in rendering negative-strand RNA viral vectors nontransmissible, whereas deletion of the gene of an envelope-lining protein such as M protein is effective in disabling the particle formation from infected cells. For example, F gene-defective negative-strand RNA viral vectors (Li, H.-O. et al., J. Virol. 74, 6564-6569 (2000)), M gene-defective negative-strand RNA viral vectors (Inoue, M. et al., J. Virol. 77, 6419-6429 (2003)), and the like are preferably used. Moreover, greater safety would be assured with vectors defective in any combination of at least two of F, HN (or H) and M genes. For example, vectors lacking both M and F genes are nontransmissible and defective in particle formation while retaining high level infectivity and gene expression ability.

For instance, in an example of the production of F gene-defective recombinant viruses, a plasmid expressing a negative-strand RNA viral genome defective in F gene or a complementary strand thereof is transfected into host cells along with an expression vector expressing F protein and expression vectors for N, P, and L proteins. Alternatively, viruses can be more efficiently produced by using host cells in which the F gene has been incorporated into their chromosomes (WO00/70070). In this case, a sequence-specific recombinase such as Cre/loxP and FLP/FRT and a target sequence thereof are preferably used so that the F gene can be inducibly expressed (see WO00/70055, WO00/70070; Hasan, M. K. et al., 1997, J. General Virology 78: 2813-2820). Specifically, for example, the envelope protein genes are integrated into a vector having a recombinase target sequence, such as the Cre/loxP inducible expression plasmid pCALNdlw (Arai, T. et al., J. Virology 72, 1998, p 1115-1121). The expression is induced by, for example, infection with the adenovirus AxCANCre at an MOI of 3 to 5 (Saito et al., Nucl. Acids Res. 23: 3816-3821 (1995); and Arai, T. et al., J. Virol 72, 1115-1121 (1998)).

The negative-strand RNA viruses used in the present invention may be deficient in accessory genes. For example, by knocking out the V gene, one of the accessory genes of Sendai virus (SeV), the pathogenicity of SeV toward hosts such as mice is remarkably reduced without hindering gene expression and replication in cultured cells (Kato, A. et al., 1997, J. Virol. 71:7266-7272; Kato, A. et al., 1997, EMBO J. 16:578-587; Curran, J. et al.; WO01/04272; and EP1067179).

In addition, negative-strand RNA viruses used may include mutations in the P gene or L gene so as to enhance the persistence of infection. Specific examples of such mutations include mutation of Glu at position 86 (E86) of the SeV P protein, substitution of Leu at position 511 (L511) of the SeV P protein to another amino acid, or substitution of homologous sites in the P protein of a different negative-strand RNA virus. Specific examples include substitution of the amino acid at position 86 to Lys, and substitution of the amino acid at position 511 to Phe. Regarding the L protein, examples include substitution of Asn at position 1197 (N1197) and/or Lys at position 1795 (K1795) in the SeV L protein to other amino acids, or substitution of homologous sites in the L protein of another negative-strand RNA virus, and specific examples include substitution of the amino acid at position 1197 to Ser, and substitution of the amino acid at 1795 to Glu. Mutations of the P gene and L gene can significantly increase the effects of persistent infectivity, suppression of the release of secondary virions, and suppression of cytotoxicity.

Regarding more specific methods for the reconstitution of recombinant viruses, one can refer to, for example, the following references: WO97/16539; WO97/16538; WO00/70055; WO00/70070; WO01/18223; WO03/025570; Durbin, A. P. et al., 1997, Virology 235: 323-332; Whelan, S. P. et al., 1995, Proc. Natl. Acad. Sci. USA 92: 8388-8392; Schnell. M. J. et al., 1994, EMBO J. 13: 4195-4203; Radecke, F. et al., 1995, EMBO J. 14: 5773-5784; Lawson, N. D. et al., Proc. Natl. Acad. Sci. USA 92: 4477-4481; Garcin, D. et al., 1995, EMBO J. 14: 6087-6094; Kato, A. et al., 1996, Genes Cells 1: 569-579; Baron, M. D. and Barrett, T., 1997, J. Virol. 71: 1265-1271; Bridgen, A. and Elliott, R. M., 1996, Proc. Natl. Acad. Sci. USA 93: 15400-15404; Hasan, M. K. et al., J. Gen. Virol. 78: 2813-2820, 1997; Kato, A. et al., 1997, EMBO J. 16: 578-587; Yu, D. et al., 1997, Genes Cells 2: 457-466; Tokusumi, T. et al. Virus Res. 2002: 86; 33-38; Li, H.-O. et al., J. Virol. 2000: 74; 6564-6569. Following these methods, negative-strand RNA viruses including parainfluenza virus, vesicular stomatitis virus, rabies virus, measles virus, rinderpest virus, Sendai virus, and the like can be reconstituted from DNA.

The present invention provides methods for producing therapeutic and/or preventive agents for Alzheimer's disease (pharmaceutical compositions for treating and/or preventing Alzheimer's disease), which comprise the steps of:

(a) allowing transcription of a DNA that encodes the genomic RNA of a negative-strand RNA virus encoding an anti-inflammatory cytokine, or the complementary strand thereof (antigenomic RNA), in the presence of viral proteins that constitute an RNP comprising the genomic RNA of a negative-strand RNA virus in mammalian cells; and
(b) collecting the generated negative-strand RNA virus or RNP that comprises the genomic RNA.

The present invention also relates to the use of DNAs that encode the genomic RNA of a negative-strand RNA virus encoding an anti-inflammatory cytokine, or the complementary strand thereof (antigenome RNA), in producing therapeutic and/or preventive agents for Alzheimer's disease (pharmaceutical compositions for treating and/or preventing Alzheimer's disease). Furthermore, the present invention relates to therapeutic and/or preventive agents for Alzheimer's disease (pharmaceutical compositions for treating and/or preventing Alzheimer's disease), which comprise as an active ingredient, a negative-strand RNA virus encoding an anti-inflammatory cytokine. The present invention also relates to the use of anti-inflammatory cytokines, cells producing anti-inflammatory cytokines, nucleic acids encoding an anti-inflammatory cytokine, and cells into which a nucleic acid encoding an anti-inflammatory cytokine has been exogenously introduced, in producing therapeutic and/or preventive agents for Alzheimer's disease (pharmaceutical compositions for treating and/or preventing Alzheimer's disease). In addition, the present invention relates to therapeutic and/or preventive agents for Alzheimer's disease (pharmaceutical compositions for treating and/or preventing Alzheimer's disease), which comprise as an active ingredient, an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced.

Desired mammalian cells and the like can be used for virus production. Specific examples of such cells include cultured cells, such as LLC-MK2 cells (ATCC CCL-7) and CV-1 cells (for example, ATCC CCL-70) derived from monkey kidney, BHK cells (for example, ATCC CCL-10) derived from hamster kidney, and cells derived from humans. In addition, to obtain a large quantity of a virus vector, a viral vector obtained from an above-described host can be used to infect embryonated hen eggs to amplify the vector. Methods for manufacturing viral vectors using hen eggs have already been developed (Nakanishi, et al., ed. (1993), “State-of-the-Art Technology Protocol in Neuroscience Research III, Molecular Neuron Physiology”, Koseisha, Osaka, pp. 153-172). For example, a fertilized egg is placed in an incubator, and cultured for nine to twelve days at 37 to 38° C. to grow an embryo. After the viral vector is inoculated into the allantoic cavity, the egg is then cultured for several days (for example, three days) to proliferate the viral vector. Conditions, such as the period of culture, may vary depending upon the recombinant Sendai virus being used. Then, allantoic fluids, including the vector, are recovered. Separation and purification of a Sendai virus vector from allantoic fluids can be performed according to conventional methods (Tashiro, M., “Virus Experiment Protocol,” Nagai, Ishihama, ed., Medical View Co., Ltd., pp. 68-73, (1995)).

Titers of viruses recovered can be determined, for example, by measuring CIU (Cell Infectious Unit) or hemagglutination activity (HA) (WO 00/70070; Kato, A. et al., 1996, Genes Cells 1: 569-579; Yonemitsu, Y. and Kaneda, Y., Hemaggulutinating virus of Japan-liposome-mediated gene delivery to vascular cells. Ed. by Baker A H. Molecular Biology of Vascular Diseases. Method in Molecular Medicine: Humana Press: pp. 295-306, 1999). Titers of vectors carrying a marker gene such as GFP (green fluorescent protein) can be quantified (for example, as GFP-CIU) by directly counting infected cells, using the marker as an index. Titers thus determined can be treated in the same way as CIU (WO 00/70070).

The viral vectors can be purified to be substantially pure. Purification can be achieved using known purification/separation methods, including filtration, centrifugation, adsorption, and column purification, or any combinations thereof. The phrase “substantially pure” means that the virus component constitutes a major proportion of a solution of the viral vector. For example, a viral vector composition can be deemed “substantially pure” based on the fact that the proportion of protein contained as the viral vector component as compared to the total protein (excluding proteins added as carriers and stabilizers) in the solution is 10% (w/w) or greater, preferably 20% or greater, more preferably 50% or greater, preferably 70% or greater, more preferably 80% or greater, and even more preferably 90% or greater. Specific purification methods for the viral vectors include, for example, methods using cellulose sulfate ester or cross-linked polysaccharide sulfate ester (Japanese Patent Application Kokoku Publication No. (JP-B) S62-30752 (examined, approved Japanese patent application published for opposition), JP-B S62-33879, and JP-B S62-30753) and methods including adsorption to fucose sulfate-containing polysaccharide and/or degradation products thereof (WO97/32010). However, the invention is not limited thereto.

The present invention also relates to compositions for treating and preventing Alzheimer's disease, and developing therapeutic and preventive agents for Alzheimer's disease, which comprise an anti-inflammatory cytokine, cells producing an anti-inflammatory cytokine, a nucleic acid encoding an anti-inflammatory cytokine, and cells into which a nucleic acid encoding an anti-inflammatory cytokine has been exogenously introduced. In particular, the present invention relates to compositions for treating and preventing Alzheimer's disease, and developing therapeutic and preventive agents for Alzheimer's disease, which comprise a negative-strand RNA viral vector carrying an anti-inflammatory cytokine gene, cells producing the viral vector, or cells into which the vector has been introduced. When producing compositions comprising a vector, the vector may be combined with a desired pharmaceutically acceptable carrier or vehicle as needed. “Pharmaceutically acceptable carrier or vehicle” includes desired solutions in which the vector or cells can be suspended, for example, phosphate-buffered saline (PBS), sodium chloride solutions, Ringer's solution, and culture media. In the case where the vector is amplified using hen eggs, or such, it may contain allantoic fluid. Furthermore, compositions comprising the vector may comprise a carrier or vehicle such as deionized water and an aqueous solution of 5% dextrose. In addition, the compositions may also contain vegetable oils, suspending agents, surfactants, stabilizers, biocidal agents, or such. Preservatives or other additives may also be added. The compositions of the present invention do not comprise an Aβ antigen or a nucleic acid encoding an Aβ antigen. The vectors of the present invention and composition comprising them are useful in treating and preventing Alzheimer's disease, and developing therapeutic and preventive agents for Alzheimer's disease. The present invention relates to methods for producing therapeutic and preventive agents for Alzheimer's disease, which comprise the step of producing a composition comprising an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced, and a pharmaceutically acceptable carrier or vehicle. The present invention also relates to the use of an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced, in producing therapeutic and preventive agents for Alzheimer's disease. In addition, the present invention relates to methods for producing therapeutic and preventive agents for Alzheimer's disease, which comprise the step of producing a composition comprising a negative-strand RNA viral vector carrying an anti-inflammatory cytokine gene, cells producing the viral vector, or cells into which the vector has been introduced, and a pharmaceutically acceptable carrier or vehicle. The present invention also relates to the use of a negative-strand RNA viral vector carrying an anti-inflammatory cytokine gene, cells producing the viral vector, or cells into which the vector has been introduced, in producing therapeutic and preventive agents for Alzheimer's disease. By using vectors of the present invention, the blood level of an anti-inflammatory cytokine can be increased very efficiently, thereby achieving a high AUC (area under the pharmacokinetic curve). This enables effective suppression of Aβ accumulation and senile plaque formation.

Compositions comprising a vector of the present invention can be combined with, as a carrier, an organic substance such as a biopolymer, or an inorganic substance such as hydroxyapatite; specifically, a collagen matrix, a polylactate polymer or copolymer, a polyethylene glycol polymer or copolymer, and a chemical derivative thereof, etc.

Furthermore, compositions of the present invention, for example, compositions comprising a negative-strand RNA viral vector carrying an anti-inflammatory cytokine gene, may further comprise an anti-inflammatory cytokine or a nucleic acid encoding an anti-inflammatory cytokine. Such anti-inflammatory cytokines include those described herein and combinations thereof. Preferably, the anti-inflammatory cytokine is selected from the group consisting of IL-4, IL-10, and IL-13.

Moreover, the compositions of the present invention may comprise an adjuvant. For example, the use of a composition comprising a Th2 adjuvant can further promote a shift in the Th1/Th2 balance toward Th2. The term “Th2 adjuvant” refers to adjuvants which activate type II helper T cells (Th2 cells) more predominantly than type I helper T cells (Th1 cells). Specifically, aluminum hydroxide (alum), cholera toxin (B subunit), Schistosoma mansoni egg extract proteins (such as Lacto-N-fucopentaose III), and the like may be used (Grun, J. L. and P. H. Maurer, 1989, Cellular Immunology 121: 134-145; Holmgren J et al., 1993, Vaccine 11:1179-1184; Wilson A D et al., 1993, Vaccine 11:113-118; Lindsay D S et al, 1994, Int Arch Allergy Immunol 105:281-288; Xu-Amano J et al., 1993, J Exp Med 178:1309-1320; Okano M et al., 2001, J. Immunol. 167(1):442-50).

The negative-strand RNA viruses encoding an anti-inflammatory cytokine and the compositions of the present invention, such as compositions comprising the viruses, are used for treating or preventing Alzheimer's disease, or developing therapeutic or preventive agents for Alzheimer's disease. Herein, “used for treating or preventing Alzheimer's disease, or developing therapeutic or preventive agents for Alzheimer's disease” refers to exclusive use for treating or preventing Alzheimer's disease, or for development of therapeutic or preventive agents for Alzheimer's disease. “Development of therapeutic or preventive agents” means that at least one therapeutic or preventive effect on Alzheimer's disease is detected in a negative-strand RNA virus or a composition comprising the virus when its effectiveness either as a therapeutic or preventive agent is assessed. “Treatment of Alzheimer's disease” means amelioration of at least one symptom of Alzheimer's disease, including, for example, reduction of Aβ accumulation in brain tissues or blood, or decrease of senile plaques or their area. The compositions of the present invention are useful as agents for suppressing Aβ accumulation, in particular, agents for suppressing Aβ accumulation in brain tissues, blood, or such, as compared to when the compositions of the present invention are not administered. Alternatively, the compositions of the present invention are useful as agents for suppressing senile plaque, in particular, agents for reducing the number and/or the total area of senile plaques, as compared to when the compositions of the present invention are not administered. In the present invention, the treatment or prevention of Alzheimer's disease, or the development of a therapeutic or preventive agent for Alzheimer's disease does not comprise the step of administering an Aβ antigen or a nucleic acid encoding an Aβ antigen to an individual. The present invention relates to the use of an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced, in particular, a negative-strand RNA viral vector carrying an anti-inflammatory cytokine gene, for treating or preventing Alzheimer's disease, or for developing therapeutic or preventive agents for Alzheimer's disease. The present invention also relates to the use of an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced, in particular, a negative-strand RNA viral vector carrying an anti-inflammatory cytokine gene, in producing pharmaceutical compositions for treating or preventing Alzheimer's disease.

Furthermore, the present invention relates to packages and kits for preventing and/or treating Alzheimer's disease, which comprise vessels containing an anti-inflammatory cytokine, cells producing an anti-inflammatory cytokine, a nucleic acid encoding an anti-inflammatory cytokine, or cells into which a nucleic acid encoding an anti-inflammatory cytokine has been exogenously introduced. In particular, the present invention relates to packages and kits for preventing and/or treating Alzheimer's disease, which comprise vessels containing a negative-strand RNA viral vector carrying an anti-inflammatory cytokine gene. The vectors used may be those described herein. The vessels preferably have a configuration suitable to store active ingredients such as the negative-strand RNA viral vector carrying an anti-inflammatory cytokine gene in sterile conditions. Specifically, the vessels may be a glass or plastic ampule, vial, tube, bottle, syringe, or such. The packages and kits may further comprise an anti-inflammatory cytokine or another vector encoding an anti-inflammatory cytokine. The anti-inflammatory cytokine described herein and combinations thereof may be used. Preferably, the anti-inflammatory cytokine is selected from the group consisting of IL-4, IL-10, and IL-13. The packages and kits do not contain an Aβ antigen or a nucleic acid encoding an Aβ antigen. The negative-strand RNA viral vectors may be made into, for example, compositions suitable for nasal administration. The vessels, packages, and/or kits may contain descriptions or instructions on the use of active ingredients such as an anti-inflammatory cytokine, or a gene encoding the cytokine, for preventing and/or treating Alzheimer's disease. For example, kits comprising a negative-strand RNA viral vector carrying an anti-inflammatory cytokine gene may contain descriptions or instructions on the use of the vector for preventing and/or treating Alzheimer's disease. Furthermore, the vessels, packages, and/or kits may contain descriptions or instructions that neither an Aβ antigen nor a nucleic acid encoding an Aβ antigen is used in used in combination. The kits of the present invention are useful, for example, for suppression of Aβ accumulation, in particular, for suppressing Aβ accumulation in brain tissues, blood, or such, as compared to when the kits are not used. Furthermore, the kits of the present invention are useful for suppressing senile plaques, in particular, for reducing the number and/or the total area of senile plaques, as compared to when the kits are not used.

Alzheimer's disease can be treated and prevented by directly or indirectly administering an anti-inflammatory cytokine or a nucleic acid expressing an anti-inflammatory cytokine to an individual. In particular, Alzheimer's disease can be effectively treated and prevented by administering a negative-strand RNA virus carrying an anti-inflammatory cytokine gene or a composition comprising the virus to an individual. The present invention provides methods for treating and/or preventing Alzheimer's disease, which comprise the step of directly or indirectly administering an anti-inflammatory cytokine or a nucleic acid expressing an anti-inflammatory cytokine. In particular, the present invention provides methods for treating and/or preventing Alzheimer's disease, which comprise the step of administering a negative-strand RNA viral vector carrying an anti-inflammatory cytokine gene. The methods of the present invention do not comprise the step of administering an Aβ antigen or a nucleic acid encoding the antigen. The methods of the present invention enable the treatment of Alzheimer's disease without administering an exogenous Aβ antigen. The administration of an anti-inflammatory cytokine or a vector carrying an anti-inflammatory cytokine gene may be in vivo, or ex vivo via cells. When a negative-strand RNA viral vector is administered, the vector may be an infectious viral particle, a non-infectious viral particle, a viral core (an RNP complex containing a genome and genome-binding viral proteins), or such. In the present invention, “negative-strand RNA viral vector” refers to complexes that include a ribonucleoprotein (RNP) complex comprising the genomic RNA derived from the negative-strand RNA virus and viral proteins necessary for replicating the RNA and expressing the carried gene, and that replicate the genomic RNA and express the carried gene in infected cells. The RNP is, for example, a complex comprising the genomic RNA of a negative-strand RNA virus and the N, L, and P proteins. Thus, in the present invention, the negative-strand RNA viral vector includes viral infectious particles, noninfectious particles (virus-like particles; also referred to as VLP), and RNPs containing a genomic RNA and viral proteins binding to the genomic RNA, such as a nucleocapsid of the negative-strand RNA virus. RNP (viral core) that is a virion from which its envelope has been removed is, when introduced into cells, still capable of replicating the viral genomic RNA in the cells (WO97/16538; WO00/70055). RNP or VLP may be administered together with, for example, a transfection reagent (WO00/70055; WO00/70070).

To administer the negative-strand RNA viral vector via cells, the negative-strand RNA viral vector is introduced into appropriate cultured cells, cells collected from an inoculation subject animal, or the like. For infecting cells with the negative-strand RNA viruses outside the body (for example, in a test tube or dish), the infection is carried out in vitro (or ex vivo), in a desired physiological aqueous solution such as a culture solution or a physiological salt solution. Herein, MOI (multiplicity of infection; number of infectious viruses per cell) is preferably within a range of one to 1000, more preferably two to 500, yet more preferably three to 300, and even more preferably five to 100. The negative-strand RNA viruses and cells can be sufficiently contacted even for a short time. The contact may be carried out, for example, for one minute or more, and preferably three minutes or more, five minutes or more, ten minutes or more, or 20 minutes or more. The duration may be for example about one to 60 minutes, and more specifically about five to 30 minutes. Of course, the contact may be carried out for a longer duration than the above, such as several days or more.

Specific methods for introducing into cells RNPs or non-infectious viral particles (virus-like particles (VLPs)) that contain viral genomic RNAs, naked DNAs, plasmids, or such include those known to those skilled in the art, such as methods that utilize calcium phosphate (Chen, C. & Okayama, H. (1988) BioTechniques 6:632-638; Chen, C. and Okayama, H., 1987, Mol. Cell. Biol. 7: 2745), DEAE-dextran (Rosenthal, N. (1987) Methods Enzymol. 152:704-709), various liposome-based transfection reagents (Sambrook, J. et al. (1989) Molecular Cloning: A Laboratory Manual (Cold Spring Harbor Laboratory, Cold Spring Harbor, N.Y.)), or electroporation (Ausubel, F. et al. (1994) In Current Protocols in Molecular Biology (John Wiley and Sons, NY), Vol. 1, Ch. 5 and 9). Chloroquine may be added to the transfection to suppress the degradation in endosomes (Calos, M. P., 1983, Proc. Natl. Acad. Sci. USA 80: 3015). Transfection reagents include, for example, DOTMA (Roche), Superfect Transfection Reagent (QIAGEN, Cat No. 301305), DOTAP, DOPE, DOSPER (Roche #1811169), TransIT-LT1 (Mirus, Product No. MIR 2300), CalPhos™ Mammalian Transfection Kit (Clontech #K2051-1), and CLONfectin™ (Clontech #8020-1). Enveloped viruses are known to incorporate host cell-derived proteins during virion formation, and such proteins can potentially cause antigenicity and cytotoxicity when introduced into cells (J. Biol. Chem. (1997) 272, 16578-16584). It is thus advantageous to use RNPs without the envelope (WO 00/70055).

Moreover, virus RNPs can be directly produced in a cell by introducing into the cell an expression vector which expresses viral genomic RNAs and an expression vector which encodes viral proteins (the N, P, and L proteins) necessary for replicating the genomic RNAs. Cells into which a viral vector has been introduced may also be produced in such a manner.

Once cells into which a negative-strand RNA viral vector has been introduced are prepared, they are preferably cultured for about twelve hours to five days (preferably for one to three days) to express an anti-inflammatory cytokine from the vector. When a signal peptide has been added to the anti-inflammatory cytokine to be expressed from the vector, the cytokine can be secreted to the outside of the cells. The resulting cells may be administered to animals without further treatment or as a cell homogenate (lysate) containing the viral vector. To eliminate the growth potential, the cells may be treated with irradiation, ultraviolet radiation, a chemical agent, or such. A lysate of cells into which the vector has been introduced can be prepared by using methods of lysing the cell membrane with surfactants, methods in which freeze-thaw cycles are repeated, or such. The surfactants include non-ionic surfactants such as Triton X-100 and Nonidet P-40. The lysate may be administered in combination with transfection reagents.

The dosage of the composition of the present invention varies depending on the disease, patient's weight, age, sex, and symptom, the purpose of administration, the form of composition administered, the administration method, the gene to be introduced, and such. The dosage can be appropriately determined by those skilled in the art. The route of administration can be appropriately selected, which includes, for example, percutaneous, intranasal, transbronchial, intramuscular, intraperitoneal, intravenous, and subcutaneous administration, but is not limited thereto. In particular, a preferred administration includes intramuscular injection (for example, into gastrocnemius muscle), subcutaneous administration, intranasal administration (nasal drop), intracutaneous administration to the palm or sole, direct intrasplenic administration, intraperitoneal administration, and such. More preferred administration methods include intranasal administration, subcutaneous administration, and intramuscular administration. When an anti-inflammatory cytokine is administered, for example, subcutaneous administration is preferred. On the other hand, when a negative-strand RNA viral vector is administered, nasal administration (including administration using nasal drop, spray, catheter, etc.) is preferred. The number of inoculation sites may be one or more (for example, two to 15 sites). The dosage of inoculation may be appropriately adjusted depending on the subject animal to be inoculated, inoculation site, inoculation frequency, and such. The dosage of a cytokine protein may be about 8000 μg per kilogram body weight (8000 μg/kg) or less (Regul Toxicol Pharmacol. 2002 February; 35(1):56-71), preferably 25 μg/kg to 100 μg/kg (Pharmacol Rev. 2003 June; 55(2):241-69). When a negative-strand RNA viral vector is used, the vector is preferably administered at a dosage in the range of about 10⁵ CIU/ml to about 10¹¹ CIU/ml, more preferably about 10⁷ CIU/ml to about 10⁹ CIU/ml, still more preferably about 1×10⁸ CIU/ml to about 5×10⁸ CIU/ml, together with a pharmaceutically acceptable carrier. When converted into a virus titer, the single dose for human is 1×10⁴ CIU to 5×10¹¹ CIU (cell infectious unit), preferably 2×10⁵ CIU to 2×10¹⁰ CIU. The frequency of administration is once or more and within the range of clinically acceptable side effects. The same applies to the number of doses per day. Although one single administration can produce significant effects, the effects can be enhanced by performing administration twice or more. Furthermore, the anti-inflammatory cytokine itself may be administered additionally.

When the vector is inoculated via cells (ex vivo administration), for example, human cells, preferably autologous cells, can be infected with a negative-strand RNA viral vector, and 1×10⁴ to 10⁹ cells, preferably 1×10⁵ to 10⁸ cells, or a lysate of the cells can be administered. For non-human animals, for example, the dosage can be converted from the above-described dosage based on the body weight ratio or the volume ratio (e.g., mean value) of the target site for administration between the animal of interest and human, and administered. The subject to which a composition comprising a vector of the present invention is administered is preferably mammals (including human and non-human mammals). Specifically, the mammals include human, non-human primates such as monkeys, rodents such as mice and rats, rabbits, goats, sheep, pigs, cattle, dogs, and all other mammals. The subject animals for administration include animals and patients having at least one factor of Alzheimer's disease or at least one symptom of Alzheimer's disease. Such animals and patients include, for example, individuals with Alzheimer's disease, individuals with an increased amount of Aβ or enhanced Aβ deposition, individuals having an Alzheimer-type mutant gene, and Alzheimer's disease model animals.

The methods of the present invention ameliorate at least one symptom of Alzheimer's disease. Symptoms of Alzheimer's disease include, for example, enhancement of microglial activity; infiltration and/or accumulation of microglia in the brain, in particular, in senile plaques; accumulation of substances activated upon inflammation, e.g., complements, in the brain; accumulation and/or deposition of Aβ in the brain tissues; and impairment of learning and/or memory.

Furthermore, the present invention also relates to methods for assessing therapeutic effect on Alzheimer's disease, which comprise the steps of: administering a composition of the present invention, for example, a vector of the present invention or a composition comprising the vector, to an individual suffering from Alzheimer's disease; and detecting at least one symptom of Alzheimer's disease in the individual. The symptoms of Alzheimer's disease may be compared with a control that the composition of the present invention, for example, vector, is not administered. As described above, the symptoms of Alzheimer's disease to be assessed include enhancement of microglial activity; infiltration and/or accumulation of microglia in the brain, in particular, in senile plaques; accumulation of substances activated upon inflammation, e.g., complements, in the brain; accumulation and/or deposition of Aβ in the brain tissues; and impairment of learning and/or memory. The present invention also relates to methods for assessing therapeutic effect on Alzheimer's disease, which comprise the steps of: administering a composition of the present invention, for example, a vector of the present invention or a composition comprising the vector, to an individual; and detecting a symptom of Alzheimer's disease in the individual. The individual for administration includes those having at least one factor of Alzheimer's disease or at least one symptom of Alzheimer's disease, for example, individuals with Alzheimer's disease, individuals with an increased amount of Aβ or enhanced Aβ deposition, individuals having an Alzheimer-type mutant gene, and Alzheimer's disease model animals. When the composition is administered to individuals before the onset of Alzheimer's disease, control individuals to which the composition is not administered are monitored until they develop at least one symptom of Alzheimer's disease, and then the individuals to which the composition is administered are compared with the control individuals to evaluate the effects. Therapeutic and preventive effects on Alzheimer's disease can be monitored by using these methods.

### EXAMPLES

Hereinbelow, the present invention is specifically described with reference to the Examples; however, it should not be construed as being limited thereto. All the publications cited herein are incorporated as a part of the present specification.

### Example 1

### Construction of an F Gene-Deficient SeV Genomic cDNA Carrying the IL-10 Gene

### (1-1) Construction of a NotI Fragment of Each Gene (Addition of the Transcriptional Signal of Sendai Virus)

PCR was carried out using the two primers, pmIL10-N (SEQ ID NO: 13) and pmIL10-C (SEQ ID NO: 14), and cDNA of the mouse IL-10 (mIL-10) gene (Accession Number NM_{—}010548; SEQ ID NO: 11; the amino acid sequence is shown in SEQ ID NO: 12) as a template. The resulting product was digested with NotI, and was subcloned into pBluescript™ II KS (Stratagene), to construct an mIL-10 gene NotI fragment (SEQ ID NO: 15) to which the transcriptional signal of Sendai virus has been added.

SEQ ID NO: 11

ATGCCTGGCTCAGCACTGCTATGCTGCCTGCTCTTACTGACTGGCATGAG
GATCAGCAGGGGCCAGTACAGCCGGGAAGACAATAACTGCACCCACTTCC
CAGTCGGCCAGAGCCACATGCTCCTAGAGCTGCGGACTGCCTTCAGCCAG
GTGAAGACTTTCTTTCAAACAAAGGACCAGCTGGACAACATACTGCTAAC
CGACTCCTTAATGCAGGACTTTAAGGGTTACTTGGGTTGCCAAGCCTTAT
CGGAAATGATCCAGTTTTACCTGGTAGAAGTGATGCCCCAGGCAGAGAAG
CATGGCCCAGAAATCAAGGAGCATTTGAATTCCCTGGGTGAGAAGCTGAA
GACCCTCAGGATGCGGCTGAGGCGCTGTCATCGATTTCTCCCCTGTGAAA
ATAAGAGCAAGGCAGTGGAGCAGGTGAAGAGTGATTTTAATAAGCTCCAA
GACCAAGGTGTCTACAAGGCCATGAATGAATTTGACATCTTCATCAACTG
CATAGAAGCATACATGATGATCAAAATGAAAAGCTAA

SEQ ID NO: 13

ACTTGCGGCCGCCAAAGTTCAATGCCTGGCTCAGCACTGCTATGCTGCCT
G

SEQ ID NO: 14

ATCCGCGGCCGCGATGAACTTTCACCCTAAGTTTTTCTTACTACGGTTAG
CTTTTCATTTTGATCATCATGTATGCTTC

SEQ ID NO: 15

gcggccgccaaagttcaATGCCTGGCTCAGCACTGCTATGCTGCCTGCTC
TTACTGACTGGCATGAGGATCAGCAGGGGCCAGTACAGCCGGGAAGACAA
TAACTGCACCCACTTCCCAGTCGGCCAGAGCCACATGCTCCTAGAGCTGC
GGACTGCCTTCAGCCAGGTGAAGACTTTCTTTCAAACAAAGGACCAGCTG
GACAACATACTGCTAACCGACTCCTTAATGCAGGACTTTAAGGGTTACTT
GGGTTGCCAAGCCTTATCGGAAATGATCCAGTTTTACCTGGTAGAAGTGA
TGCCCCAGGCAGAGAAGCATGGCCCAGAAATCAAGGAGCATTTGAATTCC
CTGGGTGAGAAGCTGAAGACCCTCAGGATGCGGCTGAGGCGCTGTCATCG
ATTTCTCCCCTGTGAAAATAAGAGCAAGGCAGTGGAGCAGGTGAAGAGTG
ATTTTAATAAGCTCCAAGACCAAGGTGTCTACAAGGCCATGAATGAATTT
GACATCTTCATCAACTGCATAGAAGCATACATGATGATCAAAATGAAAAG
CTAAccgtagtaagaaaaacttagggtgaaagttcatcgcggccgc

(1-2) Construction of an F Gene-Deficient SeV cDNA Carrying the mIL-10 Gene

A cDNA (pSeV18+NotI/ΔF) of F gene-deficient SeV vector (WO 00/70070) was digested with NotI. The mIL-10 gene NotI fragment was inserted into the NotI site to construct an F gene-deficient SeV cDNA carrying the IL-10 gene (pSeV18+mIL10/ΔF).

### Example 2

### Reconstitution and Amplification of Sendai Virus Vector

Virus reconstitution and amplification were carried out according to the report of Li et al. (Li, H.-O. et al., J. Virology 74. 6564-6569 (2000), WO 00/70070) and a modified method thereof (WO 2005/071092). Helper cells for the F protein, in which expression of the F protein can be induced by the Cre/loxP expression induction system, were used for producing vectors. This system uses the pCALNdLw plasmid (Arai, T. et al., J. Virol. 72: 1115-1121 (1988)), which has been designed in a way that expression of gene products is induced by the Cre DNA recombinase. To express the inserted gene, a transformant with the plasmid was infected with a recombinant adenovirus expressing the Cre DNA recombinase (AxCANCre) by the method of Saito et al. (Saito, I. et al., Nucl. Acid. Res. 23, 3816-3821 (1995), Arai, T. et al., J. Virol. 72, 1115-1121 (1998)).

An F gene-deficient SeV vector (abbreviated as SeV18+mIL10/ΔF) carrying the mouse IL-10 gene (hereinafter abbreviated as mIL-10) was prepared by the method described above. The genomic RNA (minus strand) of SeV18+mIL10/ΔF is shown in SEQ ID NO: 16, and the antigenomic RNA (plus strand) is shown in SEQ ID NO: 17. Furthermore, the genomic RNA (minus strand) sequence of an F gene-deficient Sendai virus vector expressing IL-10 (hereinafter abbreviated as SeV18+mIL10/TSΔF), which has the G69E, T116A, and A183S temperature-sensitive mutations in the M protein, the A262T, G264, and K461G temperature-sensitive mutations in the HN protein, the L511F mutation in the P protein, and the N1197S and K1795E mutations in the L protein, is shown in SEQ ID NO: 18. The antigenomic RNA (plus strand) sequence thereof is shown in SEQ ID NO: 19. As controls, an F gene-deficient SeV vector carrying the E. coli LacZ gene (abbreviated as SeV18+lacZ/ΔF) and an F gene-deficient SeV vector of temperature-sensitive mutation type (hereinafter abbreviated as SeV18+LacZ/TSΔF) were produced by the same method as described above.

### Example 3

### Therapeutic Effects of Nasal Drop (Nasal) Administration of SeV-mIL10/ΔF in Alzheimer's Disease Model Animal

### (3-1) Animal and Administration Method

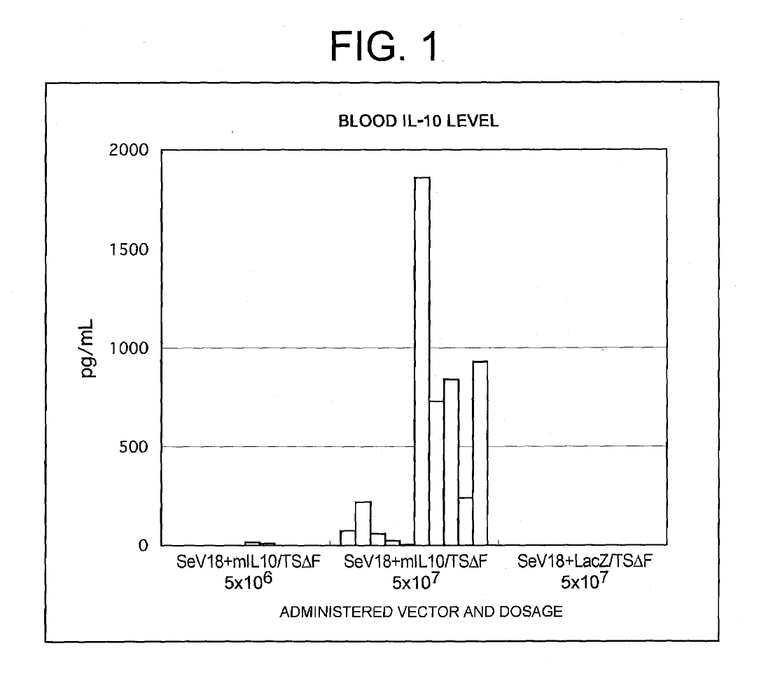

The therapeutic and preventive effects of SeV18+mIL10/ΔF of the present invention on Alzheimer's disease can be assessed by using Alzheimer's disease model mice (hereinafter referred to as APP mice) such as APP transgenic mice (Tg2576; Hsiao K et al., Science, 1996, 274: 99-102). Mice were divided into two groups, each containing four mice; one was the treatment group and the other was the control group. The body weights of the APP transgenic mice used in this assessment were about 20 g. 5×10⁶ CIU of SeV18+mIL10/ΔF or 5×10⁶ CIU of SeV18+lacZ/ΔF was intranasally (nasally) administered to each animal in the treatment group or the control group, respectively. An example of the experiment using SeV18+mIL10/TSΔF and SeV18+LacZ/TSΔF is as follows. 16-month-old APP mice (Tg2576) (female) were divided into three groups, each containing ten mice. For one animal in each group, 5×10⁶ CIU/201/head of SeV18+mIL10/TSΔF, 5×10⁷ CIU/20 μl/head of SeV18+mIL10/TSΔF, or 5×10⁷ CIU/20 μl/head of SeV18+LacZ/TSΔF was intranasally administered. Before the administration and three days after the administration, blood was collected from the mice, and plasma was prepared. The plasma IL-10 level was determined using the mouse IL10 ELISA Kit Quantikine (R&D Systems) according to the appended protocol. The mouse plasma was diluted 50-fold with the dilution buffer attached to the kit. The plasma IL-10 level before the administration was lower than or comparable to the detection limit (4 pg/ml). The plasma IL-10 level three days after the administration is shown in FIG. 1. Plasma IL-10 was detected in a dosage-dependent manner, although the level varies among animals.

### (1) Senile Plaque Elimination Effect

A Sendai virus vector was intranasally (nasally) administered to mice. The mice were dissected eight weeks after the administration. Brain tissue sections can be prepared from regions such as the cortex of frontal lobe, parietal lobe, and hippocampus. The experiment described below can be conducted using the cryosections. To detect the Aβ protein or senile plaques in the tissues, the sections were treated with 70% formic acid, and endogenous peroxidases were inactivated with 5% H₂O₂. After reaction with a rabbit anti-pan-Aβ antibody (1000-fold dilution), a peroxidase-labeled secondary antibody was added, and then DAB staining was performed. The area of Aβ accumulation in each region was measured, and then the ratio of the area of Aβ accumulation that occupies in each measured site was calculated.

Specifically, the effect of SeV18+mIL10/TSΔF was assessed using 12-month-old female APP transgenic mice (Tg2576) (Hsiao K et al., Science, 1996, 274: 99-102). Mice were divided into two groups, each containing 15 mice; one was the treatment group and the other was the control group. 5×10⁶ CIU of SeV18+mIL10/TSΔF or 5×10⁵ CIU of SeV18+LacZ/TSΔF was intranasally (nasally) administered to a animal in the treatment group or the control group, respectively, under light anesthesia with sevoflurane. Four weeks after the treatment, blood was collected from five mice from the SeV18+mIL10/TSΔF group and four mice from the SeV18+LacZ/TSΔF group, and then the mice were dissected. Eight weeks after the treatment, blood was collected from ten mice from the SeV18+mIL10/TSΔF group and nine mice from the SeV18+LacZ/TSΔF group, and then the mice were dissected. The brain with the olfactory bulb was vertically divided into the right and left halves. One was rapidly frozen and stored for biochemical measurement, and the other was immersed and fixed in 4% paraformaldehyde solution for histopathological examination. Paraffin-embedded histopathological sections were prepared as vertical sections containing the olfactory bulb at 1 mm from the midline. The sections were stained with hematoxylin and eosin for standard histopathological examination, and observed under a microscope. Immunostaining with an anti-Aβ antibody (4G8) was performed to detect the Aβ protein and senile plaques in the tissues. Alternatively, Iba-1 immunostaining was performed to stain microglia and macrophages.

Samples stained with the anti-Aβ antibody were divided into the following four parts: the olfactory bulb, cerebral neocortex, hippocampus, and brain stem/cerebellum. The quantity of senile plaques and blood vessels positive for anti-Aβ antibody staining that were present in each region were evaluated under a light microscope. The area of senile plaques in the cerebral neocortex was quantified by image analysis software (NIH Image, Japanese Edition) using recorded images with the same magnification.

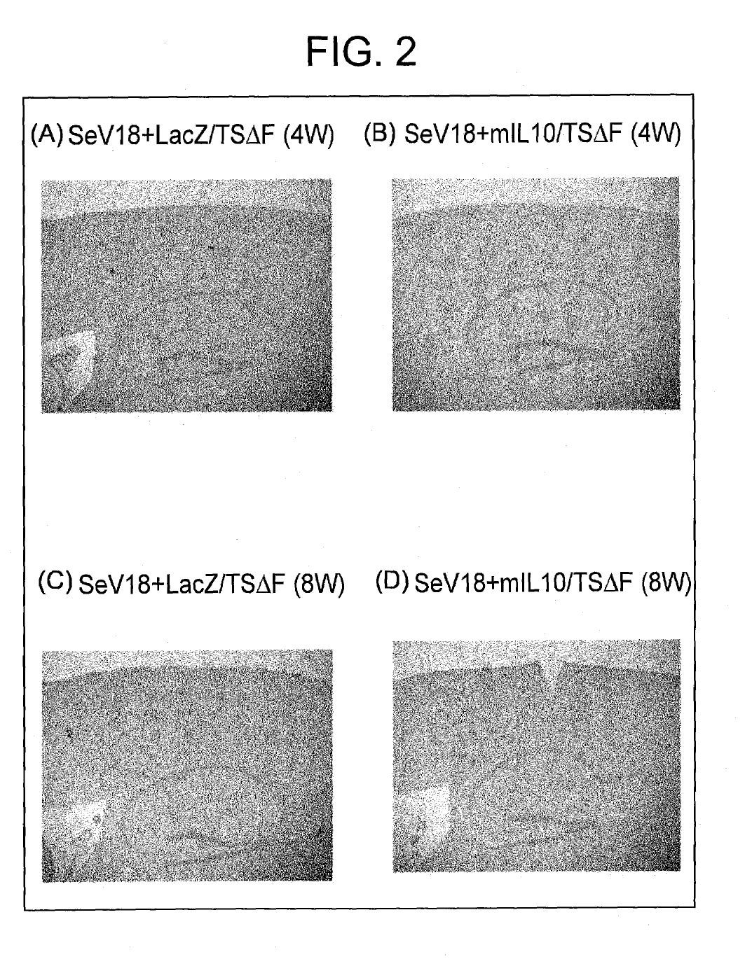

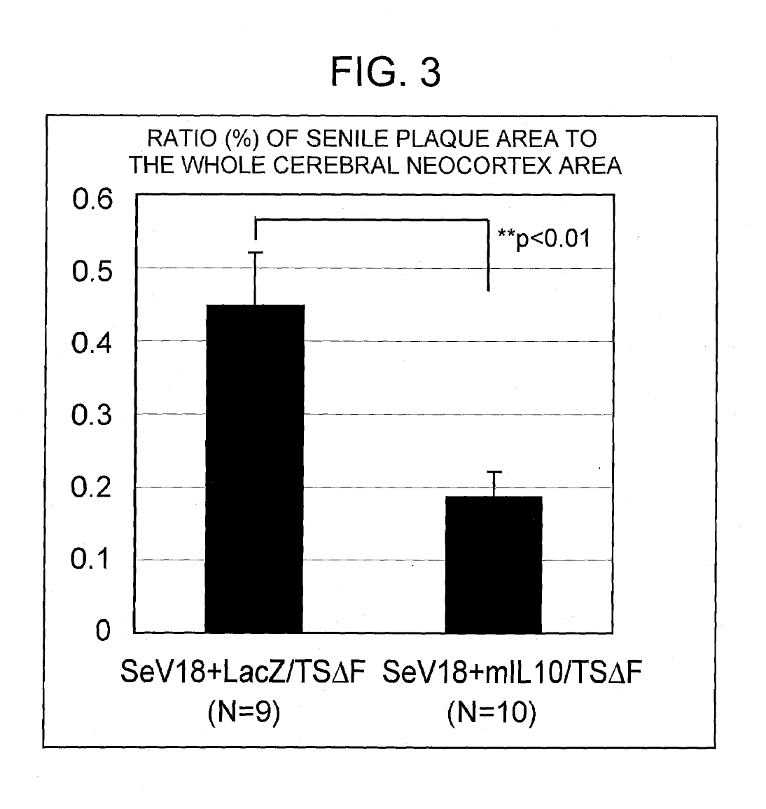

An example of the result of staining sections is shown in FIG. 2 (the parietal lobe of cerebral neocortex and hippocampus). Meanwhile, the area ratio of Aβ deposition is shown in FIG. 3. Since only a small number of senile plaques were formed in the hippocampus of both groups, there was no significant difference between the groups. In contrast, eight weeks after the treatment, the area of senile plaques in the cerebral neocortex was clearly reduced in the SeV18+mIL10/TSΔF group. The difference was statistically significant (p<0.01).

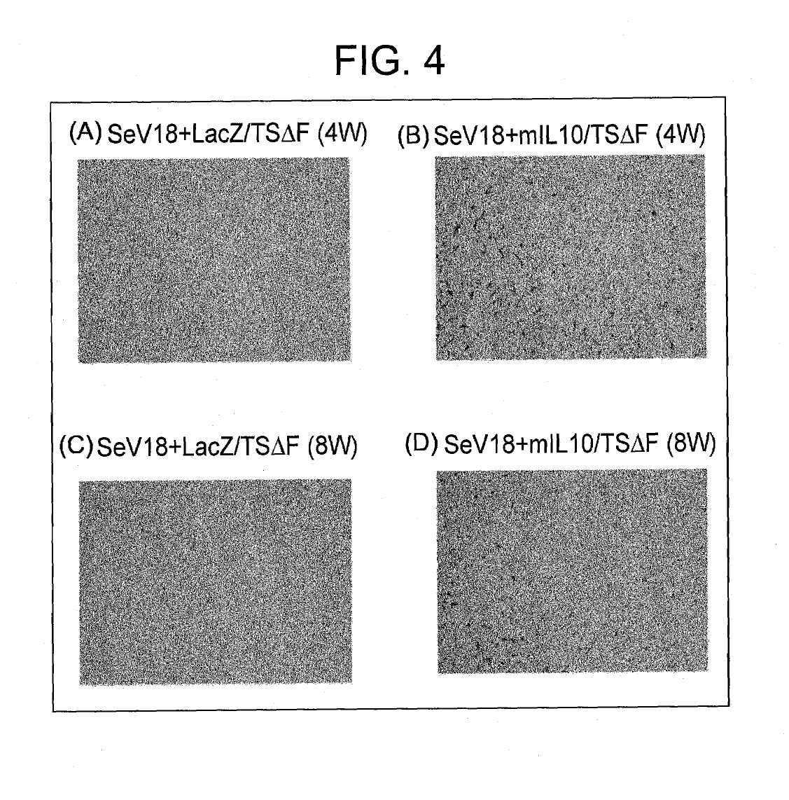

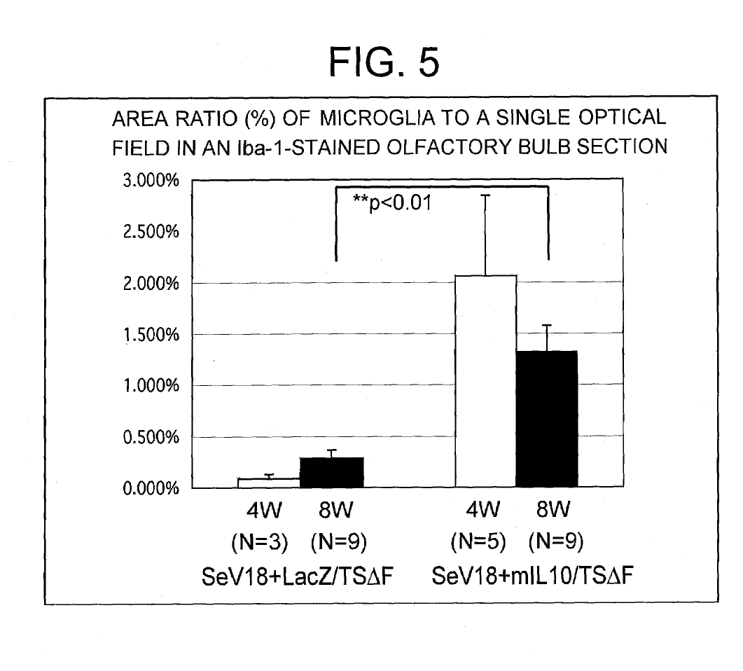

On the other hand, in the Iba-1 immunostaining samples, a clear difference in the number of activated microglia in the olfactory bulb was observed, although there was no clear difference observed in the cerebral neocortex, hippocampus, or brain stem/cerebellum. As shown in FIG. 4, the number of activated microglia was increased in the SeV18+mIL10/TSΔF group four and eight weeks after the treatment. According to the result of the analysis of recorded images with the same magnification, as shown in FIG. 5, the area ratio of Iba-1-positive cells was statistically significantly increased in the SeV18+mIL10/TSΔF group eight weeks after the treatment (p<0.0, Student t test). This suggests that, in the SeV18+mIL10/TSΔF group, activated microglia or macrophages actively eliminate foreign materials (the majority of them are dead olfactory cells infected with SeV) from the olfactory bulb. This also suggests that the mIL-10 protein expressed in nasal mucosa cells is transported along axons of olfactory cells in the opposite direction to the olfactory bulb, and the microglial activation is promoted in the olfactory bulb. Since the number of images analyzed for each group was small and different, statistical analysis was not performed. However, the number of microglia in the SeV18+mIL10/TSΔF group was clearly larger than that of the control group (SeV18+LacZ/TSΔF group) even just four weeks after the administration.

### (2) Assay of Aβ in Brain Tissues

Mouse cerebrum and cerebellum were cut along the fissura mediana. A hemisphere was rapidly frozen and stored at −80° C. The brain hemisphere was homogenized in 1 ml of TBS solution. The homogenate was centrifuged at 100,000 g in a bench-top ultracentrifuge for one hour. The soluble fraction (TBS fraction) was stored, while the insoluble fraction was dissolved in 2% SDS, homogenized, and then centrifuged at 100,000 g for one hour. The soluble fraction (2% SDS fraction) was stored, while the insoluble fraction was dissolved in 70% formic acid, homogenized, and then centrifuged at 100,000 g for one hour. The soluble fraction (formic acid fraction) was stored. An ELISA kit from Biosource was used to assay Aβ40 and 42 in brain tissues. The TBS fraction was diluted four-fold; the 2% SDS fraction was diluted 400- to 2000-fold; and the formic acid fraction was diluted 1000-fold in 1 M Tris solution. Then, the diluted fractions were further diluted (2- to 10-fold) with ELISA dilution buffer and assayed.

Specifically, 10-month-old APP mice (Tg2576) were divided into two groups, each containing ten mice. SeV18+mIL10/TSΔF or SeV18+LacZ/TSΔF was intranasally (nasally) administered to each group at 5×10⁶ CIU/20 μl/head per animal. Eight weeks after the administration, the brains were excised and the right hemispheres were used. Using Teflon® homogenizer, each mouse brain was homogenized in five volumes of TBS (50 mM Tris-HCl (pH 7.6), 150 mM NaCl) containing a protease inhibitor cocktail (CALBIOCHEM). The homogenate was centrifuged at 100,000 g for one hour at 4° C. The supernatant was collected and stored at −80° C. (TBS fraction). The precipitate was homogenized using a hand homogenizer in three volumes of 1% Triton X-100/TBS (containing protease inhibitors) to one volume of the mouse brain. The homogenate was incubated at 37° C. for 15 minutes, and then centrifuged at 100,000 g for one hour at 4° C. The supernatant was collected and stored at −80° C. (1% Triton fraction). Furthermore, the precipitate was homogenized using a hand homogenizer in three volumes of 2% SDS/TBS (containing protease inhibitors) to one volume of the mouse brain. The homogenate was incubated at 37° C. for 15 minutes, and then centrifuged at 100,000 g for one hour at 25° C. The supernatant was collected and stored at −80° C. (2% SDS fraction). Finally, using a hand homogenizer, the precipitate was homogenized in the same volume of 70% formic acid as the mouse brain. The homogenate was centrifuged at 100,000 g for one hour at 4° C. The supernatant was collected, and its pH was adjusted by adding ten volumes of 1 M Tris. The supernatant was then stored at −80° C. (FA fraction).

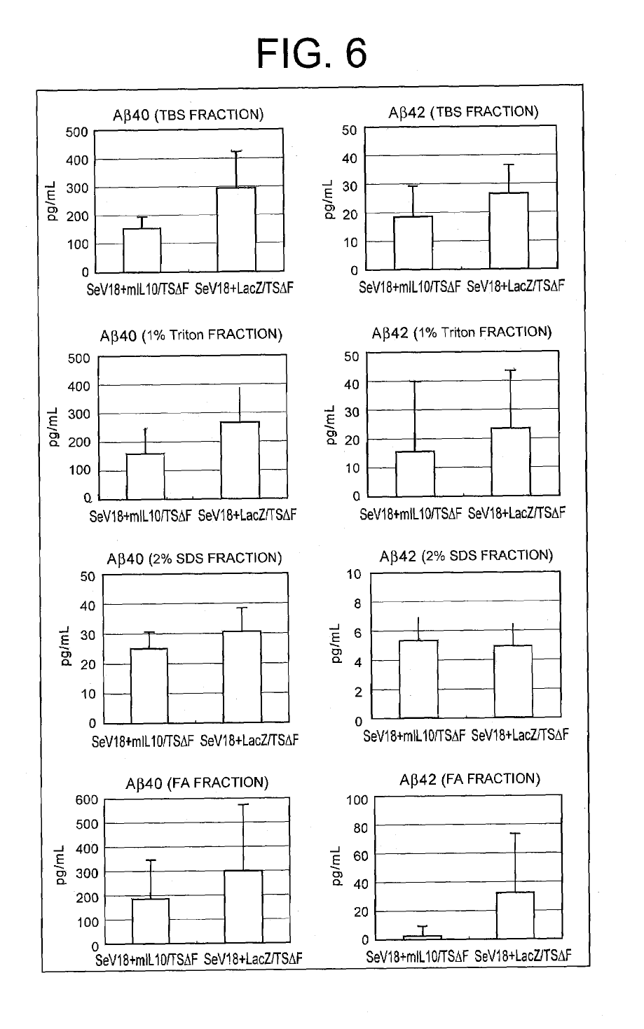

Aβ in the brain tissues was assayed using the Human/Rat β Amyloid ELISA Kit WAKO (Wako Pure Chemical Industries). The result of the assay is shown in FIG. 6. Soluble Aβ40 (TBS fraction and 1% Triton fraction) and insoluble Aβ42 (FA fraction) were decreased in the SeV18+mIL10/TSΔF administration group as compared to the SeV18+LacZ/TSΔF administration group.

### Example 4

### Determination of IL-10 Protein Levels after Administration (Nasal Drop (Nasal) Administration) of SeV18+mIL10/TSΔF

Blood IL-10 levels in normal mice after nasal administration of SeV18+mIL10/TSΔF were determined as described below. Eight-week-old C57BL/6N mice were divided into two groups, each containing six mice. SeV18+mIL10/TSΔF was intranasally (nasally) administered to each group at 5×1 or 5×10⁸ CIU/20 μl/head per animal. Before the administration and three days after the administration, blood was collected from the mice, and plasma was prepared. The plasma IL-10 level was determined using the mouse IL10 ELISA Kit Quantikine (R&D Systems) according to the appended protocol. The mouse plasma was diluted 50-fold using the dilution buffer attached to the kit.

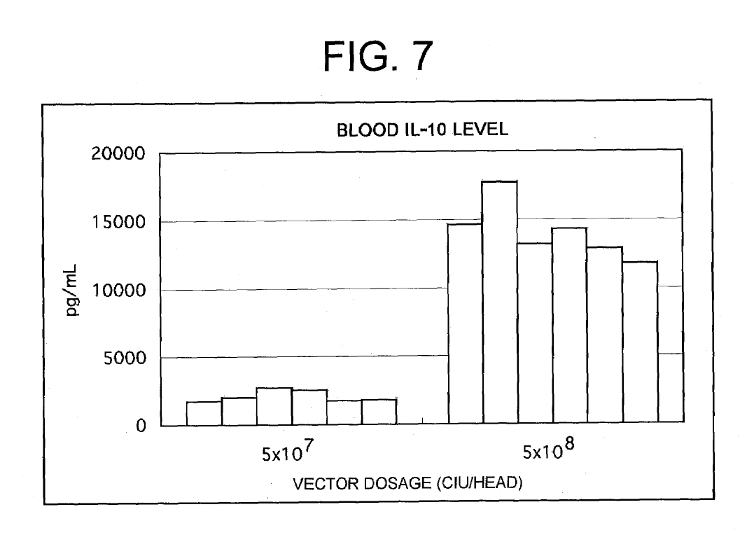

The plasma IL-10 level before administration was lower than or comparable to the detection limit (4 pg/ml). The plasma IL-10 level three days after the administration was increased in a dosage-dependent manner (FIG. 7).

### Example 5

### IL-10 Protein Distribution after Administration (Nasal Drop (Nasal) Administration) of SeV18+mIL10/TSΔF

### (1) Kinetic Measurement of Blood IL-10 Level

20 μl of the SeV18+mIL10/TSΔF vector suspended in DPBS(−) at 5×10⁷ CIU/20 μl or 5×10⁶. CIU/20 μl was nasally administered to six mice in each group of normal mice (C57BL/6N, female). Then, blood was collected from the eye pit using heparin-containing hematocrit tubes at 24-hour intervals up to day 7. After blood collection, plasma was separated using a hematocrit centrifuge and stored at −80° C.

As a control, 20 μl of SeV18+LacZ/TSΔF was nasally administered at 5×10⁷ CIU/20 μl to six mice. The mouse plasma IL-10 levels were determined by ELISA (R&D Systems, Catalog No. M1000), according to the manual instructions.

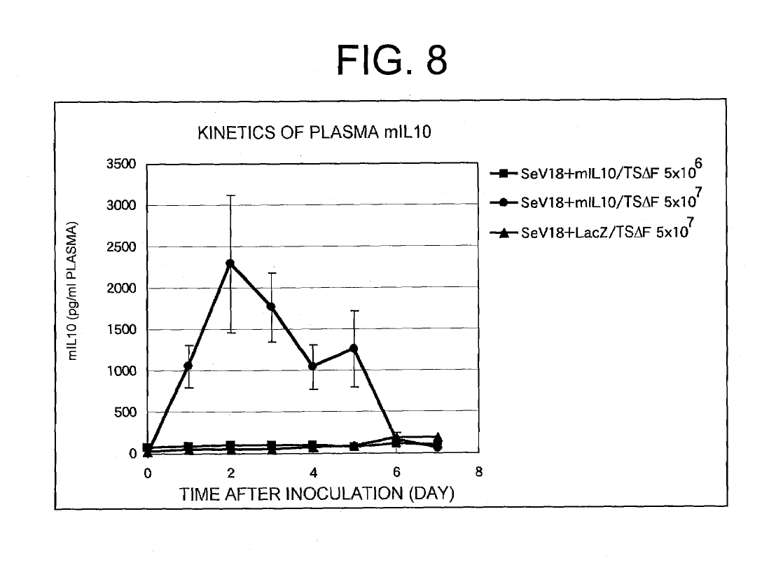

The result shows that AUC (area under the pharmacokinetic curve) was 176,000 pg·h/ml after a single intranasal administration of SeV18+mIL10/TSΔF (5×10⁷ CIU/head) (FIG. 8).

(2) Transfer into the Brain (Concentration in Brain Tissues)

53 μl of SeV18+mIL10/TSΔF suspended in DPBS(−) at 5×10⁸ CIU/53 μl or the same concentration of SeV18+LacZ/TSΔF vector (as a control) was nasally administered to ten animals in each group of normal mice (C57BL/6N, female). As a non-administration control, 53 μl of DPBS(−) was nasally administered to ten mice. The mice were sacrificed three or seven days after administration of the above-described vectors. Hereinafter, samples derived from the tissues of the mice sacrificed on day three and day seven are referred to as “day-3 sample” and “day-7 sample”, respectively. Five mice from each group were anesthetized with ether and underwent thoracotomy. After cutting the right auricle of heart, perfusion was performed for about eight minutes by injecting 30 ml of physiological saline into the left atrium using a 24G-needle syringe. After perfusion, the first cervical vertebra was cut, and then craniotomy was performed to collect the brain. After dividing the brain into two hemispheres, they were further divided into the following three parts: the olfactory bulb, cerebrum, and cerebellum/medulla oblongata, which were rapidly frozen in liquid nitrogen and then stored at −80° C. The five non-perfused mice were anesthetized with Sevofrane and underwent laparotomy. After exsanguination by cutting the caudal vena cava and ventral aorta, craniotomy was performed to collect and store the brains, as described above. From the non-perfused mice, the sites of administration: nasal septum, ethmoid bone, and nasal turbinates including nasal mucosa, were also collected and stored. Each sample was homogenized in the extraction buffer (1% Triton X-100, 50 mM Tris-HCl (pH 7.5), and Complete Protease Inhibitor Cocktail (Roche Diagnostics)) using the glass-Teflon™ homogenizer (750 rpm, 10 strokes) and then the Dremel homogenizer (30,000 rpm, 30 sec). Then, the homogenate was ultra-centrifuged at 100,000 g for one hour. The centrifuged supernatant was collected and used as extract. The extract was stored at −80° C. The mouse IL-10 level in the extract was determined by ELISA (R&D Systems, Catalog No. M1000), according to the manual instructions.

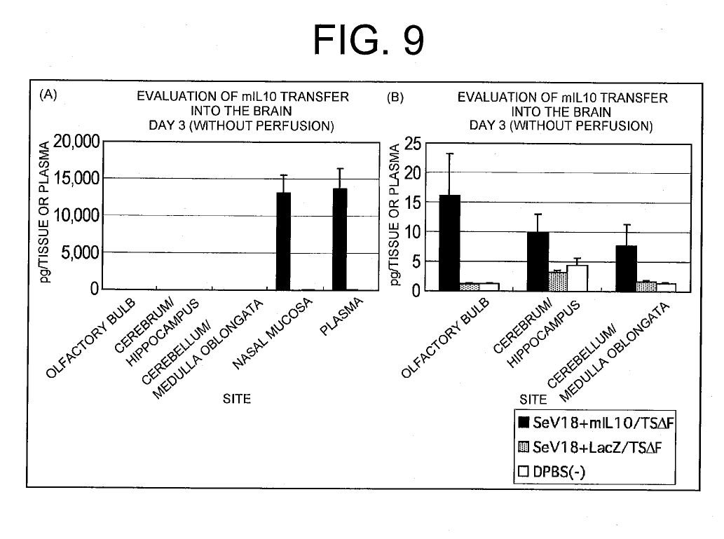

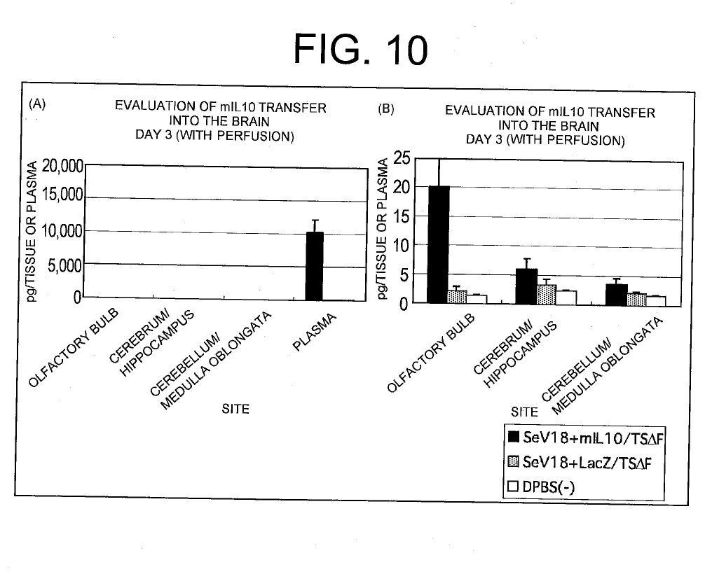

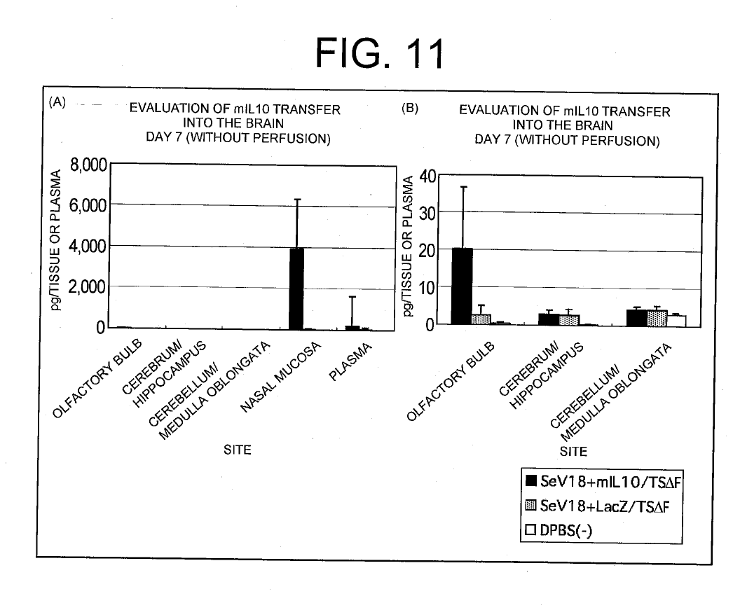

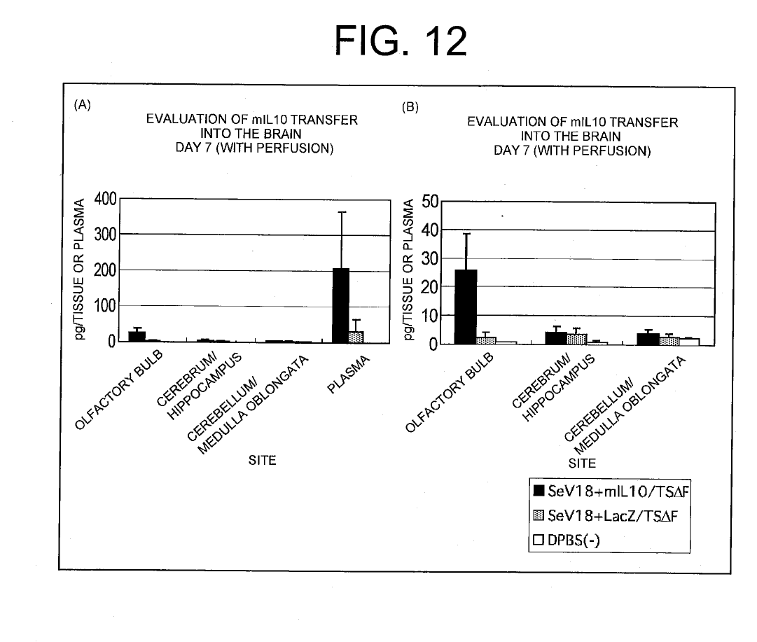

After administration of the above-described vectors, mIL-10 was detected in each tissue of the day-3 sample of the non-perfused group. The mIL-10 level detected in the brain extract of the administered mice was about 0.1% of the expression level in the plasma and nasal mucosa (FIG. 9(B)). Furthermore, in the group where blood was removed by perfusion, IL-10 was also detected in the tissues including the brain (FIG. 10(B)). In the olfactory bulb, the detected mIL-10 level of the day-7 sample of the group where blood was removed by perfusion (FIG. 12(B)) was comparable with that of the day-7 sample of the non-perfused group (FIG. 11(B)).

Furthermore, after administration of the above-described vectors, the detected mIL-10 level in the olfactory bulb of the day-7 sample of the non-perfused group (FIG. 11(B)) was comparable with that of the day-3 sample of the non-perfused group (FIG. 9(B)). In addition, the detected mIL-10 level in the olfactory bulb of the day-7 sample of the group where blood was removed by perfusion (FIG. 12(B)) was comparable with that of the day-3 sample of the group where blood was removed by perfusion (FIG. 10(B)).

Thus, it was demonstrated that mIL-10 expressed from the nasally-administered SeV vector was transferred into tissues of the central nervous system (FIGS. 10(B) and 12(B)).

In particular, the expression level of mIL-10 in the olfactory bulb was confirmed to be constant in the presence or absence of perfusion (FIGS. 10(B) and 12(B)). This suggests that mIL-10 expressed from the intranasally administered SeV vector is surely transferred into the olfactory bulb.

(3) Transfer into the Brain (Concentration in CSF)

106 μl of SeV18+mIL10/TSΔF suspended in DPBS(−) at 1×10⁹ CIU/106 μl or the same concentration of the SeV18+LacZ/TSΔF vector (as a control) was nasally administered to five animals in each group of normal rats (Wistar, female). As a non-administration control, 106 μl of DPBS(−) was nasally administered to five rats. The rats were sacrificed three days after the administration. Under anesthesia, the skin and the muscles of the lateral cervical region were removed to expose the dura mater. A 200-μl pipette tip was inserted into the magna sterna to collect the cerebrospinal fluid (CSF). The CSF was rapidly frozen in liquid nitrogen, and stored at −80° C. The mouse IL-10 level in the CSF was determined by ELISA (R&D Systems, Catalog No. M1000), according to the manual instructions.

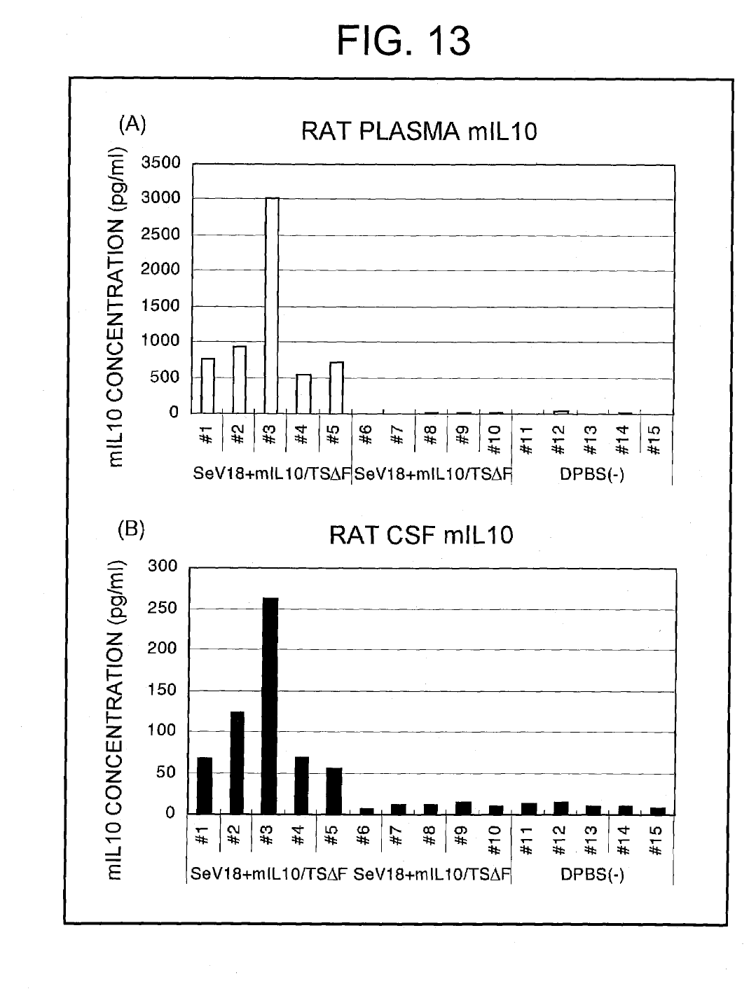

As a result, the mIL-10 concentration detected in the CSF of the administered rats was about one-tenth of that in the plasma (FIG. 13). There was a positive correlation between the mIL-10 concentration in the plasma and that in the rat CSF. This suggests that mIL-10 expressed from the nasally administered SeV vector is centrally transferred.

### Example 6

### Assessment of the Efficacy of the IL-10 Protein (Subcutaneous Administration)

### (1) Kinetic Measurement of Blood IL-10 Level

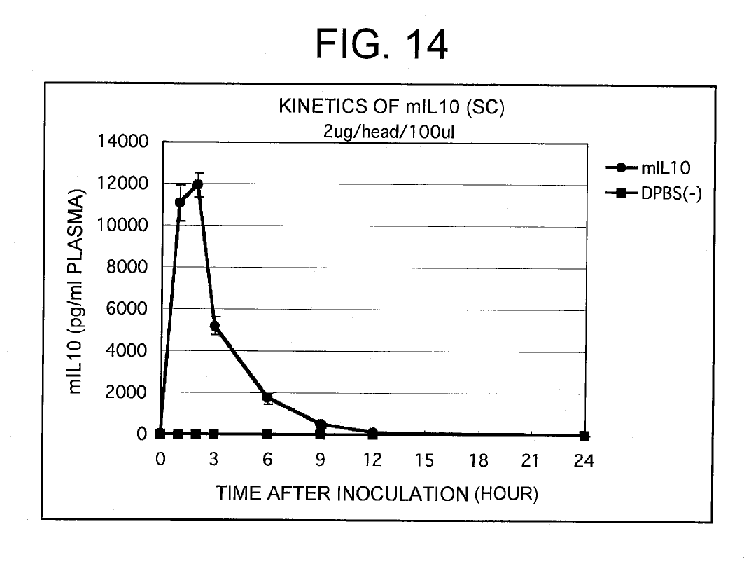

100 μl of recombinant mouse IL-10 (Wako Pure Chemical Industries, Catalog No. M1000091-04691) suspended in DPBS(−) at 2.0 μg/100 μl, or 100 μl of DPBS(−) as a control was subcutaneously administered in the back to three animals in each group of normal mice (C57BL/6N). Blood was collected from the eye pit using heparin-containing hematocrit tubes one, two, three, six, nine, twelve, and 24 hours after the administration. After blood collection, plasma was separated using a hematocrit centrifuge and stored at −80° C. The mouse plasma IL-10 levels were determined by ELISA (R&D Systems, Catalog No. M1000), according to the manual instructions (FIG. 14). The result is as follows: Cₘₐₓ=about 12,000 pg/ml; Tₘₐₓ=about 1.5 hr; t_{1/2}=about 1 hr (initial value). AUC was 40,800 pg·h/ml.

The AUC ratio relative to that of the SeV18+mIL10/TSΔF administration (FIG. 8) was 4.3 (176,000/40,800). This suggests that repeating subcutaneous administration for about four times can result in an AUC equivalent to that achieved by nasal administration of SeV18+mIL10/TSΔF.

### (2) Determination of Blood IL-10 Levels in APP Mice

IL-10 from recombinant mice (Wako Pure Chemical Industries, Code No. 091-04691; http://www.wako-chem.co.jp/siyaku/info/bai/article/cytokine.htm) was suspended in DPBS(−) at 2.0 μg/100 μl. 100 μl of the suspension (hereinafter referred to as “IL-10 suspension”) or 100 μl of DPBS(−) as a control was subcutaneously administered in the back to eight animals in each group of APP mice (Tg2576, female, 13-month-old) every twelve hours over seven days (a total of 14 times). The body weights of the APP transgenic mice used in this assessment were about 20 to 30 g. Blood was collected from the eye pit using heparin-containing hematocrit tubes one hour after administering for the first (day 0), seventh (day 3), and thirteenth (day 6) time. After blood collection, plasma was separated using a hematocrit centrifuge and stored at −80° C. The mouse plasma IL-10 levels were determined by ELISA (R&D Systems, Catalog No. M1000), according to the manual instructions.

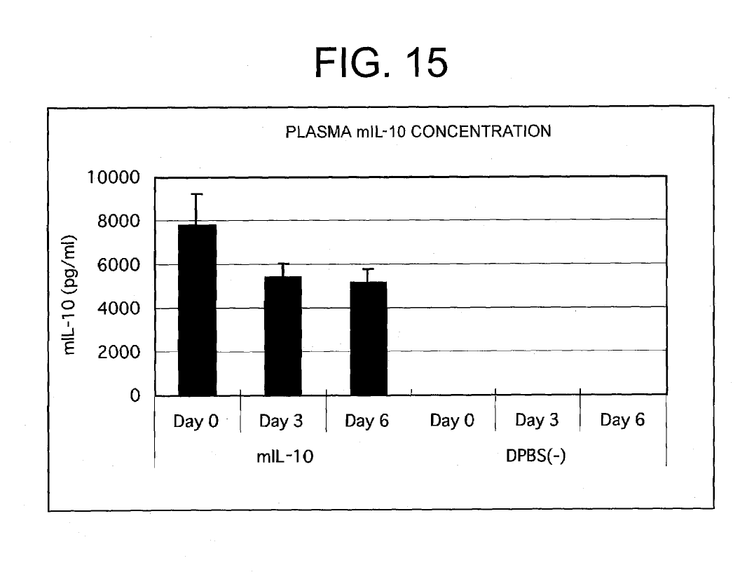

Subcutaneous injection of recombinant mouse IL-10 in the back resulted in a plasma IL-10 concentration of 5000 pg/ml to 8,000 pg/ml one hour after the administration (FIG. 15).

(3) Senile Plaque-Eliminating Effect in APP Mice to which the IL-10 Protein was Administered

The IL-10 protein was subcutaneously administered to mice twice a day for seven consecutive days. The mice were dissected four or eight weeks after the administration. Brain tissue sections can be prepared from regions such as the cortex of frontal lobe, parietal lobe, and hippocampus. The experiment described below can be conducted using the cryosections. To detect the Aβ protein or senile plaques in the tissues, the sections were treated with 70% formic acid, and the endogenous peroxidases were inactivated with 5% H₂O₂. After reaction with a rabbit anti-pan-Aβ antibody (1000-fold dilution), a peroxidase-labeled secondary antibody was added and then DAB staining was performed. To evaluate the degree of senile plaques (Aβ-accumulated portions) in each region, the area of senile plaques was determined semi-quantitatively or by viewing tissue section images under a light microscope. The area ratio of senile plaques in each site tested was calculated.

Specifically, the effect of IL-10 suspension was assessed using 13-month-old female APP transgenic mice (Tg2576). The body weights of the APP transgenic mice used in this assessment were about 20 to 30 g. The mice were divided into two groups, each containing 15 mice; one was the treatment group and the other was the control group. 2 μg/100 μl of mouse IL-10 suspension, or 100 μl of DPBS(−) was subcutaneously administered to an animal in the treatment group or the control group, respectively, twice a day at twelve-hour intervals. Four weeks after the treatment was terminated, blood was collected from eight mice from the IL-10 suspension-administered group and seven mice from the DPBS(−)-administered group, and then the mice were dissected. The brain with the olfactory bulb was vertically divided into the right and left halves. One was rapidly frozen and stored for biochemical measurement, and the other was embedded in an OTC compound and frozen in dry ice for histopathological examination. Histopathological samples were prepared as vertical sections containing the olfactory bulb at 1 mm from its midline. The cryosections were prepared, fixed with formalin for a short period, stained with hematoxylin and eosin for standard histopathological examination, and then observed under a microscope. Immunostaining with an anti-Aβ antibody (4G8) was performed to detect the Aβ protein and senile plaques in the tissues. Alternatively, Iba-1 immunostaining was performed to stain microglia and macrophages.

Samples stained with the anti-Aβ antibody were divided into the following four parts: the olfactory bulb, cerebral neocortex, hippocampus, and brain stem/cerebellum. The quantity of senile plaques and blood vessels positive for anti-Aβ antibody staining present in each region were evaluated under a light microscope. In general, senile plaque deposition is not observed in the brain stem/cerebellum region. Thus, senile plaques in the olfactory bulb, cerebral neocortex, and hippocampus observed under a light microscope were classified into three categories based on the size (large, middle, and small), and a score was assigned to each category (large: nine points; middle: three points; small: one point). The degree of senile plaques was semi-quantitatively assessed from the total score. Furthermore, the effect of IL-10 was statistically analyzed using the total score for each individual. The areas of senile plaques in the olfactory bulb, cerebral neocortex, and hippocampus were quantified by image analysis software (NIH Image, Japanese Edition) using recorded images with the same magnification.

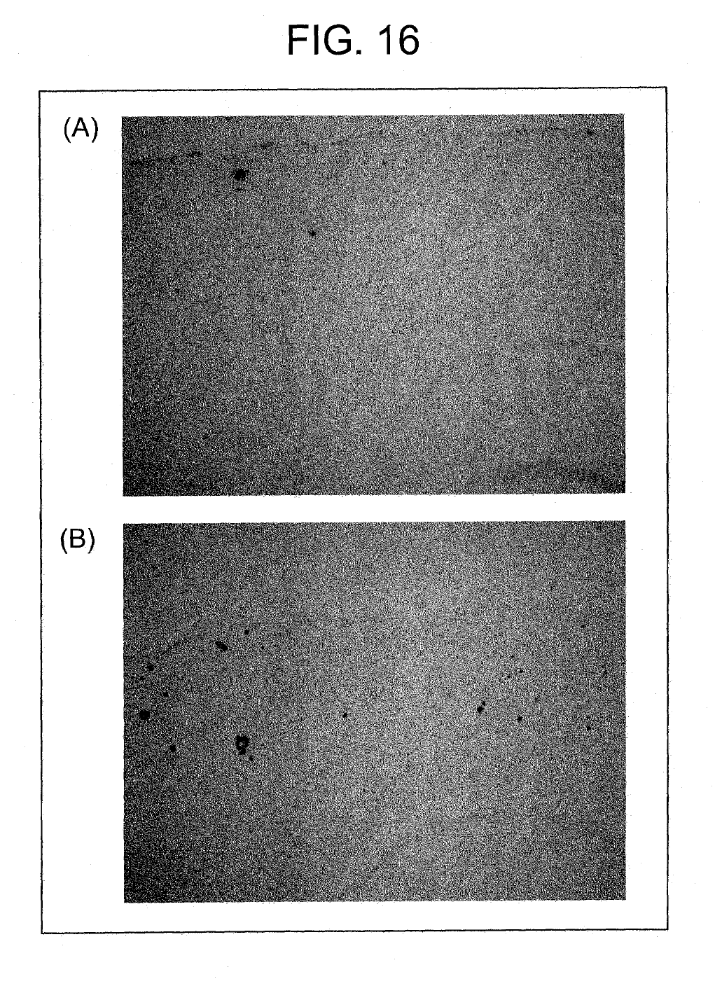

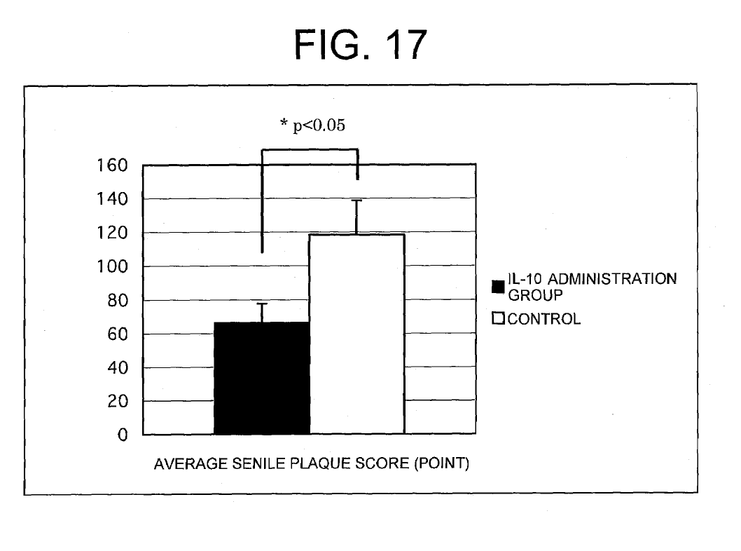

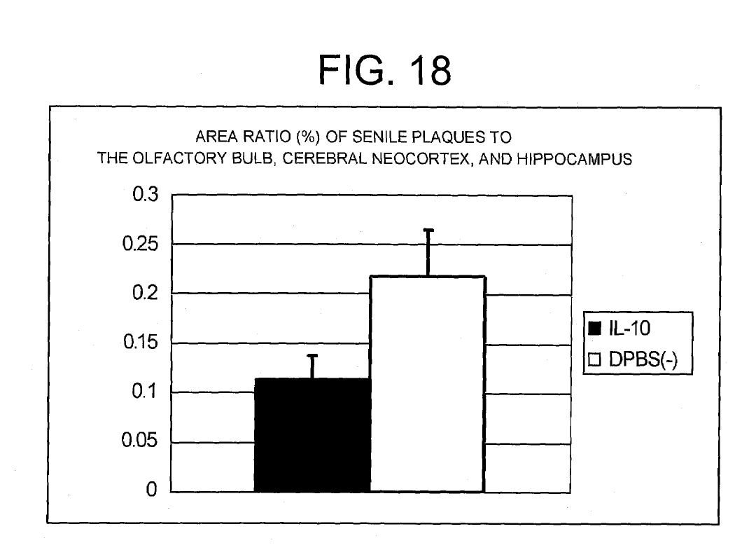

An example of the stained sections is shown in FIG. 16 (parietal lobe of cerebral neocortex and hippocampus). The result of semi-quantitative determination of senile plaques is shown in FIG. 17, and the result of quantifying the area of senile plaques is shown in FIG. 18. In both groups, senile plaque formation was clearly observed in the olfactory bulb, cerebral neocortex, and hippocampus. The number of senile plaques in the olfactory bulb, cerebral neocortex, and hippocampus four weeks after the treatment is clearly reduced (about 50%) in the IL-10 suspension-administered group, as compared to the DPBS(−)-administered control group (FIG. 16). As a result of semi-quantitative determination, the difference was statistically significant (p<0.05) (FIG. 17). Furthermore, the result of quantifying the area of senile plaques demonstrated that the degree of senile plaques tended to decrease (about 50%) in the mIL-10 administration group as compared to the PBS control group (p=0.06) (FIG. 18). This suggests that the elimination of senile plaques or Aβ deposits in the brain by activated microglia or macrophages was enhanced in the IL-10 suspension-administered group.

SeV18+mIL10/TSΔF and SeV18+LacZ/TSΔF were used in the above Examples; however, the vectors of the present invention are not limited thereto. For example, effects equivalent to those described in the Examples can be expected when non-F gene-deficient SeV vectors, F gene-deficient but non-temperature-sensitive SeV vectors, or such, that carry an anti-inflammatory cytokine gene such as IL-10, are used.

### INDUSTRIAL APPLICABILITY

The present invention provides therapeutic agents for Alzheimer's disease, which comprise an anti-inflammatory cytokine, or a vector expressing an anti-inflammatory cytokine, such as a negative-strand RNA viral vector encoding an anti-inflammatory cytokine, and gene therapy methods for Alzheimer's disease using the cytokine or vector. The methods of the present invention are novel therapeutic means that can be used to substitute for or in combination with other therapeutic methods for Alzheimer's disease.

1. A pharmaceutical composition for treating or preventing Alzheimer's disease, wherein the composition comprises
a negative-strand RNA viral vector carrying a gene encoding an anti-inflammatory cytokine or a partial peptide thereof, or
an anti-inflammatory cytokine or a partial peptide thereof.

2. The composition of claim 1, wherein the composition comprises a negative-strand RNA viral vector carrying a gene encoding an anti-inflammatory cytokine or a partial peptide thereof.

3. The composition of claim 1, wherein the composition comprises an anti-inflammatory cytokine or a partial peptide thereof.

4. The composition of claim 1 or 2, wherein the negative-strand RNA viral vector is a paramyxovirus vector.

5. The composition of claim 1 or 2, wherein the negative-strand RNA viral vector is a Sendai virus vector.

6. The composition of any one of claims 1 to 5, wherein the anti-inflammatory cytokine is selected from the group consisting of interleukin-4, interleukin-10, interleukin-13, and partial peptides thereof.

7. The composition of any one of claims 1 to 6, wherein the composition is used for nasal administration.

8. A negative-strand RNA viral vector carrying a gene for an anti-inflammatory cytokine or a partial peptide thereof, wherein the vector is used for treating Alzheimer's disease or developing a therapeutic agent for Alzheimer's disease.

9. An anti-inflammatory cytokine protein, wherein the protein is used for treating Alzheimer's disease or developing a therapeutic agent for Alzheimer's disease.

10. The vector of claim 8, wherein the negative-strand RNA viral vector is a paramyxovirus vector.

11. The vector of claim 8, wherein the negative-strand RNA viral vector is a Sendai virus vector.

12. The vector of any one of claims 8, 10, and 11, wherein the anti-inflammatory cytokine is selected from the group consisting of interleukin-4, interleukin-10, interleukin-13, and partial peptides thereof.

13. A method for treating or preventing Alzheimer's disease, wherein the method comprises the step of administering
a negative-strand RNA viral vector carrying a gene encoding an anti-inflammatory cytokine or a partial peptide thereof, or
an anti-inflammatory cytokine or a partial peptide thereof.

14. The method of claim 13, wherein the administration is nasal administration.


SEQUENCE LISTING (19 sequences)

SEQ ID NO: 1 - RNA, 10 nt, Artificial
  gene start (S) sequence
cwuuvwcccu

SEQ ID NO: 2 - RNA, 10 nt, Artificial
  gene start (S) sequence
cuuugacccu

SEQ ID NO: 3 - RNA, 10 nt, Artificial
  gene start (S) sequence
cauucacccu

SEQ ID NO: 4 - RNA, 10 nt, Artificial
  gene start (S) sequence
cuuucacccu

SEQ ID NO: 5 - DNA, 10 bp, Artificial
  gene start (S) sequence
agggtcaaag

SEQ ID NO: 6 - DNA, 10 bp, Artificial
  gene start (S) sequence
agggtgaatg

SEQ ID NO: 7 - DNA, 10 bp, Artificial
  gene start (S) sequence
agggtgaaag

SEQ ID NO: 8 - RNA, 9 nt, Artificial
  gene end (E) sequence
uuuuucuua

SEQ ID NO: 9 - DNA, 9 bp, Artificial
  gene end (E) sequence
taagaaaaa

SEQ ID NO: 10 - protein, 43 aa, Homo sapiens
DAEFRHDSGYEVHHQKLVFFAEDVGSNKGAIIGLMVGGVVIAT

SEQ ID NO: 11 - DNA, 537 bp, Mus musculus
  CDS (1)..(534)
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 12 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 13 - DNA, 51 bp, Artificial
  a PCR primer
acttgcggccgccaaagttcaatgcctggctcagcactgctatgctgcctg

SEQ ID NO: 14 - DNA, 79 bp, Artificial
  a PCR primer
atccgcggccgcgatgaactttcaccctaagtttttcttactacggttagcttttcattttgatcatcatgtatgcttc

SEQ ID NO: 15 - DNA, 596 bp, Artificial
  a Not I fragment encoding mouse IL-10
gcggccgccaaagttcaatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaccgtagtaagaaaaacttagggtgaaagttcatcgcggccgc

SEQ ID NO: 16 - RNA, 14172 nt, Artificial
  SeV18+mIL10/dF (genomic RNA)
accagacaagaguuuaagagauauguauccuuuuaaauuuucuugucuucuuguaaguuuuucuuacuauugucauauggacaaguccaagacuuccagguaccgcggagcuucgaucguucugcacgauagggacuaauuauuacgagcugucauauggcucgauaucaccuagugauccaucaucaaucacggucguguauucauuuugccuggccccgaacaucuugacugccccuaaaaucuucaucaaaaucuuuauuucuuuggugaggaaucuauacguuauacuauguauaauauccucaaaccugucuaauaaaguuuuugugauaacccucagguuccugauuucacgggaugauaaugaaacuauucccaauugaagucuugcuucaaacuucuggucagggaaugacccaguuaccaaucuuguggacauagauaaagauauccaagauagcacaagucuucuagaaauugucuucaacuugccugaaucucucacaggauacaggucauacuuaccaguuaguuuaagccuuuuaucugacucaguuauucuagcccauucgaagauuguauccuucaaaacccuguugaaagcuaucaugcaguuguguguaucagcuauguucaugguggacaagaaaucucugcucaauuuauacaaguuugguucaaagccaaacgucugcagugcuugaugguaaggucuaagcaucccugaugaagagcuuccuucucucaauucccgaguaacccauucgugagccuuugccuucucuauuaagauccacuuuucuaucuugaugcuaucuucuuuugacaaagggaggagacuagcuaacacugucuugcuguccucuauaaugucagauuuggggugccucgauaggagauacaucucuguggaagcaggguuagauguuuuaagcacuauuagguuaaccucgucccaguaucucagauauaggcugagcugccugguccaauccgugcccagccugggagcaaucuugcuuauaagcacaacgucucgaucccccaccagauacgcgauccggauuacacuguaaugcucaugcaguacaacuugaucauccuuaugaucuccucccuccaugucacaguggacuaggccuaucgagcuauucuguaauucauuccaaaucaaagccucacacucaucauucccaauccaugucgagccaggauucccguugaauaacacuuuaacucuuugacccagacuaguaacauuguuuaauuucuuucccacuagugccaccucagcaggauauauauuuaacucucucugcccauugacaucacaagaguauaccccugaguuauaauaguugaugcaugggccaagaguagcgucauaacaggaaagcauggccccagcuccuuccccuaaauauagccuaucuuuauccuugucaacuaaggggcucaauagguagguaaguucaagugcuuucaagcagcuaguacuguugaugccaaagagccucagcuggugagauagauaucuuccaucgaaugguaacgucagaccaacaccugaucugccuaucucaccucuugugaucuccuuguacccccggagacgcaguaccugugacuuggauacucucaacccccaaaaaggggcucuagucugaugucccaaaggaauauucuguuguauuucugccgcgauaccaucuaacagugcauuaucugccucgggaucccaaucuucuaagacuucagggacuccuauaccucuuguccuuaacacuuuuauagaugacuuccggauauaggucaaaguagaugggaauacuuugaugacuaggccagucaacugacuguaccucaguaccgggucaucaagaaaugugaguuccagguaacucuuuagugacucgagccucucuagagaguucauugauucuaaucuuggcccaucccuagauaucucugccaagcugcauagguaugcaagaugucuugccaagaaagaggaccuccucaugucggccacaucugggucauugucacagauaaagaucucaagcgguacacccccuugccaaucgugcaugaauagauccacagaauauucacagacagagagggccaagaguaucuuauccugauuugagagguuaggcccauacacagguuccacgacaccugcauuccagaaucguuugaagauuuugggaugagauagagcauuagauaagacuuuuaaaacugcgugggagguaucuuuaagaauccggacuacauguccccauauuucuucccuuccucugauguuuaagccguagagugaguaugcaaacugauugacuagaauacccccgaacguugagcaaaauaaaggaacaucaaucaccauaaacucaguaaucaagcuguugacaucaucgucauuuacuagugcgaucaucucuuuuaaguugucucuaucuaauugagacauuguaucagcuaucgucauugcaguacagauacugguugcucugauaacuucaucaucugaccaauaagucaugucaacuguguguacaacaucucugaccuugcuaaauagcucaagguccacauccuugaguggaucaggaucauagaucaauuuauuguucucuuguguaaucucuaaaucuaauguggaccuuggggggauauucgccuccuguggggacuccauuauacagcacccguuauuaagauguaagugcaauaucaguggcuucccuaaggaaccuuucuuauaucucauauugaacucgaacaagcuuagcccaguuagcauaaucugcugauacacgagauuaguauccuucgacuccccugcuucuuugagugccauguuaucauuugauauuguuaugaaccgacuugcacggacuaguguugcacuagagaacuucaucuggguugccguaucuuucaaccuaugagauagauuaguggagguugaaacaggagucagcagcuuuagauucucuaagcucagauuagcucuuguuugggcuauaagagcggcuuccauccacgauaucucaucagucccguaggcccacguauacaccauagcuauccggauggccgccuuugcggguuugcuuagauuucuuacauacccgaguugggcuuccgaccuuucaucaguggcugauccaaaauaggggauucuuauagccggacauccguuuguaagcguguccaggucuauauugucaggaagauagaaccauguauagauggggucugcuccuucagaccugcaaagcuugcacaccucugaaccuucgauaaauauuccccucaagagcucuaaagggucugguguuucuagcccauguaugggucucccguaagucagguggauccacauuuucugccuuagaccgacagcuagcucaacugaacacauauacucauauucgauguugucuuucaccgguuuccugagaguucuagucagugucucguacugcaauagaucauaauugacaagccuccucaauaucccauaugauaauccuccuuuccuaacgcuggcucucacuagagacuuggucguaucaagcaucccugcaaucgccucccuaacuccaguuaaggaauuacccaggaucucaugagccacucucggcaggaugacuuuccgguccauaaggaacgaggccagguugagauccucuucuccacuagucucggugaagagaccagacaguagaggauucggggauuccugcagcacagaucuagcagugauauucuuuauaaucguaguuauacucugagaaugcgggagguuacacgaauaagggucugaagcccaaucuagaaaacuagagucaccggguucuugauucaugacccuguauaauaccugcuugucuaacagauccgcucugaugaaucuuuugagaucagcuagggcugcuacugcggggucuccaauauuucuaacaaagcaucuagauguagacauguaguugaauccuccaacauuugcuggaaucaacacugcacaucucagccaauucuuacccuuaaaguauugaucucuuacggucgggcugauaguuggauuuauagucaucccuagugauaugcacaccugcugacaggucuuauacaacgcaaugcaguagccuaguauaggagaauacccauuuucgauagcuuuugcuauggauguugagauguucgaacaagcagaucuguuuucaucuaccagugucucggaccagaauacacacuuggucaaggcuuucaggcacugugguaaaaucuucccaucauaguauauccuuuuacuauagacaaacaucuugcuacuaaugauggucucguucaauuuuagcucgugcccuacaucaaacaugacgugucuuagagcaccgaaauauuuggugaucuccucauagacaugauuuuucuucugcuuguaagucugagcuacagguacucuugaugucacggcuauagcuugauugucacccugaaccauugcagagacccugacacccacucucacagcugcuagguggauugcacugauugagauuaagguccacagcuucuggcaguaaccuucuaugcccccccuaggauuauguaugaaaaugccagagucugcaugauccuggaguugucgaugcauccggucggcgacuggacaguaaggaucuccaacauauauuguacaccuuucaaggacuggaugcauccaguuaaagaaggucuugaagccaaauaucucguugcaucucugaccaaacaaugcaguacucucaaaucuccaguuuaagcaguauuucuugaggucuguugugaggaagcaacuuaacguuucauagccgucuguugaugaaucuguugccuugaauucaugucuggaccucuucuuuucgucccaguaccccccagaguucuuauuuuccaugccuucguuucucuucucugaugauuuagaguuauuguacacugaaucaguccuggggacgccugagacagaaagaguagucaaucuuuuaaguaggucuaucucuccuuuaaccaucccauuuucgcugaauagcucuccuauuccuuuagccaguagugucucugccagcaccuguacggcucgcaucuuauaagucauuuuugcgaauagacgacccucuugcuugaucucuuucucuuugagacuguacgagauguugaacuccucgucuuucaaccaaucuccugacuccacauaauugauaauuucuucuggguugaaauucucaucauuuaugaacacuucaauaagccgccgggucucuucagacucuggggcuuuauaguacagauuacuauccggguauacagagucccaugccuccuuccugggggauagugcuuugucuuucauauauauugugagaucuucaucuaguugugguucuauaaacuuccgaaacuugaagccuaugaaacuuguauaguugucuacagcacauucauaagagauugccguauuggacccuugagcguuccuuaguucuagacacacgugaucagggaagucacaggggggccacuguccgccaugccucucucuauacccauuuaugaugauagugcaaaaaacugcaugacacucguauagggucuuaagcuuuauugccuuuugugcauacauaugggcccuuaccuugucggcggcagugacagccucuaagcugggguggccaaauguccuaaagaaggaaaagaucucugcuuucucaucaauagagguuccauggaaaauggcgaguaacgacuccacaauagugucugcuucagcaucuguguacacgucucuacuuguuaaaacagucuguagcucugucaacacaugccucauaaaugccccacguagagguauaacaggaucauuuaguuguaugagagcaagugauaggggcuccaauagugcgaugacauuguauauuuccucuccaagacuugagaagagggaauccacuaguucccauaauuccucaccuuugcuuguuaucccaauggacuucuuaucuagaugcccugcagcagacauauuccaccuuccuucuacaacaucacaauacaucaagaccagcucagggguuaggauauacccugucaaugucaacuuguucaguaucaugacaagaucuccguauguuacuagaguguaugauuugcauucuaggagguuaugugaauuagagguaucgagggguccccccggucuggucuucugcauccaccgcaugucauauuugaugcugaaccaaguuaggaauggccuauaccaucuauuccugcuguacuuaucaguuaucuccgggauggugccgauauccugcaacggaucguaccccucucuuccuucuauauugccuaguugcuugaagauauucaaccaaagauccuggaacccacuagauaacucccuggucagccgauccgagaccgcgaagacggaucggauuuugucacauaucucugguauaucaagccuaaguaauuccugagaguagguuggguacgguucaaacguguaucggucuaaauccuuuauugugcguugaagagccuuacccagagaccugaucuuaauuugacggggggacaauccuccguuccuaauuuugugcuuuguaauauuuauuaugcugucguccuucagucuguagggcugguucacaucuaacaagacgugcaacugugcuaucuucccccugacuaugggagaguucagguggcauucuggauagaguaugucagaaggguuuugggaggacuccugcccauccaugaccuauggcaagcuucccauucacccuggguuuuucuuaauacugugagagacguaagagacugagagauauuguggauguucggagaugacucuagugucagcaaggccgacaagccugcuagucaguuaaauuuaagacucggccuugcauaauuuagggaugcuagucuuaaagagcaucggcuguaagguauucaggcucuucugauugaucucgaugaugugaaagcaguagccuuuaccaaaaugcgugauacacgaugucgugguauaugcagccucuaauugaacauccuuuauccuuaacauauuuauaauguuaguaguguuagaauacaugauuguuggguugacacgcgauguauuggcauauagcgugacgguagcgacguuagcugcaucaggggacaauggauaagcaucaguguauacgccugauaugcauuccuucggacacuuauuguaccaauugcacucuuuauuuccuggucuagacaaggcuucaugagguguccaguugauagucaaaggguggcugacaucaaguacuccuaucugcaguugagagugccagccugaugaucuuguauagauguacacccgaucacccaauuuuaauaaucuaccuuccgccccgagauaguuuugagugauuggaaugguugugacucuuaucuuuggccucucugagagauagucauugaccuggaugaucacgcugaccaccuguuucccuccuagccauguaauuuucagagccucauugcaugugucuugcgacaccuguuggcauccuuggguccuacauuuuguaucacccugcagaggggugguuaguccaccauacccaagaaauaucaaugagccuucuguugcaaugccguugccuacacugggguauagugcagagaacgggugaucaagaucuaccucgcuguugcgauaccggugagacuuaguucucccuuugagauccaggacaucaaggaccagauccucaauaccaucacuagaguagucgguucuuucgucuacagucggcauggagcaaagcugauaaccccuagucccgguugccaccacagagcaugauuuccgauugucguugaugucauaagugugggacacuacgggguuaagaucagggaacauaucugaauugagugauauguacccuagcugcaggaccugauaugauuucccuaugucagcacaaccuuguguaaugagauuugaugaauaggcauagauugccucgccaauugagagugaagggagccuaacacauccagagaucguuguagaaccagauaacaagcucggaccaggcagcaaugagauuucaggaucugagcuaagauacgguucuccgacagggcaucuccagaaacuauguggcucaaguggggcaauuccaucggcaugguggacugcgaucguacucucacagugcugagugagcucuugucugcugcacgacuuaucaaucaucuggaugacaucccugcuguuuuuguucaacaagacugggauuccgguuugcacagagcucugaauguugacagcccuugcuauaaccucuugccuuauuagacugguaagugacucuuucaccucccugcugcucauguucaaugccucuacagucauugaguacucuuucauacuauacccuugucuagcagaaauuaugauacagaugaucacuguggcaauugacaaagcccacugggugaaugagagaaucagcaaccaugugucggcuuuacuugaccucucccaaccugaugcugguuuuguggugcuaccacuaggagaaguagaccaguacgagucacguuugccccuaucaccauccaugaucugugcuuguaccagguggagaguuuugcaaccaagcacucacaagggacuuuaucccuaaguuuuucuuauuuaagacaaggagugacccgguggcuccgacugccagguggugucugcuuggggcauugucgcaggucucugaugggugcacauuuacagcuuucugauccuuccgauguucuuagccacaacauuaggguaguagcggaaaucacgagggauggccgguuggaacaccgcaucgacgccugugauuucuacagaugccgcccaaaucaccauguucauauggggauucacauccauuaaugggaagcagacugcccucuuccaugcgagcugacucaugaaugucuuagauagugucccauuaaccugaacauggaagcuuauaccgccgauuaacccaagugagaaaaucagccgcauucucucaaucuugcucuugcaguacucaacagaguauaucuucccgaccuuucuccugaucaacccgaggugcaccauaaaauugagcuuuuucuccccuugaucaucaaguacugggaguacccccuuuuguucuguggagaucccggucuugagugucaccaguaaauuaacagauauagaguugggcaaugcaaggucugcaagggucuuugggaucuuggcuauggugauugccccuagagaugucccauugacaaacaccacucugagucuuauguccuuguccacagggaggcauuggggagcuagugcgaccuuguuugcauuaaauaucauucccugucucagccugccugaccaugguaggaguggagcaccaaucgaauccaccauguauacgaucaucucuccugcucgaacaguccuccucaccguaauucugagaucggugcaggccuuuaagaguuccugaucagucccguaguauuuggccacaccuauggguaacgacccggagccgcauauugaguagcuggucggcucugucaagucagauacgcucccuagauugguuguuuguuucggugucucaaagaaacccaagagcaauaaaucuagguaucucacuccauguuuaggagggucuccuaccuugacaauccugaugugggggauggcuuucuuauccggaccaguucucagaggcaggggcuccacaguaccguuauccucauaugagaacuuagggaaucuauagauaucugccauugcgccguguuaggugaaauuucuuucacccuaaguuuuucuuaaucuuuacuggcugaugcugaugguagauuggaucucucuaugacugaggaugguaggaugccucacccgggaucuaguuggucagugacucuauguccucuucuacgaguuccaugacugccuuaaccucuuggucugucuugcacuuggauaaugauuucacauaugcuacuuucucagcucugcuuaggggacugcucucuaugacgagccugagagagugcauugugggcuucucuuuggaggggaggagacgugaugcguuugaggcccuggguucugugucccucucuugguacaccggguugcggaucucaucucuaaauucauccucucggauuagguccgguuuguacuucauaucuucuagggucuccauagaugggucaaaccugguagccuuagucuuguucucuuuugauuuugcaaaaacggagggggaccuuguaagggagucuguguugucagucuugccaccucuaucugugaugauaugaaguguagauagguuggacaucagcaaugaguucuguucuuucugauacucagagaaucucuuguaaauaucccggaaugauuccacgcucucuuggaucuguuugagcaguuguuuguucucaucuaccuuacgagcggaagauuucucggcagaaaggaucaggccgcauacauugaaugucaucucugcauaguuugcagacuuuagggcacgucuugcaaacacauaacucgcgucucgggaugauucgaauucuugagcagacuggauuacaccaagacucgucaacaauguagccaucucuuucauagaugauguguucucuccuaugcccuuuuuuguugagucggucucuagacccggguggauugggcggccguuggcuggacaaucugagacagagcgggucccuauuggugguuuccggucuuugacccugacggcggggguguccccagaguugaugugcucauccuguguagauggggguuuuccugguggugacccugugcuguuguaacgauucagcgguggggaccgggugccaggcacgguugcuggaguaagagguuuggacccacuguugguaggucuucuuuuguuccuccguagcacagccucuucaaguucggggcuaggaaucaccaggaccccaguuacucuugcacuaugugagcugccaggcuccaugcuucuuccauuauuacuugcuccaccuucuccuucaucagguagggauguacuuccucguaccucuucuggaaguccuucagcuuggucuucuccccucuuaucagggugcgcagccaucucucuguuuucaucuucaauaccugaucucggauauccucucucauuuggaggauuuucaaggauuccggagucuccuccaucgcccagauccugagauacagaguuuguaccaguucuucccccaaaggcccgguguauauuuuguuuaucaagguuuccagcaugugcuucugccucuggcuugcuuguucucccagagacucuacucuccucaccugaucgauuaucuugggucgacgguguugagacuucuccuucgcccucacuuuuggcucuaugagcagagccugguccuuggggaguguugaugguguuguggagccagcuucuguccccuccgaugucaguugguucacucgacaggacagcaucgaggaauccgauaacauccgagagcgacucucguccuccuggcgccucccucucaacuucagaaucuucuuuaagaaugaaggcaucuugauccaugcgguaaguguagccgaagccguggcugucucgacugcuggguguggucggugggguuggguguggccuugccugagccgaucgguggaugaacuuucacccuaaguuuuucuuacuacggaucaaguaccuugaagccucguaugauccuagauuccuccuaucccagcuacugcugcggcaucgucaucuucgucaugaucgacaccguuauugcggccuucaucuccauggguugcagaauccucuugccgucucucugcgagucucauggcuauucuucucucuaugucugauacauccucaucauugguuuccuccucuaaccguucagccccauguagugugacaaaguggccaccacucaccugacgugcccaucuuucaccacuaucuccaccccaaccccuagcguccugguccgcaugggcuuuuguuucuaggucgaugucggcauugucuagagcuaccucaauugcaccaccgccuguugguuugugguaagcaccauccccaccggacaaguuugccagaugaugucugagccuccccuuggcuguauccgucacuccuaacucaucuuccaaggcacugcugaucuucgauucagcauccuuugccacggcuuguccuaguaagaacauuuccauaucaagguauguccucccugugacguacugcugcauugccuuguucuguacgacggcgacucccauggcguaacuccauagugcaggauaauugccuggagcaaauucaccaugaacaggguccuugaggauacagauaaagggagcucuggggccuuuugacagguaggugucuaugaggcuucuaagcuuauuaauaucgggccucagguuugacaacguuagagcugccaucuuuguuuccaccccauauuuaauaguguucaugaaggaagccagcccugcaucucggauguaguucccaacgaucuggauguucuucucuaauguggugagaucagaucuugcaguauucauagucacaagggucucaaccaugagagauacaaggcuuugcugagaucucauaaccgagccuauccccucaacugucuccccagugaaaacuaaggcaccuuucacggugccgucuugucugaacgccucuaaccuguugaagaacccuuuccuuaagccggcgcugcuugugauggccuucaccagcacaauccagacuuggacaauuauugcuccuaggcaugcaggauacccauagauuugaaggagugugucagggucugcagcaucccguugacccuggaagagugggcucuuguugaccauagguccaaacagccauucugugguccucucauauuccauaucucucgucuucacaaugaauccgucugucuucguccucuuagggucuuucucuauguuguagaucacauauuugacaucggcguuuacuccguuuguugucaaguacaauucuggacuacuguaagccauggcaagcagagagacgaggaaccccccucucugagagugcugcuuaucuguguccaaugagugagcuaggaagguaguugcaaugaauaacuugucugcaucaucagucacacuugggccuaguacgaacacugagacugugcuccucuggccggggauaacagcaccuccucccgacuuauuaauacuuucgcuccuccuagagcuaaauguaucgaaggugcucaacaacccggccaucgugaagaucugcggccgcgaugaacuuucacccuaaguuuuucuuacuacgguuagcuuuucauuuugaucaucauguaugcuucuaugcaguugaugaagaugucaaauucauucauggccuuguagacaccuuggucuuggagcuuauuaaaaucacucuucaccugcuccacugccuugcucuuauuuucacaggggagaaaucgaugacagcgccucagccgcauccugagggucuucagcuucucacccagggaauucaaaugcuccuugauuucugggccaugcuucucugccuggggcaucacuucuaccagguaaaacuggaucauuuccgauaaggcuuggcaacccaaguaacccuuaaaguccugcauuaaggagucgguuagcaguauguuguccagcugguccuuuguuugaaagaaagucuucaccuggcugaaggcaguccgcagcucuaggagcauguggcucuggccgacugggaagugggugcaguuauugucuucccggcuguacuggccccugcugauccucaugccagucaguaagagcaggcagcauagcagugcugagccaggcauugaacuuuggcggccgcgugaacuuuggcagcaaagcaaagggucuggaaccugcuccucaggguggauacuuugacccuaaaauccuguauaacuucauuacauaucccauacauguuuuuucucuuguuuggu

SEQ ID NO: 17 - RNA, 14172 nt, Artificial
  SeV18+mIL10/dF (antigenomic RNA)
accaaacaagagaaaaaacauguaugggauauguaaugaaguuauacaggauuuuagggucaaaguauccacccugaggagcagguuccagacccuuugcuuugcugccaaaguucacgcggccgccaaaguucaaugccuggcucagcacugcuaugcugccugcucuuacugacuggcaugaggaucagcaggggccaguacagccgggaagacaauaacugcacccacuucccagucggccagagccacaugcuccuagagcugcggacugccuucagccaggugaagacuuucuuucaaacaaaggaccagcuggacaacauacugcuaaccgacuccuuaaugcaggacuuuaaggguuacuuggguugccaagccuuaucggaaaugauccaguuuuaccugguagaagugaugccccaggcagagaagcauggcccagaaaucaaggagcauuugaauucccugggugagaagcugaagacccucaggaugcggcugaggcgcugucaucgauuucuccccugugaaaauaagagcaaggcaguggagcaggugaagagugauuuuaauaagcuccaagaccaaggugucuacaaggccaugaaugaauuugacaucuucaucaacugcauagaagcauacaugaugaucaaaaugaaaagcuaaccguaguaagaaaaacuuagggugaaaguucaucgcggccgcagaucuucacgauggccggguuguugagcaccuucgauacauuuagcucuaggaggagcgaaaguauuaauaagucgggaggaggugcuguuauccccggccagaggagcacagucucaguguucguacuaggcccaagugugacugaugaugcagacaaguuauucauugcaacuaccuuccuagcucacucauuggacacagauaagcagcacucucagagagggggguuccucgucucucugcuugccauggcuuacaguaguccagaauuguacuugacaacaaacggaguaaacgccgaugucaaauaugugaucuacaacauagagaaagacccuaagaggacgaagacagacggauucauugugaagacgagagauauggaauaugagaggaccacagaauggcuguuuggaccuauggucaacaagagcccacucuuccagggucaacgggaugcugcagacccugacacacuccuucaaaucuauggguauccugcaugccuaggagcaauaauuguccaagucuggauugugcuggugaaggccaucacaagcagcgccggcuuaaggaaaggguucuucaacagguuagaggcguucagacaagacggcaccgugaaaggugccuuaguuuucacuggggagacaguugaggggauaggcucgguuaugagaucucagcaaagccuuguaucucucaugguugagacccuugugacuaugaauacugcaagaucugaucucaccacauuagagaagaacauccagaucguugggaacuacauccgagaugcagggcuggcuuccuucaugaacacuauuaaauaugggguggaaacaaagauggcagcucuaacguugucaaaccugaggcccgauauuaauaagcuuagaagccucauagacaccuaccugucaaaaggccccagagcucccuuuaucuguauccucaaggacccuguucauggugaauuugcuccaggcaauuauccugcacuauggaguuacgccaugggagucgccgucguacagaacaaggcaaugcagcaguacgucacagggaggacauaccuugauauggaaauguucuuacuaggacaagccguggcaaaggaugcugaaucgaagaucagcagugccuuggaagaugaguuaggagugacggauacagccaaggggaggcucagacaucaucuggcaaacuuguccgguggggauggugcuuaccacaaaccaacaggcgguggugcaauugagguagcucuagacaaugccgacaucgaccuagaaacaaaagcccaugcggaccaggacgcuagggguugggguggagauaguggugaaagaugggcacgucaggugagugguggccacuuugucacacuacauggggcugaacgguuagaggaggaaaccaaugaugaggauguaucagacauagagagaagaauagccaugagacucgcagagagacggcaagaggauucugcaacccauggagaugaaggccgcaauaacggugucgaucaugacgaagaugacgaugccgcagcaguagcugggauaggaggaaucuaggaucauacgaggcuucaagguacuugauccguaguaagaaaaacuuagggugaaaguucauccaccgaucggcucaggcaaggccacacccaaccccaccgaccacacccagcagucgagacagccacggcuucggcuacacuuaccgcauggaucaagaugccuucauucuuaaagaagauucugaaguugagagggaggcgccaggaggacgagagucgcucucggauguuaucggauuccucgaugcuguccugucgagugaaccaacugacaucggaggggacagaagcuggcuccacaacaccaucaacacuccccaaggaccaggcucugcucauagagccaaaagugagggcgaaggagaagucucaacaccgucgacccaagauaaucgaucaggugaggagaguagagucucugggagaacaagcaagccagaggcagaagcacaugcuggaaaccuugauaaacaaaauauacaccgggccuuugggggaagaacugguacaaacucuguaucucaggaucugggcgauggaggagacuccggaauccuugaaaauccuccaaaugagagaggauauccgagaucagguauugaagaugaaaacagagagauggcugcgcacccugauaagaggggagaagaccaagcugaaggacuuccagaagagguacgaggaaguacaucccuaccugaugaaggagaagguggagcaaguaauaauggaagaagcauggagccuggcagcucacauagugcaagaguaacugggguccuggugauuccuagccccgaacuugaagaggcugugcuacggaggaacaaaagaagaccuaccaacaguggguccaaaccucuuacuccagcaaccgugccuggcacccgguccccaccgcugaaucguuacaacagcacagggucaccaccaggaaaacccccaucuacacaggaugagcacaucaacucuggggacacccccgccgucagggucaaagaccggaaaccaccaauagggacccgcucugucucagauuguccagccaacggccgcccaauccacccgggucuagagaccgacucaacaaaaaagggcauaggagagaacacaucaucuaugaaagagauggcuacauuguugacgagucuugguguaauccagucugcucaagaauucgaaucaucccgagacgcgaguuauguguuugcaagacgugcccuaaagucugcaaacuaugcagagaugacauucaauguaugcggccugauccuuucugccgagaaaucuuccgcucguaagguagaugagaacaaacaacugcucaaacagauccaagagagcguggaaucauuccgggauauuuacaagagauucucugaguaucagaaagaacagaacucauugcugauguccaaccuaucuacacuucauaucaucacagauagagguggcaagacugacaacacagacucccuuacaaggucccccuccguuuuugcaaaaucaaaagagaacaagacuaaggcuaccagguuugacccaucuauggagacccuagaagauaugaaguacaaaccggaccuaauccgagaggaugaauuuagagaugagauccgcaacccgguguaccaagagagggacacagaacccagggccucaaacgcaucacgucuccuccccuccaaagagaagcccacaaugcacucucucaggcucgucauagagagcaguccccuaagcagagcugagaaaguagcauaugugaaaucauuauccaagugcaagacagaccaagagguuaaggcagucauggaacucguagaagaggacauagagucacugaccaacuagaucccgggugaggcauccuaccauccucagucauagagagauccaaucuaccaucagcaucagccaguaaagauuaagaaaaacuuagggugaaagaaauuucaccuaacacggcgcaauggcagauaucuauagauucccuaaguucucauaugaggauaacgguacuguggagccccugccucugagaacugguccggauaagaaagccaucccccacaucaggauugucaagguaggagacccuccuaaacauggagugagauaccuagauuuauugcucuuggguuucuuugagacaccgaaacaaacaaccaaucuagggagcguaucugacuugacagagccgaccagcuacucaauaugcggcuccgggucguuacccauagguguggccaaauacuacgggacugaucaggaacucuuaaaggccugcaccgaucucagaauuacggugaggaggacuguucgagcaggagagaugaucguauacaugguggauucgauuggugcuccacuccuaccauggucaggcaggcugagacagggaaugauauuuaaugcaaacaaggucgcacuagcuccccaaugccucccuguggacaaggacauaagacucagagugguguuugucaaugggacaucucuaggggcaaucaccauagccaagaucccaaagacccuugcagaccuugcauugcccaacucuauaucuguuaauuuacuggugacacucaagaccgggaucuccacagaacaaaaggggguacucccaguacuugaugaucaaggggagaaaaagcucaauuuuauggugcaccucggguugaucaggagaaaggucgggaagauauacucuguugaguacugcaagagcaagauugagagaaugcggcugauuuucucacuuggguuaaucggcgguauaagcuuccauguucagguuaaugggacacuaucuaagacauucaugagucagcucgcauggaagagggcagucugcuucccauuaauggaugugaauccccauaugaacauggugauuugggcggcaucuguagaaaucacaggcgucgaugcgguguuccaaccggccaucccucgugauuuccgcuacuacccuaauguuguggcuaagaacaucggaaggaucagaaagcuguaaaugugcacccaucagagaccugcgacaaugccccaagcagacaccaccuggcagucggagccaccgggucacuccuugucuuaaauaagaaaaacuuagggauaaagucccuugugagugcuugguugcaaaacucuccaccugguacaagcacagaucauggauggugauaggggcaaacgugacucguacuggucuacuucuccuagugguagcaccacaaaaccagcaucagguugggagaggucaaguaaagccgacacaugguugcugauucucucauucacccagugggcuuugucaauugccacagugaucaucuguaucauaauuucugcuagacaaggguauaguaugaaagaguacucaaugacuguagaggcauugaacaugagcagcagggaggugaaagagucacuuaccagucuaauaaggcaagagguuauagcaagggcugucaacauucagagcucugugcaaaccggaaucccagucuuguugaacaaaaacagcagggaugucauccagaugauugauaagucgugcagcagacaagagcucacucagcacugugagaguacgaucgcaguccaccaugccgauggaauugccccacuugagccacauaguuucuggagaugcccugucggagaaccguaucuuagcucagauccugaaaucucauugcugccugguccgagcuuguuaucugguucuacaacgaucucuggauguguuaggcucccuucacucucaauuggcgaggcaaucuaugccuauucaucaaaucucauuacacaagguugugcugacauagggaaaucauaucagguccugcagcuaggguacauaucacucaauucagauauguucccugaucuuaaccccguagugucccacacuuaugacaucaacgacaaucggaaaucaugcucugugguggcaaccgggacuagggguuaucagcuuugcuccaugccgacuguagacgaaagaaccgacuacucuagugaugguauugaggaucugguccuugauguccuggaucucaaagggagaacuaagucucaccgguaucgcaacagcgagguagaucuugaucacccguucucugcacuauaccccaguguaggcaacggcauugcaacagaaggcucauugauauuucuuggguaugguggacuaaccaccccucugcagggugauacaaaauguaggacccaaggaugccaacaggugucgcaagacacaugcaaugaggcucugaaaauuacauggcuaggagggaaacagguggucagcgugaucauccaggucaaugacuaucucucagagaggccaaagauaagagucacaaccauuccaaucacucaaaacuaucucggggcggaagguagauuauuaaaauugggugaucggguguacaucuauacaagaucaucaggcuggcacucucaacugcagauaggaguacuugaugucagccacccuuugacuaucaacuggacaccucaugaagccuugucuagaccaggaaauaaagagugcaauugguacaauaaguguccgaaggaaugcauaucaggcguauacacugaugcuuauccauuguccccugaugcagcuaacgucgcuaccgucacgcuauaugccaauacaucgcgugucaacccaacaaucauguauucuaacacuacuaacauuauaaauauguuaaggauaaaggauguucaauuagaggcugcauauaccacgacaucguguaucacgcauuuugguaaaggcuacugcuuucacaucaucgagaucaaucagaagagccugaauaccuuacagccgaugcucuuuaagacuagcaucccuaaauuaugcaaggccgagucuuaaauuuaacugacuagcaggcuugucggccuugcugacacuagagucaucuccgaacauccacaauaucucucagucucuuacgucucucacaguauuaagaaaaacccagggugaaugggaagcuugccauaggucauggaugggcaggaguccucccaaaacccuucugacauacucuauccagaaugccaccugaacucucccauagucagggggaagauagcacaguugcacgucuuguuagaugugaaccagcccuacagacugaaggacgacagcauaauaaauauuacaaagcacaaaauuaggaacggaggauuguccccccgucaaauuaagaucaggucucuggguaaggcucuucaacgcacaauaaaggauuuagaccgauacacguuugaaccguacccaaccuacucucaggaauuacuuaggcuugauauaccagagauaugugacaaaauccgauccgucuucgcggucucggaucggcugaccagggaguuaucuaguggguuccaggaucuuugguugaauaucuucaagcaacuaggcaauauagaaggaagagagggguacgauccguugcaggauaucggcaccaucccggagauaacugauaaguacagcaggaauagaugguauaggccauuccuaacuugguucagcaucaaauaugacaugcgguggaugcagaagaccagaccggggggaccccucgauaccucuaauucacauaaccuccuagaaugcaaaucauacacucuaguaacauacggagaucuugucaugauacugaacaaguugacauugacaggguauauccuaaccccugagcuggucuugauguauugugauguuguagaaggaagguggaauaugucugcugcagggcaucuagauaagaaguccauugggauaacaagcaaaggugaggaauuaugggaacuaguggauucccucuucucaagucuuggagaggaaauauacaaugucaucgcacuauuggagccccuaucacuugcucucauacaacuaaaugauccuguuauaccucuacguggggcauuuaugaggcauguguugacagagcuacagacuguuuuaacaaguagagacguguacacagaugcugaagcagacacuauuguggagucguuacucgccauuuuccauggaaccucuauugaugagaaagcagagaucuuuuccuucuuuaggacauuuggccaccccagcuuagaggcugucacugccgccgacaagguaagggcccauauguaugcacaaaaggcaauaaagcuuaagacccuauacgagugucaugcaguuuuuugcacuaucaucauaaauggguauagagagaggcauggcggacaguggccccccugugacuucccugaucacgugugucuagaacuaaggaacgcucaaggguccaauacggcaaucucuuaugaaugugcuguagacaacuauacaaguuucauaggcuucaaguuucggaaguuuauagaaccacaacuagaugaagaucucacaauauauaugaaagacaaagcacuaucccccaggaaggaggcaugggacucuguauacccggauaguaaucuguacuauaaagccccagagucugaagagacccggcggcuuauugaaguguucauaaaugaugagaauuucaacccagaagaaauuaucaauuauguggagucaggagauugguugaaagacgaggaguucaacaucucguacagucucaaagagaaagagaucaagcaagagggucgucuauucgcaaaaaugacuuauaagaugcgagccguacaggugcuggcagagacacuacuggcuaaaggaauaggagagcuauucagcgaaaaugggaugguuaaaggagagauagaccuacuuaaaagauugacuacucuuucugucucaggcguccccaggacugauucaguguacaauaacucuaaaucaucagagaagagaaacgaaggcauggaaaauaagaacucugggggguacugggacgaaaagaagagguccagacaugaauucaaggcaacagauucaucaacagacggcuaugaaacguuaaguugcuuccucacaacagaccucaagaaauacugcuuaaacuggagauuugagaguacugcauuguuuggucagagaugcaacgagauauuuggcuucaagaccuucuuuaacuggaugcauccaguccuugaaagguguacaauauauguuggagauccuuacuguccagucgccgaccggaugcaucgacaacuccaggaucaugcagacucuggcauuuucauacauaauccuagggggggcauagaagguuacugccagaagcuguggaccuuaaucucaaucagugcaauccaccuagcagcugugagagugggugucagggucucugcaaugguucagggugacaaucaagcuauagccgugacaucaagaguaccuguagcucagacuuacaagcagaagaaaaaucaugucuaugaggagaucaccaaauauuucggugcucuaagacacgucauguuugauguagggcacgagcuaaaauugaacgagaccaucauuaguagcaagauguuugucuauaguaaaaggauauacuaugaugggaagauuuuaccacagugccugaaagccuugaccaaguguguauucugguccgagacacugguagaugaaaacagaucugcuuguucgaacaucucaacauccauagcaaaagcuaucgaaaauggguauucuccuauacuaggcuacugcauugcguuguauaagaccugucagcaggugugcauaucacuagggaugacuauaaauccaacuaucagcccgaccguaagagaucaauacuuuaaggguaagaauuggcugagaugugcaguguugauuccagcaaauguuggaggauucaacuacaugucuacaucuagaugcuuuguuagaaauauuggagaccccgcaguagcagcccuagcugaucucaaaagauucaucagagcggaucuguuagacaagcagguauuauacagggucaugaaucaagaacccggugacucuaguuuucuagauugggcuucagacccuuauucguguaaccucccgcauucucagaguauaacuacgauuauaaagaauaucacugcuagaucugugcugcaggaauccccgaauccucuacugucuggucucuucaccgagacuaguggagaagaggaucucaaccuggccucguuccuuauggaccggaaagucauccugccgagaguggcucaugagauccuggguaauuccuuaacuggaguuagggaggcgauugcagggaugcuugauacgaccaagucucuagugagagccagcguuaggaaaggaggauuaucauaugggauauugaggaggcuugucaauuaugaucuauugcaguacgagacacugacuagaacucucaggaaaccggugaaagacaacaucgaauaugaguauauguguucaguugagcuagcugucggucuaaggcagaaaauguggauccaccugacuuacgggagacccauacaugggcuagaaacaccagacccuuuagagcucuugaggggaauauuuaucgaagguucagaggugugcaagcuuugcaggucugaaggagcagaccccaucuauacaugguucuaucuuccugacaauauagaccuggacacgcuuacaaacggauguccggcuauaagaauccccuauuuuggaucagccacugaugaaaggucggaagcccaacucggguauguaagaaaucuaagcaaacccgcaaaggcggccauccggauagcuaugguguauacgugggccuacgggacugaugagauaucguggauggaagccgcucuuauagcccaaacaagagcuaaucugagcuuagagaaucuaaagcugcugacuccuguuucaaccuccacuaaucuaucucauagguugaaagauacggcaacccagaugaaguucucuagugcaacacuaguccgugcaagucgguucauaacaauaucaaaugauaacauggcacucaaagaagcaggggagucgaaggauacuaaucucguguaucagcagauuaugcuaacugggcuaagcuuguucgaguucaauaugagauauaagaaagguuccuuagggaagccacugauauugcacuuacaucuuaauaacgggugcuguauaauggaguccccacaggaggcgaauauccccccaagguccacauuagauuuagagauuacacaagagaacaauaaauugaucuaugauccugauccacucaaggauguggaccuugagcuauuuagcaaggucagagauguuguacacacaguugacaugacuuauuggucagaugaugaaguuaucagagcaaccaguaucuguacugcaaugacgauagcugauacaaugucucaauuagauagagacaacuuaaaagagaugaucgcacuaguaaaugacgaugaugucaacagcuugauuacugaguuuauggugauugauguuccuuuauuuugcucaacguucggggguauucuagucaaucaguuugcauacucacucuacggcuuaaacaucagaggaagggaagaaauauggggacauguaguccggauucuuaaagauaccucccacgcaguuuuaaaagucuuaucuaaugcucuaucucaucccaaaaucuucaaacgauucuggaaugcaggugucguggaaccuguguaugggccuaaccucucaaaucaggauaagauacucuuggcccucucugucugugaauauucuguggaucuauucaugcacgauuggcaaggggguguaccgcuugagaucuuuaucugugacaaugacccagauguggccgacaugaggagguccucuuucuuggcaagacaucuugcauaccuaugcagcuuggcagagauaucuagggaugggccaagauuagaaucaaugaacucucuagagaggcucgagucacuaaagaguuaccuggaacucacauuucuugaugacccgguacugagguacagucaguugacuggccuagucaucaaaguauucccaucuacuuugaccuauauccggaagucaucuauaaaaguguuaaggacaagagguauaggagucccugaagucuuagaagauugggaucccgaggcagauaaugcacuguuagaugguaucgcggcagaaauacaacagaauauuccuuugggacaucagacuagagccccuuuuuggggguugagaguauccaagucacagguacugcgucuccggggguacaaggagaucacaagaggugagauaggcagaucagguguuggucugacguuaccauucgauggaagauaucuaucucaccagcugaggcucuuuggcaucaacaguacuagcugcuugaaagcacuugaacuuaccuaccuauugagccccuuaguugacaaggauaaagauaggcuauauuuaggggaaggagcuggggccaugcuuuccuguuaugacgcuacucuuggcccaugcaucaacuauuauaacucagggguauacucuugugaugucaaugggcagagagaguuaaauauauauccugcugagguggcacuagugggaaagaaauuaaacaauguuacuagucugggucaaagaguuaaaguguuauucaacgggaauccuggcucgacauggauugggaaugaugagugugaggcuuugauuuggaaugaauuacagaauagcucgauaggccuaguccacugugacauggagggaggagaucauaaggaugaucaaguuguacugcaugagcauuacaguguaauccggaucgcguaucuggugggggaucgagacguugugcuuauaagcaagauugcucccaggcugggcacggauuggaccaggcagcucagccuauaucugagauacugggacgagguuaaccuaauagugcuuaaaacaucuaacccugcuuccacagagauguaucuccuaucgaggcaccccaaaucugacauuauagaggacagcaagacaguguuagcuagucuccucccuuugucaaaagaagauagcaucaagauagaaaaguggaucuuaauagagaaggcaaaggcucacgaauggguuacucgggaauugagagaaggaagcucuucaucagggaugcuuagaccuuaccaucaagcacugcagacguuuggcuuugaaccaaacuuguauaaauugagcagagauuucuuguccaccaugaacauagcugauacacacaacugcaugauagcuuucaacaggguuuugaaggauacaaucuucgaaugggcuagaauaacugagucagauaaaaggcuuaaacuaacugguaaguaugaccuguauccugugagagauucaggcaaguugaagacaauuucuagaagacuugugcuaucuuggauaucuuuaucuauguccacaagauugguaacugggucauucccugaccagaaguuugaagcaagacuucaauugggaauaguuucauuaucaucccgugaaaucaggaaccugaggguuaucacaaaaacuuuauuagacagguuugaggauauuauacauaguauaacguauagauuccucaccaaagaaauaaagauuuugaugaagauuuuaggggcagucaagauguucggggccaggcaaaaugaauacacgaccgugauugaugauggaucacuaggugauaucgagccauaugacagcucguaauaauuagucccuaucgugcagaacgaucgaagcuccgcgguaccuggaagucuuggacuuguccauaugacaauaguaagaaaaacuuacaagaagacaagaaaauuuaaaaggauacauaucucuuaaacucuugucuggu

SEQ ID NO: 18 - RNA, 14172 nt, Artificial
  SeV18+mIL-10/TS-dF (genomic RNA)
accagacaagaguuuaagagauauguauccuuuuaaauuuucuugucuucuuguaaguuuuucuuacuauugucauauggacaaguccaagacuuccagguaccgcggagcuucgaucguucugcacgauagggacuaauuauuacgagcugucauauggcucgauaucaccuagugauccaucaucaaucacggucguguauucauuuugccuggccccgaacaucuugacugccccuaaaaucuucaucaaaaucuuuauuucuuuggugaggaaucuauacguuauacuauguauaauauccucaaaccugucuaauaaaguuuuugugauaacccucagguuccugauuucacgggaugauaaugaaacuauucccaauugaagucuugcuucaaacuucuggucagggaaugacccaguuaccaaucuuguggacauagauaaagauauccaagauagcacaagucuucuagaaauugucuucaacuugccugaaucucucacaggauacaggucauacuuaccaguuaguuuaagccuuuuaucugacucaguuauucuagcccauucgaagauuguauccuucaaaacccuguugaaagcuaucaugcaguuguguguaucagcuauguucaugguggacaagaaaucucugcucaauuuauacaaguuugguucaaagccaaacgucugcagugcuugaugguaaggucuaagcaucccugaugaagagcuuccuucucucaauucccgaguaacccauucgugagccuuugccuucucuauuaagauccacuuuucuaucuugaugcuaucuucuuuugacaaagggaggagacuagcuaacacugucuugcuguccucuauaaugucagauuuggggugccucgauaggagauacaucucuguggaagcaggguuagauguuuuaagcacuauuagguuaaccucgucccaguaucucagauauaggcugagcugccugguccaauccgugcccagccugggagcaaucuugcuuauaagcacaacgucucgaucccccaccagauacgcgauccggauuacacuguaaugcucaugcaguacaacuugaucauccuuaugaucuccucccuccaugucacaguggacuaggccuaucgagcuauucuguaauucauuccaaaucaaagccucacacucaucauucccaauccaugucgagccaggauucccguugaauaacacuuuaacucuuugacccagacuaguaacauuguuuaauuucuuucccacuagugccaccucagcaggauauauauuuaacucucucugcccauugacaucacaagaguauaccccugaguuauaauaguugaugcaugggccaagaguagcgucauaacaggaaagcauggccccagcuccuuccccuaaauauagccuaucuuuaucuucgucaacuaaggggcucaauagguagguaaguucaagugcuuucaagcagcuaguacuguugaugccaaagagccucagcuggugagauagauaucuuccaucgaaugguaacgucagaccaacaccugaucugccuaucucaccucuugugaucuccuuguacccccggagacgcaguaccugugacuuggauacucucaacccccaaaaaggggcucuagucugaugucccaaaggaauauucuguuguauuucugccgcgauaccaucuaacagugcauuaucugccucgggaucccaaucuucuaagacuucagggacuccuauaccucuuguccuuaacacuuuuauagaugacuuccggauauaggucaaaguagaugggaauacuuugaugacuaggccagucaacugacuguaccucaguaccgggucaucaagaaaugugaguuccagguaacucuuuagugacucgagccucucuagagaguucauugauucuaaucuuggcccaucccuagauaucucugccaagcugcauagguaugcaagaugucuugccaagaaagaggaccuccucaugucggccacaucugggucauugucacagauaaagaucucaagcgguacacccccuugccaaucgugcaugaauagauccacagaauauucacagacagagagggccaagaguaucuuauccugauuugagagguuaggcccauacacagguuccacgacaccugcauuccagaaucguuugaagauuuugggaugagauagagcauuagauaagacuuuuaaaacugcgugggagguaucuuuaagaauccggacuacauguccccauauuucuucccuuccucugauguuuaagccguagagugaguaugcaaacugauugacuagaauacccccgaacguugagcaaaauaaaggaacaucaaucaccauaaacucaguaaucaagcuguugacaucaucgucauuuacuagugcgaucaucucuuuuaaguugucucuaucuaauugagacauuguaucagcuaucgucauugcaguacagauacugguugcucugauaacuucaucaucugaccaauaagucaugucaacuguguguacaacaucucugaccuugcuaaauagcucaagguccacauccuugaguggaucaggaucauagaucaauuuauuguucucuuguguaaucucuaaaucuaauguggaccuuggggggauauucgccuccuguggggacuccauuauacagcacccguuauuaagauguaagugcaauaucaguggcuucccuaaggaaccuuucuuauaucucauauugaacucgaacaagcuuagcccaguuagcauaaucugcugauacacgagauuaguauccuucgacuccccugcuucuuugagugccauguuaucauuugauauuguuaugaaccgacuugcacggacuaguguugcacuagagaacuucaucuggguugccguaucuuucaaccuaugagauagauuaguggagguugaaacaggagucagcagcuuuagauucucuaagcucagauuagcucuuguuugggcuauaagagcggcuuccauccacgauaucucaucagucccguaggcccacguauacaccauagcuauccggauggccgccuuugcggguuugcuuagauuucuuacauacccgaguugggcuuccgaccuuucaucaguggcugauccaaaauaggggauucuuauagccggacauccguuuguaagcguguccaggucuauagagucaggaagauagaaccauguauagauggggucugcuccuucagaccugcaaagcuugcacaccucugaaccuucgauaaauauuccccucaagagcucuaaagggucugguguuucuagcccauguaugggucucccguaagucagguggauccacauuuucugccuuagaccgacagcuagcucaacugaacacauauacucauauucgauguugucuuucaccgguuuccugagaguucuagucagugucucguacugcaauagaucauaauugacaagccuccucaauaucccauaugauaauccuccuuuccuaacgcuggcucucacuagagacuuggucguaucaagcaucccugcaaucgccucccuaacuccaguuaaggaauuacccaggaucucaugagccacucucggcaggaugacuuuccgguccauaaggaacgaggccagguugagauccucuucuccacuagucucggugaagagaccagacaguagaggauucggggauuccugcagcacagaucuagcagugauauucuuuauaaucguaguuauacucugagaaugcgggagguuacacgaauaagggucugaagcccaaucuagaaaacuagagucaccggguucuugauucaugacccuguauaauaccugcuugucuaacagauccgcucugaugaaucuuuugagaucagcuagggcugcuacugcggggucuccaauauuucuaacaaagcaucuagauguagacauguaguugaauccuccaacauuugcuggaaucaacacugcacaucucagccaauucuuacccuuaaaguauugaucucuuacggucgggcugauaguuggauuuauagucaucccuagugauaugcacaccugcugacaggucuuauacaacgcaaugcaguagccuaguauaggagaauacccauuuucgauagcuuuugcuauggauguugagauguucgaacaagcagaucuguuuucaucuaccagugucucggaccagaauacacacuuggucaaggcuuucaggcacugugguaaaaucuucccaucauaguauauccuuuuacuauagacaaacaucuugcuacuaaugauggucucguucaauuuuagcucgugcccuacaucaaacaugacgugucuuagagcaccgaaauauuuggugaucuccucauagacaugauuuuucuucugcuuguaagucugagcuacagguacucuugaugucacggcuauagcuugauugucacccugaaccauugcagagacccugacacccacucucacagcugcuagguggauugcacugauugagauuaagguccacagcuucuggcaguaaccuucuaugcccccccuaggauuauguaugaaaaugccagagucugcaugauccuggaguugucgaugcauccggucggcgacuggacaguaaggaucuccaacauauauuguacaccuuucaaggacuggaugcauccaguuaaagaaggucuugaagccaaauaucucguugcaucucugaccaaacaaugcaguacucucaaaucuccaguuuaagcaguauuucuugaggucuguugugaggaagcaacuuaacguuucauagccgucuguugaugaaucuguugccuugaauucaugucuggaccucuucuuuucgucccaguaccccccagaguucuuauuuuccaugccuucguuucucuucucugaugauuuagaguuauuguacacugaaucaguccuggggacgccugagacagaaagaguagucaaucuuuuaaguaggucuaucucuccuuuaaccaucccauuuucgcugaauagcucuccuauuccuuuagccaguagugucucugccagcaccuguacggcucgcaucuuauaagucauuuuugcgaauagacgacccucuugcuugaucucuuucucuuugagacuguacgagauguugaacuccucgucuuucaaccaaucuccugacuccacauaauugauaauuucuucuggguugaaauucucaucauuuaugaacacuucaauaagccgccgggucucuucagacucuggggcuuuauaguacagauuacuauccggguauacagagucccaugccuccuuccugggggauagugcuuugucuuucauauauauugugagaucuucaucuaguugugguucuauaaacuuccgaaacuugaagccuaugaaacuuguauaguugucuacagcacauucauaagagauugccguauuggacccuugagcguuccuuaguucuagacacacgugaucagggaagucacaggggggccacuguccgccaugccucucucuauacccauuuaugaugauagugcaaaaaacugcaugacacucguauagggucuuaagcuuuauugccuuuugugcauacauaugggcccuuaccuugucggcggcagugacagccucuaagcugggguggccaaauguccuaaagaaggaaaagaucucugcuuucucaucaauagagguuccauggaaaauggcgaguaacgacuccacaauagugucugcuucagcaucuguguacacgucucuacuuguuaaaacagucuguagcucugucaacacaugccucauaaaugccccacguagagguauaacaggaucauuuaguuguaugagagcaagugauaggggcuccaauagugcgaugacauuguauauuuccucuccaagacuugagaagagggaauccacuaguucccauaauuccucaccuuugcuuguuaucccaauggacuucuuaucuagaugcccugcagcagacauauuccaccuuccuucuacaacaucacaauacaucaagaccagcucagggguuaggauauacccugucaaugucaacuuguucaguaucaugacaagaucuccguauguuacuagaguguaugauuugcauucuaggagguuaugugaauuagagguaucgagggguccccccggucuggucuucugcauccaccgcaugucauauuugaugcugaaccaaguuaggaauggccuauaccaucuauuccugcuguacuuaucaguuaucuccgggauggugccgauauccugcaacggaucguaccccucucuuccuucuauauugccuaguugcuugaagauauucaaccaaagauccuggaacccacuagauaacucccuggucagccgauccgagaccgcgaagacggaucggauuuugucacauaucucugguauaucaagccuaaguaauuccugagaguagguuggguacgguucaaacguguaucggucuaaauccuuuauugugcguugaagagccuuacccagagaccugaucuuaauuugacggggggacaauccuccguuccuaauuuugugcuuuguaauauuuauuaugcugucguccuucagucuguagggcugguucacaucuaacaagacgugcaacugugcuaucuucccccugacuaugggagaguucagguggcauucuggauagaguaugucagaaggguuuugggaggacuccugcccauccaugaccuauggcaagcuucccauucacccuggguuuuucuuaauacugugagagacguaagagacugagagauauuguggauguucggagaugacucuagugucagcaaggccgacaagccugcuagucaguuaaauuuaagacucggccuugcauaauuuagggaugcuagucuuaaagagcaucggcuguaagguauucaggcucuucugauugaucucgaugaugugaaagcaguagccuuuaccaaaaugcgugauacacgaugucgugguauaugcagccucuaauugaacauccuuuauccuuaacauauuuauaauguuaguaguguuagaauacaugauuguuggguugacacgcgauguauuggcauauagcgugacgguagcgacguuagcugcaucaggggacaauggauaagcaucaguguauacgccugauaugcauuccuucggacacuuauuguaccaauugcacucuucauuuccuggucuagacaaggcuucaugagguguccaguugauagucaaaggguggcugacaucaaguacuccuaucugcaguugagagugccagccugaugaucuuguauagauguacacccgaucacccaauuuuaauaaucuaccuuccgccccgagauaguuuugagugauuggaaugguugugacucuuaucuuuggccucucugagagauagucauugaccuggaugaucacgcugaccaccuguuucccuccuagccauguaauuuucagagccucauugcaugugucuugcgacaccuguuggcauccuuggguccuacauuuuguaucacccugcagaggggugguuaguccaccauacccaagaaauaucaaugagccuucuguugcaaugccguugccuacacugggguauagugcagagaacgggugaucaagaucuaccucgcuguugcgauaccggugagacuuaguucucccuuugagauccaggacaucaaggaccagauccucaauaccaucacuagaguagucgguucuuucgucuacagucggcauggagcaaagcugauaaccccuaguccggguugucaccacagagcaugauuuccgauugucguugaugucauaagugugggacacuacgggguuaagaucagggaacauaucugaauugagugauauguacccuagcugcaggaccugauaugauuucccuaugucagcacaaccuuguguaaugagauuugaugaauaggcauagauugccucgccaauugagagugaagggagccuaacacauccagagaucguuguagaaccagauaacaagcucggaccaggcagcaaugagauuucaggaucugagcuaagauacgguucuccgacagggcaucuccagaaacuauguggcucaaguggggcaauuccaucggcaugguggacugcgaucguacucucacagugcugagugagcucuugucugcugcacgacuuaucaaucaucuggaugacaucccugcuguuuuuguucaacaagacugggauuccgguuugcacagagcucugaauguugacagcccuugcuauaaccucuugccuuauuagacugguaagugacucuuucaccucccugcugcucauguucaaugccucuacagucauugaguacucuuucauacuauacccuugucuagcagaaauuaugauacagaugaucacuguggcaauugacaaagcccacugggugaaugagagaaucagcaaccaugugucggcuuuacuugaccucucccaaccugaugcugguuuuguggugcuaccacuaggagaaguagaccaguacgagucacguuugccccuaucaccauccaugaucugugcuuguuugaggugaaagcuuaauuaaccaagcacucacaagggacuuuaucccuaaguuuuucuuauuuaagacaaggagugacccgguggcuccgacugccagguggugucugcuuggggcauugucgcaggucucugaugggugcacauuuacagcuuucugauccuuccgauguucuuagccacaacauuaggguaguagcggaaaucacgagggauggccgguuggaacaccgcaucgacgccugugauuucuacagaugccgcccaaaucaccauguucauauggggauucacauccauuaaugggaagcagacugcccucuuccaugcgagcugacucaugaaugucuuagauagugucccauuaaccugaacauggaagcuuauaccgccgauuaacccaagugagaaaaucagccgcauucucucaaucuugcucuugcaguacucaacagaguauaucuucccgaccuuucuccugaucaacccgaggugcaccauaaaauugagcuuuuucuccccuugaucaucaaguacugggaguacccccuuuuguucuguggagaucccggucuugagugucaccaguaaauuaacagauauagaguugggcaaugcaaggucugcaagggucuuugggaucuuggauauggugauugccccuagagaugucccauugacaaacaccacucugagucuuauguccuuguccacagggaggcauuggggagcuagugcgaccuuguuugcauuaaauaucauucccugucucagccugccugaccaugguaggaguggagcaccaaucgaauccaccauguauacgaucaucucuccugcucgaacagcccuccucaccguaauucugagaucggugcaggccuuuaagaguuccugaucagucccguaguauuuggccacaccuauggguaacgacccggagccgcauauugaguagcuggucggcucugucaagucagauacgcucucuagauugguuguuuguuucggugucucaaagaaacccaagagcaauaaaucuagguaucucacuccauguuuaggagggucuccuaccuugacaauccugaugugggggauggcuuucuuauccggaccaguucucagaggcaggggcuccacaguaccguuauccucauaugagaacuuagggaaucuauagauaucugccauugcgccguguuaggugaaauuucuuucacccuaaguuuuucuuaaucuuuacuggcugaugcugaugguagauuggaucucucuaugacugaggaugguaggaugccucacccgggaucuaguuggucagugacucuauguccucuucuacgaguuccaugacugccuuaaccucuuggucugucuugcacuuggauaaugauuucacauaugcuacuuucucagcucugcuuaggggacugcucucuaugacgagccugagagagugcauugugggcuucucuuuggagggaaagagacgugaugcguuugaggcccuggguucugugucccucucuugguacaccggguugcggaucucaucucuaaauucauccucucggauuagguccgguuuguacuucauaucuucuagggucuccauagaugggucaaaccugguagccuuagucuuguucucuuuugauuuugcaaaaacggagggggaccuuguaagggagucuguguugucagucuugccaccucuaucugugaugauaugaaguguagauagguuggacaucagcaaugaguucuguucuuucugauacucagagaaucucuuguaaauaucccggaaugauuccacgcucucuuggaucuguuugagcaguuguuuguucucaucuaccuuacgagcggaagauuucucggcagaaaggaucaggccgcauacauugaaugucaucucugcauaguuugcagacuuuagggcacgucuugcaaacacauaacucgcgucucgggaugauucgaauucuugagcagacuggauuacaccaagacucgucaacaauguagccaucucuuucauagaugauguguucucuccuaugcccuuuuuuguugagucggucucuagacccggguggauugggcggccguuggcuggacaaucugagacagagcgggucccuauuggugguuuccggucuuugacccugacggcggggguguccccagaguugaugugcucauccuguguagauggggguuuuccugguggugacccugugcuguuguaacgauucagcgguggggaccgggugccaggcacgguugcuggaguaagagguuuggacccacuguugguaggucuucuuuuguuccuccguagcacagccucuucaaguucggggcuaggaaucaccaggaccccaguuacucuugcacuaugugagcugccaggcuccaugcuucuuccauuauuacuugcuccaccuucuccuucaucagguagggauguacuuccucguaccucuucuggaaguccuucagcuuggucuucuccccucuuaucagggugcgcagccaucucucuguuuucaucuucaauaccugaucucggauauccucucucauuuggaggauuuucaaggauuccggagucuccuccaucgcccagauccugagauacagaguuuguaccaguucuucccccaaaggcccgguguauauuuuguuuaucaagguuuccagcaugugcuucugccucuggcuugcuuguucucccagagacucuacucuccucaccugaucgauuaucuugggucgacgguguugagacuucuccuucgcccucacuuuuggcucuaugagcagagccugguccuuggggaguguugaugguguuguggagccagcuucuguccccuccgaugucaguugguucacucgacaggacagcaucgaggaauccgauaacauccgagagcgacucucguccuccuggcgccucccucucaacuucagaaucuucuuuaagaaugaaggcaucuugauccaugcgguaaguguagccgaagccguggcugucucgacugcuggguguggucggugggguuggguguggccuugccugagccgaucgguggaugaacuuucacccuaaguuuuucuuacuacggaucaaguaccuugaagccucguaugauccuagauuccuccuaucccagcuacugcugcggcaucgucaucuucgucaugaucgacaccguuauugcggccuucaucuccauggguugcagaauccucuugccgucucucugcgagucucauggcuauucuucucucuaugucugauacauccucaucauugguuuccuccucuaaccguucagccccauguagugugacaaaguggccaccacucaccugacgugcccaucuuucaccacuaucuccaccccaaccccuagcguccugguccgcaugggcuuuuguuucuaggucgaugucggcauugucuagagcuaccucaauugcaccaccgccuguugguuugugguaagcaccauccccaccggacaaguuugccagaugaugucugagccuccccuuggcuguauccgucacuccuaacucaucuuccaaggcacugcugaucuucgauucagcauccuuugccacggcuuguccuaguaagaacauuuccauaucaagguauguccucccugugacguacugcugcauugccuuguucuguacgacggcgacucccauggcguaacuccauagugcaggauaauugccuggagcaaauucaccaugaacaggguccuugaggauacagauaaagggagcucuggggccuuuugacagguaggugucuaugaggcuucuaagcuuauuaauaucgggccucagguuugacaacguuagagcugccaucuuuguuuccaccccauauuuaauaguguucaugaaggaagccagcccugcaucucggauguaguucccaacgaucuggauguucuucucuaauguggugagaucagaucuugcaguauucauagucacaagggucucaaccaugagagauacaaggcuuugcugagaucucauaaccgagccuauccccucaacugucuccccagugaaaacuaaggcaccuuucacggugccgucuugucugaacgccucuaaccuguugaagaacccuuuccuuaagccggcgcugcuugugauggccuucaccagcacaauccagacuuggacaauuauugcuccuaggcaugcaggauacccauagauuugaaggagugugucagggucugcagcaucccguugacccuggaagagugggcucuuguugaccauagguccaaacagccauucugugguccucucauauuccauaucucucgucuucacaaugaauccgucugucuucguccucuuagggucuuucucuauguuguagaucacauauuugacaucggcguuuacuccguuuguugucaaguacaauucuggacuacuguaagccauggcaagcagagagacgaggaaccccccucucugagagugcugcuuaucuguguccaaugagugagcuaggaagguaguugcaaugaauaacuugucugcaucaucagucacacuugggccuaguacgaacacugagacugugcuccucuggccggggauaacagcaccuccucccgacuuauuaauacuuucgcuccuccuagagcuaaauguaucgaaggugcucaacaacccggccaucgugaagaucugcggccgcgaugaacuuucacccuaaguuuuucuuacuacgguuagcuuuucauuuugaucaucauguaugcuucuaugcaguugaugaagaugucaaauucauucauggccuuguagacaccuuggucuuggagcuuauuaaaaucacucuucaccugcuccacugccuugcucuuauuuucacaggggagaaaucgaugacagcgccucagccgcauccugagggucuucagcuucucacccagggaauucaaaugcuccuugauuucugggccaugcuucucugccuggggcaucacuucuaccagguaaaacuggaucauuuccgauaaggcuuggcaacccaaguaacccuuaaaguccugcauuaaggagucgguuagcaguauguuguccagcugguccuuuguuugaaagaaagucuucaccuggcugaaggcaguccgcagcucuaggagcauguggcucuggccgacugggaagugggugcaguuauugucuucccggcuguacuggccccugcugauccucaugccagucaguaagagcaggcagcauagcagugcugagccaggcauugaacuuuggcggccgcgugaacuuuggcagcaaagcaaagggucuggaaccugcuccucaggguggauacuuugacccuaaaauccuguauaacuucauuacauaucccauacauguuuuuucucuuguuuggu

SEQ ID NO: 19 - RNA, 14172 nt, Artificial
  SeV18+mIL-10/TS-dF (antigenomic RNA)
accaaacaagagaaaaaacauguaugggauauguaaugaaguuauacaggauuuuagggucaaaguauccacccugaggagcagguuccagacccuuugcuuugcugccaaaguucacgcggccgccaaaguucaaugccuggcucagcacugcuaugcugccugcucuuacugacuggcaugaggaucagcaggggccaguacagccgggaagacaauaacugcacccacuucccagucggccagagccacaugcuccuagagcugcggacugccuucagccaggugaagacuuucuuucaaacaaaggaccagcuggacaacauacugcuaaccgacuccuuaaugcaggacuuuaaggguuacuuggguugccaagccuuaucggaaaugauccaguuuuaccugguagaagugaugccccaggcagagaagcauggcccagaaaucaaggagcauuugaauucccugggugagaagcugaagacccucaggaugcggcugaggcgcugucaucgauuucuccccugugaaaauaagagcaaggcaguggagcaggugaagagugauuuuaauaagcuccaagaccaaggugucuacaaggccaugaaugaauuugacaucuucaucaacugcauagaagcauacaugaugaucaaaaugaaaagcuaaccguaguaagaaaaacuuagggugaaaguucaucgcggccgcagaucuucacgauggccggguuguugagcaccuucgauacauuuagcucuaggaggagcgaaaguauuaauaagucgggaggaggugcuguuauccccggccagaggagcacagucucaguguucguacuaggcccaagugugacugaugaugcagacaaguuauucauugcaacuaccuuccuagcucacucauuggacacagauaagcagcacucucagagagggggguuccucgucucucugcuugccauggcuuacaguaguccagaauuguacuugacaacaaacggaguaaacgccgaugucaaauaugugaucuacaacauagagaaagacccuaagaggacgaagacagacggauucauugugaagacgagagauauggaauaugagaggaccacagaauggcuguuuggaccuauggucaacaagagcccacucuuccagggucaacgggaugcugcagacccugacacacuccuucaaaucuauggguauccugcaugccuaggagcaauaauuguccaagucuggauugugcuggugaaggccaucacaagcagcgccggcuuaaggaaaggguucuucaacagguuagaggcguucagacaagacggcaccgugaaaggugccuuaguuuucacuggggagacaguugaggggauaggcucgguuaugagaucucagcaaagccuuguaucucucaugguugagacccuugugacuaugaauacugcaagaucugaucucaccacauuagagaagaacauccagaucguugggaacuacauccgagaugcagggcuggcuuccuucaugaacacuauuaaauaugggguggaaacaaagauggcagcucuaacguugucaaaccugaggcccgauauuaauaagcuuagaagccucauagacaccuaccugucaaaaggccccagagcucccuuuaucuguauccucaaggacccuguucauggugaauuugcuccaggcaauuauccugcacuauggaguuacgccaugggagucgccgucguacagaacaaggcaaugcagcaguacgucacagggaggacauaccuugauauggaaauguucuuacuaggacaagccguggcaaaggaugcugaaucgaagaucagcagugccuuggaagaugaguuaggagugacggauacagccaaggggaggcucagacaucaucuggcaaacuuguccgguggggauggugcuuaccacaaaccaacaggcgguggugcaauugagguagcucuagacaaugccgacaucgaccuagaaacaaaagcccaugcggaccaggacgcuagggguugggguggagauaguggugaaagaugggcacgucaggugagugguggccacuuugucacacuacauggggcugaacgguuagaggaggaaaccaaugaugaggauguaucagacauagagagaagaauagccaugagacucgcagagagacggcaagaggauucugcaacccauggagaugaaggccgcaauaacggugucgaucaugacgaagaugacgaugccgcagcaguagcugggauaggaggaaucuaggaucauacgaggcuucaagguacuugauccguaguaagaaaaacuuagggugaaaguucauccaccgaucggcucaggcaaggccacacccaaccccaccgaccacacccagcagucgagacagccacggcuucggcuacacuuaccgcauggaucaagaugccuucauucuuaaagaagauucugaaguugagagggaggcgccaggaggacgagagucgcucucggauguuaucggauuccucgaugcuguccugucgagugaaccaacugacaucggaggggacagaagcuggcuccacaacaccaucaacacuccccaaggaccaggcucugcucauagagccaaaagugagggcgaaggagaagucucaacaccgucgacccaagauaaucgaucaggugaggagaguagagucucugggagaacaagcaagccagaggcagaagcacaugcuggaaaccuugauaaacaaaauauacaccgggccuuugggggaagaacugguacaaacucuguaucucaggaucugggcgauggaggagacuccggaauccuugaaaauccuccaaaugagagaggauauccgagaucagguauugaagaugaaaacagagagauggcugcgcacccugauaagaggggagaagaccaagcugaaggacuuccagaagagguacgaggaaguacaucccuaccugaugaaggagaagguggagcaaguaauaauggaagaagcauggagccuggcagcucacauagugcaagaguaacugggguccuggugauuccuagccccgaacuugaagaggcugugcuacggaggaacaaaagaagaccuaccaacaguggguccaaaccucuuacuccagcaaccgugccuggcacccgguccccaccgcugaaucguuacaacagcacagggucaccaccaggaaaacccccaucuacacaggaugagcacaucaacucuggggacacccccgccgucagggucaaagaccggaaaccaccaauagggacccgcucugucucagauuguccagccaacggccgcccaauccacccgggucuagagaccgacucaacaaaaaagggcauaggagagaacacaucaucuaugaaagagauggcuacauuguugacgagucuugguguaauccagucugcucaagaauucgaaucaucccgagacgcgaguuauguguuugcaagacgugcccuaaagucugcaaacuaugcagagaugacauucaauguaugcggccugauccuuucugccgagaaaucuuccgcucguaagguagaugagaacaaacaacugcucaaacagauccaagagagcguggaaucauuccgggauauuuacaagagauucucugaguaucagaaagaacagaacucauugcugauguccaaccuaucuacacuucauaucaucacagauagagguggcaagacugacaacacagacucccuuacaaggucccccuccguuuuugcaaaaucaaaagagaacaagacuaaggcuaccagguuugacccaucuauggagacccuagaagauaugaaguacaaaccggaccuaauccgagaggaugaauuuagagaugagauccgcaacccgguguaccaagagagggacacagaacccagggccucaaacgcaucacgucucuuucccuccaaagagaagcccacaaugcacucucucaggcucgucauagagagcaguccccuaagcagagcugagaaaguagcauaugugaaaucauuauccaagugcaagacagaccaagagguuaaggcagucauggaacucguagaagaggacauagagucacugaccaacuagaucccgggugaggcauccuaccauccucagucauagagagauccaaucuaccaucagcaucagccaguaaagauuaagaaaaacuuagggugaaagaaauuucaccuaacacggcgcaauggcagauaucuauagauucccuaaguucucauaugaggauaacgguacuguggagccccugccucugagaacugguccggauaagaaagccaucccccacaucaggauugucaagguaggagacccuccuaaacauggagugagauaccuagauuuauugcucuuggguuucuuugagacaccgaaacaaacaaccaaucuagagagcguaucugacuugacagagccgaccagcuacucaauaugcggcuccgggucguuacccauagguguggccaaauacuacgggacugaucaggaacucuuaaaggccugcaccgaucucagaauuacggugaggagggcuguucgagcaggagagaugaucguauacaugguggauucgauuggugcuccacuccuaccauggucaggcaggcugagacagggaaugauauuuaaugcaaacaaggucgcacuagcuccccaaugccucccuguggacaaggacauaagacucagagugguguuugucaaugggacaucucuaggggcaaucaccauauccaagaucccaaagacccuugcagaccuugcauugcccaacucuauaucuguuaauuuacuggugacacucaagaccgggaucuccacagaacaaaaggggguacucccaguacuugaugaucaaggggagaaaaagcucaauuuuauggugcaccucggguugaucaggagaaaggucgggaagauauacucuguugaguacugcaagagcaagauugagagaaugcggcugauuuucucacuuggguuaaucggcgguauaagcuuccauguucagguuaaugggacacuaucuaagacauucaugagucagcucgcauggaagagggcagucugcuucccauuaauggaugugaauccccauaugaacauggugauuugggcggcaucuguagaaaucacaggcgucgaugcgguguuccaaccggccaucccucgugauuuccgcuacuacccuaauguuguggcuaagaacaucggaaggaucagaaagcuguaaaugugcacccaucagagaccugcgacaaugccccaagcagacaccaccuggcagucggagccaccgggucacuccuugucuuaaauaagaaaaacuuagggauaaagucccuugugagugcuugguuaauuaagcuuucaccucaaacaagcacagaucauggauggugauaggggcaaacgugacucguacuggucuacuucuccuagugguagcaccacaaaaccagcaucagguugggagaggucaaguaaagccgacacaugguugcugauucucucauucacccagugggcuuugucaauugccacagugaucaucuguaucauaauuucugcuagacaaggguauaguaugaaagaguacucaaugacuguagaggcauugaacaugagcagcagggaggugaaagagucacuuaccagucuaauaaggcaagagguuauagcaagggcugucaacauucagagcucugugcaaaccggaaucccagucuuguugaacaaaaacagcagggaugucauccagaugauugauaagucgugcagcagacaagagcucacucagcacugugagaguacgaucgcaguccaccaugccgauggaauugccccacuugagccacauaguuucuggagaugcccugucggagaaccguaucuuagcucagauccugaaaucucauugcugccugguccgagcuuguuaucugguucuacaacgaucucuggauguguuaggcucccuucacucucaauuggcgaggcaaucuaugccuauucaucaaaucucauuacacaagguugugcugacauagggaaaucauaucagguccugcagcuaggguacauaucacucaauucagauauguucccugaucuuaaccccguagugucccacacuuaugacaucaacgacaaucggaaaucaugcucuguggugacaacccggacuagggguuaucagcuuugcuccaugccgacuguagacgaaagaaccgacuacucuagugaugguauugaggaucugguccuugauguccuggaucucaaagggagaacuaagucucaccgguaucgcaacagcgagguagaucuugaucacccguucucugcacuauaccccaguguaggcaacggcauugcaacagaaggcucauugauauuucuuggguaugguggacuaaccaccccucugcagggugauacaaaauguaggacccaaggaugccaacaggugucgcaagacacaugcaaugaggcucugaaaauuacauggcuaggagggaaacagguggucagcgugaucauccaggucaaugacuaucucucagagaggccaaagauaagagucacaaccauuccaaucacucaaaacuaucucggggcggaagguagauuauuaaaauugggugaucggguguacaucuauacaagaucaucaggcuggcacucucaacugcagauaggaguacuugaugucagccacccuuugacuaucaacuggacaccucaugaagccuugucuagaccaggaaaugaagagugcaauugguacaauaaguguccgaaggaaugcauaucaggcguauacacugaugcuuauccauuguccccugaugcagcuaacgucgcuaccgucacgcuauaugccaauacaucgcgugucaacccaacaaucauguauucuaacacuacuaacauuauaaauauguuaaggauaaaggauguucaauuagaggcugcauauaccacgacaucguguaucacgcauuuugguaaaggcuacugcuuucacaucaucgagaucaaucagaagagccugaauaccuuacagccgaugcucuuuaagacuagcaucccuaaauuaugcaaggccgagucuuaaauuuaacugacuagcaggcuugucggccuugcugacacuagagucaucuccgaacauccacaauaucucucagucucuuacgucucucacaguauuaagaaaaacccagggugaaugggaagcuugccauaggucauggaugggcaggaguccucccaaaacccuucugacauacucuauccagaaugccaccugaacucucccauagucagggggaagauagcacaguugcacgucuuguuagaugugaaccagcccuacagacugaaggacgacagcauaauaaauauuacaaagcacaaaauuaggaacggaggauuguccccccgucaaauuaagaucaggucucuggguaaggcucuucaacgcacaauaaaggauuuagaccgauacacguuugaaccguacccaaccuacucucaggaauuacuuaggcuugauauaccagagauaugugacaaaauccgauccgucuucgcggucucggaucggcugaccagggaguuaucuaguggguuccaggaucuuugguugaauaucuucaagcaacuaggcaauauagaaggaagagagggguacgauccguugcaggauaucggcaccaucccggagauaacugauaaguacagcaggaauagaugguauaggccauuccuaacuugguucagcaucaaauaugacaugcgguggaugcagaagaccagaccggggggaccccucgauaccucuaauucacauaaccuccuagaaugcaaaucauacacucuaguaacauacggagaucuugucaugauacugaacaaguugacauugacaggguauauccuaaccccugagcuggucuugauguauugugauguuguagaaggaagguggaauaugucugcugcagggcaucuagauaagaaguccauugggauaacaagcaaaggugaggaauuaugggaacuaguggauucccucuucucaagucuuggagaggaaauauacaaugucaucgcacuauuggagccccuaucacuugcucucauacaacuaaaugauccuguuauaccucuacguggggcauuuaugaggcauguguugacagagcuacagacuguuuuaacaaguagagacguguacacagaugcugaagcagacacuauuguggagucguuacucgccauuuuccauggaaccucuauugaugagaaagcagagaucuuuuccuucuuuaggacauuuggccaccccagcuuagaggcugucacugccgccgacaagguaagggcccauauguaugcacaaaaggcaauaaagcuuaagacccuauacgagugucaugcaguuuuuugcacuaucaucauaaauggguauagagagaggcauggcggacaguggccccccugugacuucccugaucacgugugucuagaacuaaggaacgcucaaggguccaauacggcaaucucuuaugaaugugcuguagacaacuauacaaguuucauaggcuucaaguuucggaaguuuauagaaccacaacuagaugaagaucucacaauauauaugaaagacaaagcacuaucccccaggaaggaggcaugggacucuguauacccggauaguaaucuguacuauaaagccccagagucugaagagacccggcggcuuauugaaguguucauaaaugaugagaauuucaacccagaagaaauuaucaauuauguggagucaggagauugguugaaagacgaggaguucaacaucucguacagucucaaagagaaagagaucaagcaagagggucgucuauucgcaaaaaugacuuauaagaugcgagccguacaggugcuggcagagacacuacuggcuaaaggaauaggagagcuauucagcgaaaaugggaugguuaaaggagagauagaccuacuuaaaagauugacuacucuuucugucucaggcguccccaggacugauucaguguacaauaacucuaaaucaucagagaagagaaacgaaggcauggaaaauaagaacucugggggguacugggacgaaaagaagagguccagacaugaauucaaggcaacagauucaucaacagacggcuaugaaacguuaaguugcuuccucacaacagaccucaagaaauacugcuuaaacuggagauuugagaguacugcauuguuuggucagagaugcaacgagauauuuggcuucaagaccuucuuuaacuggaugcauccaguccuugaaagguguacaauauauguuggagauccuuacuguccagucgccgaccggaugcaucgacaacuccaggaucaugcagacucuggcauuuucauacauaauccuagggggggcauagaagguuacugccagaagcuguggaccuuaaucucaaucagugcaauccaccuagcagcugugagagugggugucagggucucugcaaugguucagggugacaaucaagcuauagccgugacaucaagaguaccuguagcucagacuuacaagcagaagaaaaaucaugucuaugaggagaucaccaaauauuucggugcucuaagacacgucauguuugauguagggcacgagcuaaaauugaacgagaccaucauuaguagcaagauguuugucuauaguaaaaggauauacuaugaugggaagauuuuaccacagugccugaaagccuugaccaaguguguauucugguccgagacacugguagaugaaaacagaucugcuuguucgaacaucucaacauccauagcaaaagcuaucgaaaauggguauucuccuauacuaggcuacugcauugcguuguauaagaccugucagcaggugugcauaucacuagggaugacuauaaauccaacuaucagcccgaccguaagagaucaauacuuuaaggguaagaauuggcugagaugugcaguguugauuccagcaaauguuggaggauucaacuacaugucuacaucuagaugcuuuguuagaaauauuggagaccccgcaguagcagcccuagcugaucucaaaagauucaucagagcggaucuguuagacaagcagguauuauacagggucaugaaucaagaacccggugacucuaguuuucuagauugggcuucagacccuuauucguguaaccucccgcauucucagaguauaacuacgauuauaaagaauaucacugcuagaucugugcugcaggaauccccgaauccucuacugucuggucucuucaccgagacuaguggagaagaggaucucaaccuggccucguuccuuauggaccggaaagucauccugccgagaguggcucaugagauccuggguaauuccuuaacuggaguuagggaggcgauugcagggaugcuugauacgaccaagucucuagugagagccagcguuaggaaaggaggauuaucauaugggauauugaggaggcuugucaauuaugaucuauugcaguacgagacacugacuagaacucucaggaaaccggugaaagacaacaucgaauaugaguauauguguucaguugagcuagcugucggucuaaggcagaaaauguggauccaccugacuuacgggagacccauacaugggcuagaaacaccagacccuuuagagcucuugaggggaauauuuaucgaagguucagaggugugcaagcuuugcaggucugaaggagcagaccccaucuauacaugguucuaucuuccugacucuauagaccuggacacgcuuacaaacggauguccggcuauaagaauccccuauuuuggaucagccacugaugaaaggucggaagcccaacucggguauguaagaaaucuaagcaaacccgcaaaggcggccauccggauagcuaugguguauacgugggccuacgggacugaugagauaucguggauggaagccgcucuuauagcccaaacaagagcuaaucugagcuuagagaaucuaaagcugcugacuccuguuucaaccuccacuaaucuaucucauagguugaaagauacggcaacccagaugaaguucucuagugcaacacuaguccgugcaagucgguucauaacaauaucaaaugauaacauggcacucaaagaagcaggggagucgaaggauacuaaucucguguaucagcagauuaugcuaacugggcuaagcuuguucgaguucaauaugagauauaagaaagguuccuuagggaagccacugauauugcacuuacaucuuaauaacgggugcuguauaauggaguccccacaggaggcgaauauccccccaagguccacauuagauuuagagauuacacaagagaacaauaaauugaucuaugauccugauccacucaaggauguggaccuugagcuauuuagcaaggucagagauguuguacacacaguugacaugacuuauuggucagaugaugaaguuaucagagcaaccaguaucuguacugcaaugacgauagcugauacaaugucucaauuagauagagacaacuuaaaagagaugaucgcacuaguaaaugacgaugaugucaacagcuugauuacugaguuuauggugauugauguuccuuuauuuugcucaacguucggggguauucuagucaaucaguuugcauacucacucuacggcuuaaacaucagaggaagggaagaaauauggggacauguaguccggauucuuaaagauaccucccacgcaguuuuaaaagucuuaucuaaugcucuaucucaucccaaaaucuucaaacgauucuggaaugcaggugucguggaaccuguguaugggccuaaccucucaaaucaggauaagauacucuuggcccucucugucugugaauauucuguggaucuauucaugcacgauuggcaaggggguguaccgcuugagaucuuuaucugugacaaugacccagauguggccgacaugaggagguccucuuucuuggcaagacaucuugcauaccuaugcagcuuggcagagauaucuagggaugggccaagauuagaaucaaugaacucucuagagaggcucgagucacuaaagaguuaccuggaacucacauuucuugaugacccgguacugagguacagucaguugacuggccuagucaucaaaguauucccaucuacuuugaccuauauccggaagucaucuauaaaaguguuaaggacaagagguauaggagucccugaagucuuagaagauugggaucccgaggcagauaaugcacuguuagaugguaucgcggcagaaauacaacagaauauuccuuugggacaucagacuagagccccuuuuuggggguugagaguauccaagucacagguacugcgucuccggggguacaaggagaucacaagaggugagauaggcagaucagguguuggucugacguuaccauucgauggaagauaucuaucucaccagcugaggcucuuuggcaucaacaguacuagcugcuugaaagcacuugaacuuaccuaccuauugagccccuuaguugacgaagauaaagauaggcuauauuuaggggaaggagcuggggccaugcuuuccuguuaugacgcuacucuuggcccaugcaucaacuauuauaacucagggguauacucuugugaugucaaugggcagagagaguuaaauauauauccugcugagguggcacuagugggaaagaaauuaaacaauguuacuagucugggucaaagaguuaaaguguuauucaacgggaauccuggcucgacauggauugggaaugaugagugugaggcuuugauuuggaaugaauuacagaauagcucgauaggccuaguccacugugacauggagggaggagaucauaaggaugaucaaguuguacugcaugagcauuacaguguaauccggaucgcguaucuggugggggaucgagacguugugcuuauaagcaagauugcucccaggcugggcacggauuggaccaggcagcucagccuauaucugagauacugggacgagguuaaccuaauagugcuuaaaacaucuaacccugcuuccacagagauguaucuccuaucgaggcaccccaaaucugacauuauagaggacagcaagacaguguuagcuagucuccucccuuugucaaaagaagauagcaucaagauagaaaaguggaucuuaauagagaaggcaaaggcucacgaauggguuacucgggaauugagagaaggaagcucuucaucagggaugcuuagaccuuaccaucaagcacugcagacguuuggcuuugaaccaaacuuguauaaauugagcagagauuucuuguccaccaugaacauagcugauacacacaacugcaugauagcuuucaacaggguuuugaaggauacaaucuucgaaugggcuagaauaacugagucagauaaaaggcuuaaacuaacugguaaguaugaccuguauccugugagagauucaggcaaguugaagacaauuucuagaagacuugugcuaucuuggauaucuuuaucuauguccacaagauugguaacugggucauucccugaccagaaguuugaagcaagacuucaauugggaauaguuucauuaucaucccgugaaaucaggaaccugaggguuaucacaaaaacuuuauuagacagguuugaggauauuauacauaguauaacguauagauuccucaccaaagaaauaaagauuuugaugaagauuuuaggggcagucaagauguucggggccaggcaaaaugaauacacgaccgugauugaugauggaucacuaggugauaucgagccauaugacagcucguaauaauuagucccuaucgugcagaacgaucgaagcuccgcgguaccuggaagucuuggacuuguccauaugacaauaguaagaaaaacuuacaagaagacaagaaaauuuaaaaggauacauaucucuuaaacucuugucuggu